(19) United States
(12) Patent Application Publication
(10) Pub. No.: US 2020/0157145 A1
(43) Pub. Date: May 21, 2020

(54) NEUTRAL-CATIONIC PEPTOIDS AND USES THEREOF

(71) Applicant: Stealth BioTherapeutics Corp, Monaco (MC)

(72) Inventor: Scott DUNCAN, Bedford, MA (US)

(73) Assignee: Stealth BioTherapeutics Corp, Monaco (MC)

(21) Appl. No.: 16/685,604

(22) Filed: Nov. 15, 2019

Related U.S. Application Data
(60) Provisional application No. 62/769,333, filed on Nov. 19, 2018.

(51) Int. Cl. C07K 5/107; A61K 47/12

(52) U.S. Cl. CPC C07K 5/1016; A61K 38/00; A61K 47/12

(57) ABSTRACT

Disclosed herein are methods and compositions for the treatment and/or prevention of diseases or conditions comprising administration of a neutral-cationic peptoid, and/or tautomers, regioisomers, stereoisomers, derivatives, analogues, or pharmaceutically acceptable salts thereof. In some embodiments, the neutral-cationic peptoid comprises η2′,6′-Dmt-ηArg-ηPhe-ηLys-NH₂, ηPhe-ηArg-ηPhe-ηLys-NH₂, or ηArg-η2′,6′-Dmt-ηLys-ηPhe-NH₂.

### CROSS-REFERENCE TO RELATED APPLICATION

This application claims the benefit of priority from U.S. Provisional Patent Application No. 62/769,333, filed Nov. 19, 2018. The contents of this application is incorporated herein by reference in its entirety.

### TECHNICAL FIELD

Disclosed herein are methods and compositions related to the treatment and/or amelioration of diseases and conditions comprising administration of a neutral-cationic peptoid and/or tautomers, regioisomers, stereoisomers, derivatives, analogues, or pharmaceutically acceptable salts thereof. The present technology relates generally to neutral-cationic peptoid compositions and their use in the prevention and treatment of medical diseases and conditions.

### BACKGROUND

The following description is provided to assist the understanding of the reader. None of the information provided or references cited is admitted to be prior art.

Biological cells are generally highly selective as to the molecules that are allowed to pass through the cell membrane. As such, the delivery of compounds, such as small molecules and biological molecules into a cell is usually limited by the physical properties of the compound. The small molecules and biological molecules may, for example, be pharmaceutically active compounds.

### SUMMARY

The present technology provides compositions and methods useful in the prevention, treatment and/or amelioration of diseases and conditions.

In one aspect, the present disclosure provides a composition comprising a neutral-cationic peptoid, tautomers, regioisomers, stereoisomers, derivatives, analogues, or pharmaceutically acceptable salts thereof. In some embodiments, the neutral-cationic peptoid includes any one or more of the neutral-cationic peptoids shown in Section II. In some embodiments, the neutral-cationic peptoid is 2′,6′-dimethyl-ηTyr (“η2′,6′-Dmt”)-ηArg-ηPhe-ηLys-NH₂, ηPhe-ηArg-ηPhe-ηLys-NH₂, or ηArg-η2′,6′-Dmt-ηLys-ηPhe-NH₂.

In another aspect, the present technology provides methods for treating, ameliorating or preventing a medical disease or condition in a subject in need thereof, comprising administering a therapeutically effective amount of a composition comprising a neutral-cationic peptoid of the present technology to the subject thereby treating, amelioration or preventing the medical disease or condition.

In some embodiments, the medical disease or condition comprises ischemia, reperfusion, ischemic heart disease, vessel occlusion injury, and/or myocardial infarction.

In some embodiments, the subject is suffering from ischemia or has an anatomic zone of no-reflow in one or more of cardiovascular tissue, skeletal muscle tissue, cerebral tissue and renal tissue.

In another aspect, the present technology provides methods for treating or preventing no reflow following ischemia-reperfusion injury in a subject in need thereof, comprising administering to the subject a therapeutically effective amount of a composition comprising a neutral-cationic peptoid of the present technology.

In some embodiments, the peptoid is defined by Formula I:

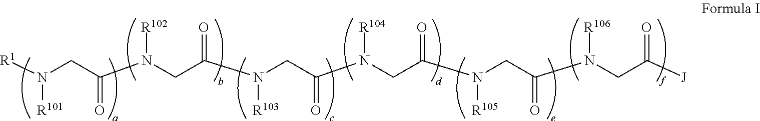

wherein:
J is —N(R³)(R⁴) or —O—R⁵;
R¹⁰¹ is

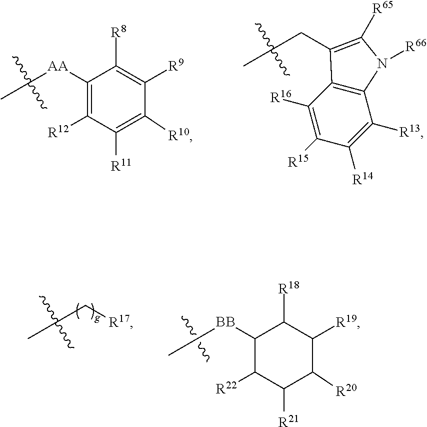

or R²;
R¹⁰² is

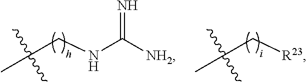

or hydrogen, or optionally R² if a is 0;
R¹⁰³ is

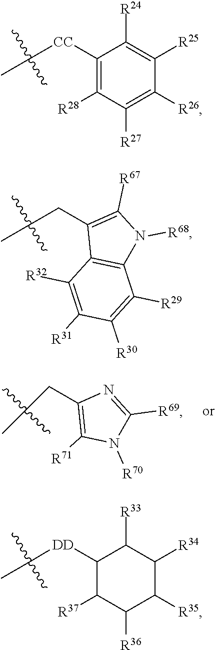

or optionally R² if a and b are each 0;
R¹⁰⁴ is

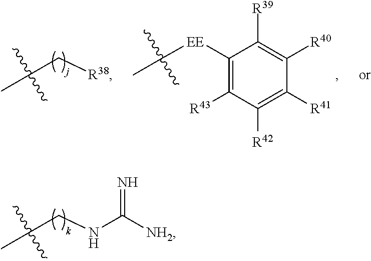

or optionally R² if a, b, and c are each 0;
R¹⁰⁵ is

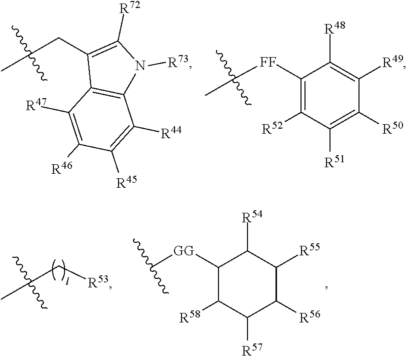

or optionally R² if a, b, c, and d are each 0;
R¹⁰⁶ is

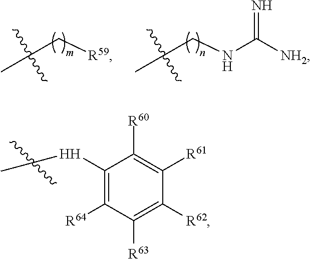

or hydrogen;
   wherein
      R¹, R², R³, R⁴, and R⁵ are each independently a hydrogen or substituted or unsubstituted C₁-C₆ alkyl, C₂-C₆ alkenyl, C₂-C₆ alkynyl, saturated or unsaturated cycloalkyl, cycloalkylalkyl, aryl, aralkyl, 5- or 6-membered saturated or unsaturated heterocylyl, heteroaryl, or amino protecting group; or R¹ and R² together or R³ and R⁴ together form a 3, 4, 5, 6, 7, or 8 membered substituted or unsubstituted heterocycyl ring;
      R⁸, R⁹, R¹⁰, R¹¹, R¹², R¹³, R¹⁴, R¹⁵, R¹⁶, R¹⁸, R¹⁹, R²⁰, R²¹, R²², R²⁴, R²⁵, R²⁶, R²⁷, R²⁸, R²⁹, R³⁰, R³¹, R³², R³³, R³⁴, R³⁵, R³⁶, R³⁷, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵⁴, R⁵⁵, R⁵⁶, R⁵⁷, R⁵⁸, R⁶⁰, R⁶¹, R⁶², R⁶³, R⁶⁴, R⁶⁵, R⁶⁷, R⁶⁹, R⁷¹, and R⁷² are each independently a hydrogen, amino, amido, —NO₂, —CN, —OR^{a}, —SR^{a}, —NR^{a}R^{a}, —F, —Cl, —Br, —I, or a substituted or unsubstituted C₁-C₆ alkyl, C₁-C₆ alkoxy, —C(O)-alkyl, —C(O)-aryl, —C(O)-aralkyl, —C(O)₂R^{a}, C₁-C₄ alkylamino, C₁-C₄ dialkylamino, or perhaloalkyl group;
      R⁶⁶, R⁶⁸, R⁷⁰, and R⁷³ are each independently a hydrogen or substituted or unsubstituted C₁-C₆ alkyl group;
      R¹⁷, R²³, R³⁸, R⁵³, and R⁵⁹ are each independently a hydrogen, —OR^{a}, —SR^{a}, —NR^{a}R^{a}, —NR^{a}R^{b}, —CO₂R^{a}, —(CO)NR^{a}R^{a}, —NR^{a}(CO)R^{a}, —NR^{a}C(NH)NH₂, —NR^{a}-dansyl, enamine, imine, or a substituted or unsubstituted alkyl, heterocyclyl, aryl, heteroaryl, or aralkyl group;
      AA, BB, CC, DD, EE, FF, GG, and HH are each independently absent, —NH(CO)—, or —CH₂—;
      R^{a} at each occurrence is independently a hydrogen or a substituted or unsubstituted C₁-C₆ alkyl group;
      R^{b} at each occurrence is independently a C₁-C₆ alkylene-NR^{a}-dansyl or C₁-C₆ alkylene-NR^{a}-anthraniloyl group;
      a, b, c, d, e, and f are each independently 0 or 1,
         with the proviso that a+b+c+d+e+f≥2; and
      g, h, i, j, k, l, m, and n are independently at each occurrence 1, 2, 3, 4, or 5.

In any embodiment herein of a peptoid of Formula I, it may be that
R¹, R², R³, R⁴, and R⁵ are each independently a hydrogen or substituted or unsubstituted C₁-C₆ alkyl group;
R⁸, R¹², R¹⁸, R²², R²⁴, R²⁸, R³³, R³⁷, R³⁹, R⁴³, R⁴⁸, R⁵², R⁵⁴, R⁵⁸, R⁶⁰, and R⁶⁴ are each independently a hydrogen or methyl group;
R¹⁰, R²⁰, R²⁶, R³⁵, R⁴¹, R⁵⁰, R⁵⁶, and R⁶² are each independently a hydrogen or —OR^{a};
R⁹, R¹¹, R¹⁹, R²¹, R²⁵, R²⁷, R³⁴, R³⁶, R⁴⁰, R⁴², R⁴⁹, R⁵¹, R⁵⁵, R⁵⁷, R⁶¹, R⁶³, R⁶⁵, R⁶⁶, R⁶⁷, R⁶⁸, R⁶⁹, R⁷⁰, R⁷¹, R⁷², and R⁷³ are each a hydrogen;
R¹⁷, R²³, R³⁸, R⁵³, and R⁵⁹ are each independently a hydrogen, —OH, —SH, —SCH₃, —NH₂, —NHR^{b}, —CO₂H, —(CO)NH₂, —NH(CO)H, or —NH-dansyl group;
AA, BB, CC, DD, EE, FF, GG, and HH are each independently absent or —CH₂—;
R^{b} at each occurrence is independently an ethylene-NH-dansyl or ethylene-NH-anthraniloyl group.

In some embodiments of Formula I, at least one of R¹⁰¹, R¹⁰², R¹⁰⁴, R¹⁰⁵, and R¹⁰⁶ is a basic group, as defined above, and at least one of R¹⁰¹, R¹⁰³, R¹⁰⁴, R¹⁰⁵, and R¹⁰⁶ is a neutral group as defined above. In some such embodiments, the neutral group is an aromatic, heterocyclic or cycloalkyl group as defined above. In some embodiments of Formula I, the peptoid contains at least one cationic residue such as ηarginine, and at least one neutral residue such as η2′,6′-dimethyltyrosine, ηtyrosine, or ηphenylalanine. In some embodiments of Formula I, R¹⁰¹ is an alkylguanidinium group.

In some embodiments, the peptoid is defined by Formula II:

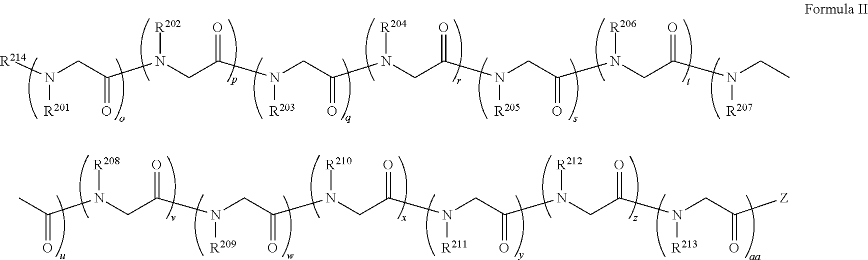

wherein in Formula II:
Z is —N(R²¹⁶)(R²¹⁷) or —O—R²¹⁸;
R²⁰¹ is

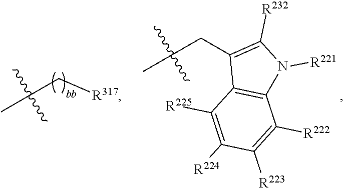

or R²¹⁵;
R²⁰² is

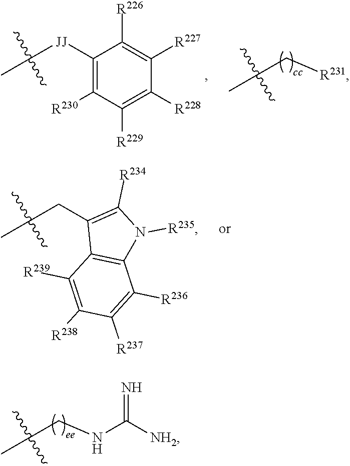

or optionally R²¹⁵ if o is 0;
R²⁰³ is

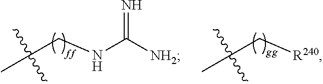

or hydrogen, or optionally R²¹⁵ if o and p are each 0;
R²⁰⁴ is

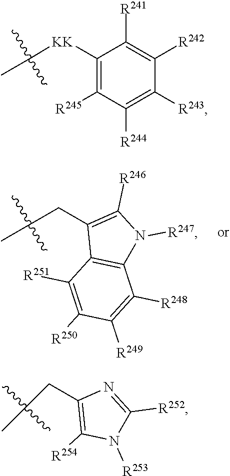

or optionally R²¹⁵ if o, p, and q are each 0;
R²⁰⁵ is

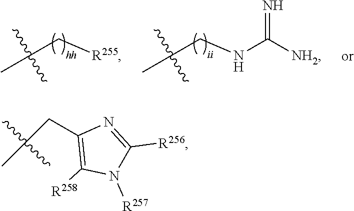

or optionally R²¹⁵ if o, p, q, and r are each 0;
R²⁰⁶ is

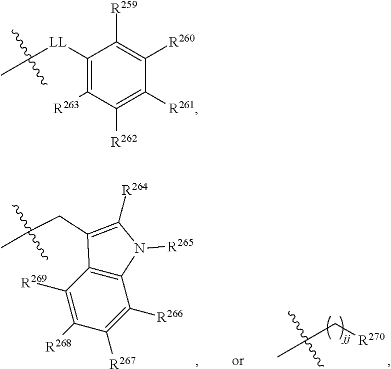

or optionally R²¹⁵ if o, p, q, r, and s are each 0;
R²⁰⁷ is

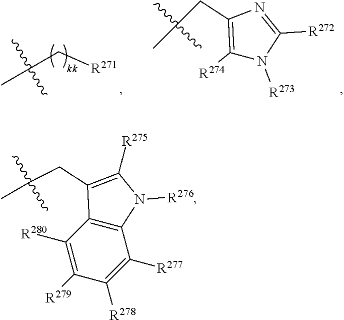

or hydrogen, or optionally R²¹⁵ if o, p, q, r, s, and t are each 0;
R²⁰⁸ is

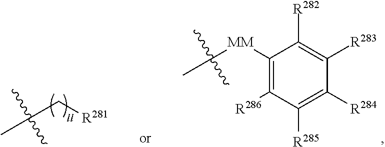

or optionally R²¹⁵ if o, p, q, r, s, t, and u are each 0;
R²⁰⁹ is

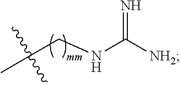

R²¹⁰ is

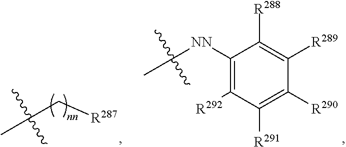

or hydrogen;
R²¹¹ is

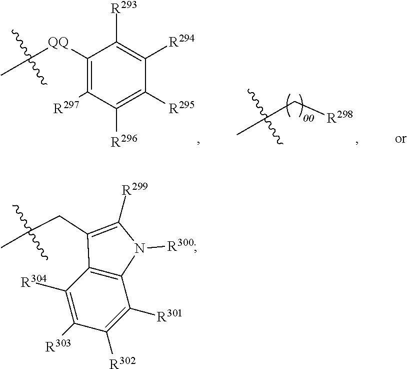

R²¹² is

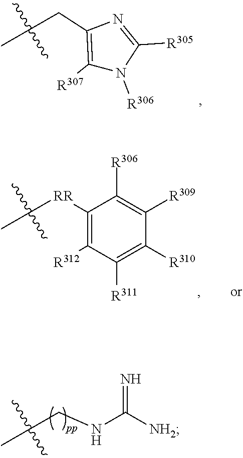

R²¹³ is

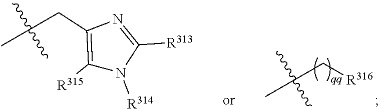

wherein
   R²¹⁴, R²¹⁵, R²¹⁶, R²¹⁷, and R²¹⁸ are each independently a hydrogen or substituted or unsubstituted C₁-C₆ alkyl, C₂-C₆ alkenyl, C₂-C₆ alkynyl, saturated or unsaturated cycloalkyl, cycloalkylalkyl, aryl, aralkyl, 5- or 6-membered saturated or unsaturated heterocylyl, heteroaryl, or amino protecting group; or R²¹⁴ and R²¹⁵ together or R²¹⁶ and R²¹⁷ together form a 3, 4, 5, 6, 7, or 8 membered substituted or unsubstituted heterocycyl ring;
   R²²², R²²³, R²²⁴, R²²⁵, R²²⁶, R²²⁷, R²²⁸, R²²⁹, R²³⁰, R²³², R²³⁴, R²³⁶, R²³⁷, R²³⁸, R²³⁹, R²⁴¹, R²⁴², R²⁴³, R²⁴⁴, R²⁴⁵, R²⁴⁶, R²⁴⁸, R²⁴⁹, R²⁵⁰, R²⁵¹, R²⁵², R²⁵⁴, R²⁵⁶, R²⁵⁸, R²⁵⁹, R²⁶⁰, R²⁶¹, R²⁶², R²⁶³, R²⁶⁴, R²⁶⁶, R²⁶⁷, R²⁶⁸, R²⁶⁹, R²⁷², R²⁷⁴, R²⁷⁵, R²⁷⁷, R²⁷⁸, R²⁷⁹, R²⁸⁰, R²⁸², R²⁸³, R²⁸⁴, R²⁸⁵, R²⁸⁶, R²⁸⁸, R²⁸⁹, R²⁹⁰, R²⁹¹, R²⁹², R²⁹³, R²⁹⁴, R²⁹⁵, R²⁹⁶, R²⁹⁷, R²⁹⁹, R³⁰¹, R³⁰², R³⁰³, R³⁰⁴, R³⁰⁵, R³⁰⁷, R³⁰⁸, R³⁰⁹, R³¹⁰, R³¹¹, R³¹², R³¹³, and R³¹⁵ are each independently a hydrogen, amino, amido, —NO₂, —CN, —OR^{c}, —SR^{c}, —NR^{c}R^{c}, —F, —Cl, —Br, —I, or a substituted or unsubstituted C₁-C₆ alkyl, C₁-C₆ alkoxy, —C(O)-alkyl, —C(O)-aryl, —C(O)-aralkyl, —C(O)₂R^{c}, C₁-C₄ alkylamino, C₁-C₄ dialkylamino, or perhaloalkyl group;
   R²²¹, R²³⁵, R²⁴⁷, R²⁵³, R²⁵⁷, R²⁶⁵, R²⁷³, R²⁷⁶, R³⁰⁰, R³⁰⁶, and R³¹⁴ are each independently a hydrogen or substituted or unsubstituted C₁-C₆ alkyl group;
   R²³¹, R²⁴⁰, R²⁵⁵, R²⁷⁰, R²⁷¹, R²⁸¹, R²⁸⁷, R²⁹⁸, R³¹⁶, and R³¹⁷ are each independently a hydrogen, —OR^{c}, —SR^{c}, —NR^{c}R^{c}, —NR^{c}R^{d}, —CO₂R^{c}, —(CO)NR^{c}R^{c}, —NR^{c}(CO)R^{c}, —NR^{c}C(NH)NH₂, —NR^{c}-dansyl, enamine, imine, or a substituted or unsubstituted alkyl, heterocyclyl, aryl, heteroaryl, or aralkyl group;
   JJ, KK, LL, MM, NN, QQ, and RR are each independently absent, —NH(CO)—, or —CH₂—;
   R^{c} at each occurrence is independently a hydrogen or a substituted or unsubstituted C₁-C₆ alkyl group;
   R^{d} at each occurrence is independently a C₁-C₆ alkylene-NR^{c}-dansyl or C₁-C₆ alkylene-NR^{c}-anthraniloyl group;
   o, p, q, r, s, t, u, v, w, x, y, z, and aa are each independently 0 or 1,
      with the proviso that o+p+q+r+s+t+u+v+w+x+y+z+aa equals 6, 7, 8, 9, 10, or 11;
   cc is 0, 1, 2, 3, 4, or 5; and
   bb, cc, ee, ff gg, hh, ii, jj, kk, ii, mm, nn, oo, pp, and qq are each independently 1, 2, 3, 4, or 5.

In some embodiments of the present technology, the peptoid may be of Formula III:

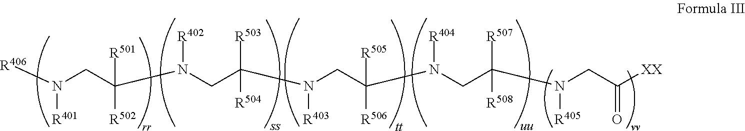

wherein:
XX is —N(R⁴⁰⁸)(R⁴⁰⁹) or —O—R⁴¹⁰;
R⁴⁰¹ is

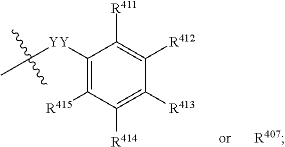

R⁴⁰² is

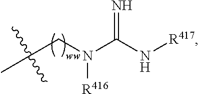

or optionally R⁴⁰⁷ if rr is 0;
R⁴⁰³ is

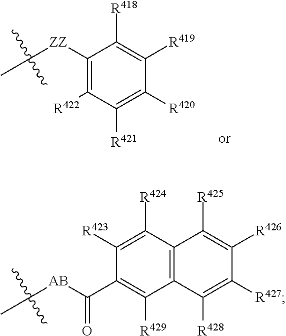

R⁴⁰⁴ is

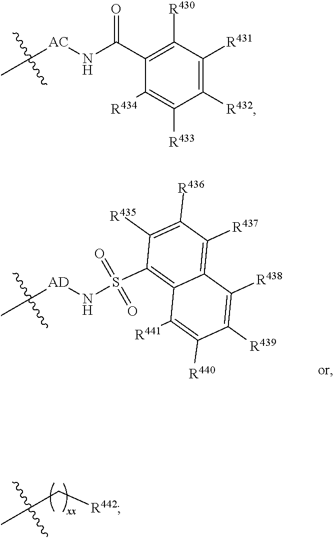

R⁴⁰⁵ is

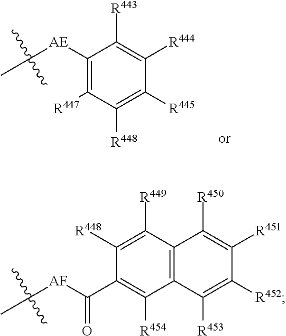

wherein
   R⁴⁰⁶, R⁴⁰⁷, R⁴⁰⁸, R⁴⁰⁹, and R⁴¹⁰ are each independently a hydrogen or substituted or unsubstituted C₁-C₆ alkyl, C₂-C₆ alkenyl, C₂-C₆ alkynyl, saturated or unsaturated cycloalkyl, cycloalkylalkyl, aryl, aralkyl, 5- or 6-membered saturated or unsaturated heterocylyl, heterobicyclyl, heteroaryl, or amino protecting group; or R⁴⁰⁶ and R⁴⁰⁷ together or R⁴⁰⁸ and R⁴⁰⁹ together form a 3-, 4-, 5-, 6-, 7-, or 8-member substituted or unsubstituted heterocycyl ring;
   R⁵⁰¹ and R⁵⁰² are each independently a hydrogen or substituted or unsubstituted C₁-C₆ alkyl group; or together R⁵⁰¹ and R⁵⁰² are C═O;
   R⁵⁰³ and R⁵⁰⁴ are each independently a hydrogen or substituted or unsubstituted C₁-C₆ alkyl group; or together R⁵⁰³ and R⁵⁰⁴ are C═O;
   R⁵⁰⁵ and R⁵⁰⁶ are each independently a hydrogen or substituted or unsubstituted C₁-C₆ alkyl group; or together R⁵⁰⁵ and R⁵⁰⁶ are C═O;
   R⁵⁰⁷ and R⁵⁰⁸ are each independently a hydrogen or substituted or unsubstituted C₁-C₆ alkyl group; or together R⁵⁰⁷ and R⁵⁰⁸ are C═O;
   R⁴¹¹, R⁴¹², R⁴¹³, R⁴¹⁴, R⁴¹⁵, R⁴¹⁸, R⁴¹⁹, R⁴²⁰, R⁴²¹, R⁴²², R⁴²³, R⁴²⁴, R⁴²⁵, R⁴²⁶, R⁴²⁷, R⁴²⁸, R⁴²⁹, R⁴³⁰, R⁴³¹, R⁴³², R⁴³³, R⁴³⁴, R⁴³⁵, R⁴³⁶, R⁴³⁷, R⁴³⁸, R⁴³⁹, R⁴⁴⁰, R⁴⁴¹, R⁴⁴³, R⁴⁴⁴, R⁴⁴⁵, R⁴⁴⁶, R⁴⁴⁷, R⁴⁴⁸, R⁴⁴⁹, R⁴⁵⁰, R⁴⁵¹, R⁴⁵², R⁴⁵³, and R⁴⁵⁴ are each independently a hydrogen, deuterium, amino, amido, —NO₂, —CN, —OR^{e}, —SR^{e}, —NR^{e}R^{e}, —F, —Cl, —Br, —I, or a substituted or unsubstituted C₁-C₆ alkyl, C₁-C₆ alkoxy, —C(O)-alkyl, —C(O)-aryl, —C(O)-aralkyl, —C(O)₂R^{e}, C₁-C₄ alkylamino, C₁-C₄ dialkylamino, or perhaloalkyl group;
   R⁴¹⁶ and R⁴¹⁷ are each independently a hydrogen, —C(O)R^{e}, or a substituted or unsubstituted C₁-C₆ alkyl;
   R⁴⁴² is a hydrogen, —OR^{e}, —SR^{e}, —NR^{e}R^{e}, —NR^{e}R^{f}, —CO₂R^{e}, —C(O)NR^{e}R^{e}, —NR^{e}C(O)R^{e}, —NR^{e}C(NH)NH₂, —NR^{e}-dansyl, enamine, imine, or a substituted or unsubstituted alkyl, heterocyclyl, aryl, heteroaryl, or aralkyl group;
   YY, ZZ, and AE are each independently absent, —NH(CO)—, or —CH₂—;
   AB, AC, AD, and AF are each independently absent or C₁-C₆ alkylene group;
   R^{e} at each occurrence is independently a hydrogen or a substituted or unsubstituted C₁-C₆ alkyl group;
   R^{f} at each occurrence is independently a C₁-C₆ alkylene-NR^{e}-dansyl or C₁-C₆ alkylene-NR^{e}-anthraniloyl group;
   rr, ss, and vv are each independently 0 or 1; tt and uu are each 1
      with the proviso that rr+ss+tt+uu+vv equals 4 or 5; and
   ww and xx are each independently 1, 2, 3, 4, or 5.
   with the proviso that when vv is 0, then uu is 1 and together R⁵⁰⁷ and R⁵⁰⁸ are C═O.

In some embodiments, the peptoid is defined by Formula IV:

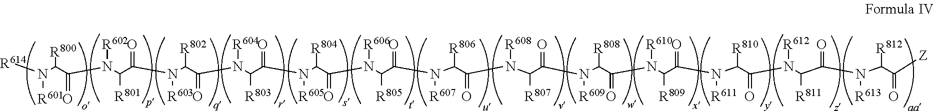

wherein:
Z is —N(R⁶¹⁶)(R⁶¹⁷) or —O—R⁶¹⁸;
R⁶⁰¹ is

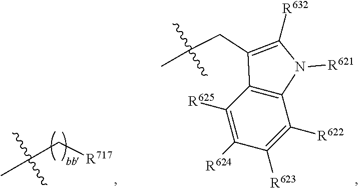

or together with R⁸⁰⁰ is a substituted or unsubstituted C₃ alkyenyl group, or is R⁶¹⁵, provided that when R⁸⁰⁰ is not hydrogen or together with R⁶⁰¹ a substituted or unsubstituted C₃ alkyenyl group then R⁶⁰¹ is hydrogen;
R⁶⁰² is

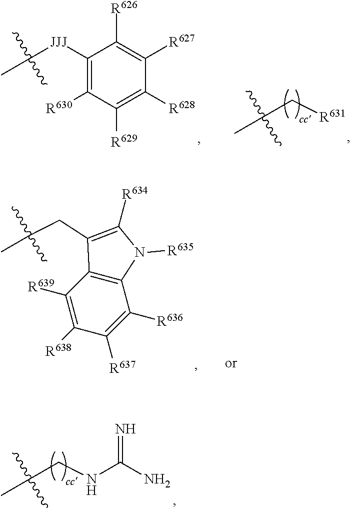

or together with R⁸⁰¹ is a substituted or unsubstituted C₃ alkyenyl group, or optionally R⁶¹⁵ if o′ is 0, provided that when R⁸⁰¹ is not hydrogen or together with R⁶⁰² a substituted or unsubstituted C₃ alkyenyl group then R⁶⁰² is hydrogen;
R⁶⁰³ is

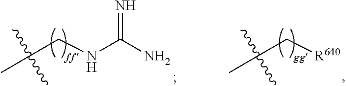

or hydrogen, or together with R⁸⁰² is a substituted or unsubstituted C₃ alkyenyl group, or optionally R⁶¹⁵ if o′ and p′ are each 0, with the proviso that when R⁸⁰² is not hydrogen or together with R⁶⁰³ a substituted or unsubstituted C₃ alkyenyl group then R⁶⁰³ is hydrogen;
R⁶⁰⁴ is

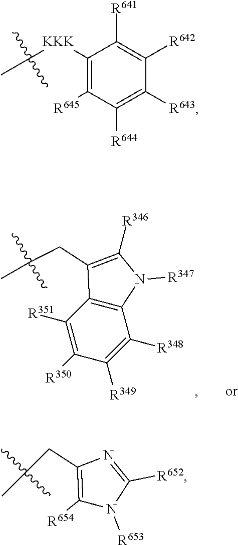

or together with R⁸⁰³ is a substituted or unsubstituted C₃ alkyenyl group, or optionally R⁶¹⁵ if o′, p′, and q′ are each 0, with the proviso that when R⁸⁰³ is not hydrogen or together with R⁶⁰⁴ a substituted or unsubstituted C₃ alkyenyl group then R⁶⁰⁴ is hydrogen;
R⁶⁰⁵ is

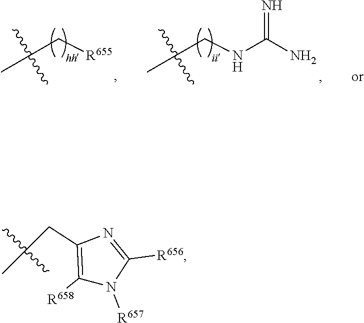

or together with R⁸⁰⁴ is a substituted or unsubstituted C₃ alkyenyl group, or optionally R⁶¹⁵ if o′, p′, q′, and r′ are each 0, with the proviso that when R⁸⁰⁴ is not hydrogen or together with R⁶⁰⁵ a substituted or unsubstituted C₃ alkyenyl group then R⁶⁰⁵ is hydrogen;
R⁶⁰⁶ is

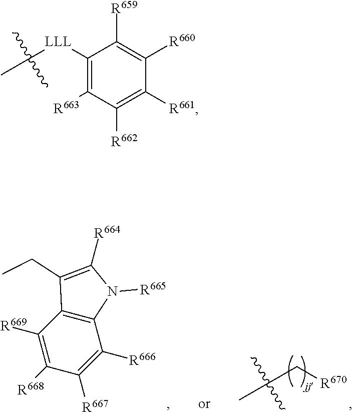

or together with R⁸⁰⁵ is a substituted or unsubstituted C₃ alkyenyl group, or optionally R⁶¹⁵ if o′, p′, q′, r′, and s′ are each 0, with the proviso that when R⁸⁰⁵ is not hydrogen or together with R⁶⁰⁶ a substituted or unsubstituted C₃ alkyenyl group then R⁶⁰⁶ is hydrogen;
R⁶⁰⁷ is

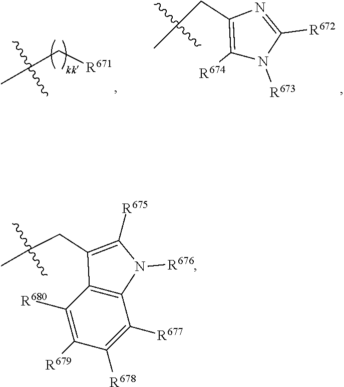

or hydrogen, or together with R⁸⁰⁶ is a substituted or unsubstituted C₃ alkyenyl group, or optionally R⁶¹⁵ if o′, p′, q′, r′, s′, and t′ are each 0, with the proviso that when R⁸⁰⁶ is not hydrogen or together with R⁶⁰⁷ a substituted or unsubstituted C₃ alkyenyl group then R⁶⁰⁷ is hydrogen;
R⁶⁰⁸ is

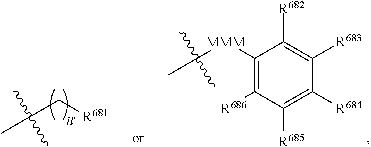

or R⁶⁸⁵ or together with R⁸⁰⁷ is a substituted or unsubstituted C₃ alkyenyl group, or optionally R⁶¹⁵ if o′, p′, q′, r′, s′, t′, and u′ are each 0, with the proviso that when R⁸⁰⁷ is not hydrogen or together with R⁶⁰⁸ a substituted or unsubstituted C₃ alkyenyl group then R⁶⁰⁸ is hydrogen;
R⁶⁰⁹ is

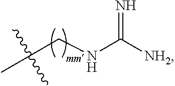

or together with R⁸⁰⁸ is a substituted or unsubstituted C₃ alkyenyl group, or optionally R⁶¹⁵ if o′, p′, q′, r′, s′, t′, u′, and v′ are each 0, with the proviso that when R⁸⁰⁸ is not hydrogen or together with R⁶⁰⁹ a substituted or unsubstituted C₃ alkyenyl group then R⁶⁰⁹ is hydrogen;
R⁶¹⁰ is

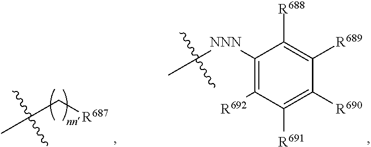

or hydrogen, or together with R⁸⁰⁹ is a substituted or unsubstituted C₃ alkyenyl group, or optionally R⁶¹⁵ if o′, p′, q′, r′, s′, t′, u′, v′, and w′ are each 0, with the proviso that when R⁸⁰⁹ is not hydrogen or together with R⁶¹⁰ a substituted or unsubstituted C₃ alkyenyl group then R⁶¹⁰ is hydrogen;
R⁶¹¹ is

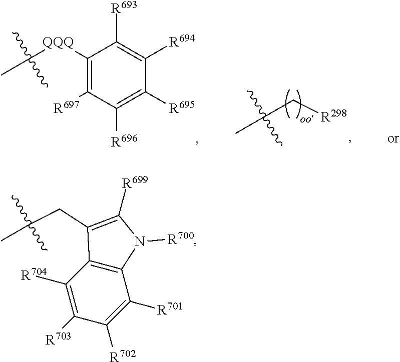

or together with R⁸¹⁰ is a substituted or unsubstituted C₃ alkyenyl group, with the proviso that when R⁸¹⁰ is not hydrogen or together with R⁶¹¹ a substituted or unsubstituted C₃ alkyenyl group then R⁶¹¹ is hydrogen;
R⁶¹² is

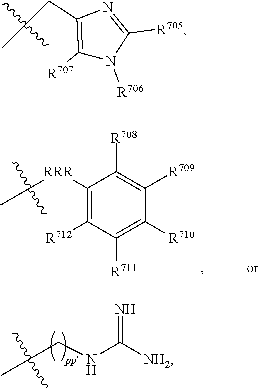

or together with R⁸¹¹ is a substituted or unsubstituted C₃ alkyenyl group, with the proviso that when R⁸¹¹ is not hydrogen or together with R⁶¹² a substituted or unsubstituted C₃ alkyenyl group then R⁶¹² is hydrogen;
R⁶¹³ is

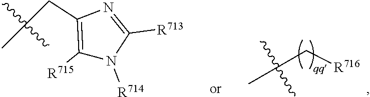

or together with R⁸¹² is a substituted or unsubstituted C₃ alkyenyl group, with the proviso that when R⁸¹² is not hydrogen or together with R⁶¹³ a substituted or unsubstituted C₃ alkyenyl group then R⁶¹³ is hydrogen;
one or two of R⁸⁰⁰, R⁸⁰¹, R⁸⁰², R⁸⁰³, R⁸⁰⁴, R⁸⁰⁵, R⁸⁰⁶, R⁸⁰⁷, R⁸⁰⁸, R⁸⁰⁹, R⁸¹⁰, R⁸¹¹, and R⁸¹² are each independently the aforementioned substituted or unsubstituted C₃ alkyenyl group,

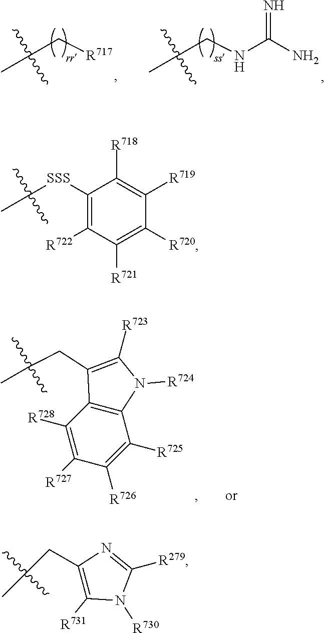

and the remaining R⁸⁰⁰, R⁸⁰¹, R⁸⁰², R⁸⁰³, R⁸⁰⁴, R⁸⁰⁵, R⁸⁰⁶, R⁸⁰⁷, R⁸⁰⁸, R⁸⁰⁹, R⁸¹⁰, R⁸¹¹, and R⁸¹² are each hydrogen,
   wherein
      R⁶¹⁴, R⁶¹⁵, R⁶¹⁶, R⁶¹⁷, and R⁶¹⁸ are each independently a hydrogen or substituted or unsubstituted C₁-C₆ alkyl, C₂-C₆ alkenyl, C₂-C₆ alkynyl, saturated or unsaturated cycloalkyl, cycloalkylalkyl, aryl, aralkyl, 5- or 6-membered saturated or unsaturated heterocylyl, heteroaryl, or amino protecting group; or R⁶¹⁴ and R⁶¹⁵ together or R⁶¹⁶ and R⁶¹⁷ together form a 3, 4, 5, 6, 7, or 8 membered substituted or unsubstituted heterocycyl ring;
      R⁶²², R⁶²³, R⁶²⁴, R⁶²⁵, R⁶²⁶, R⁶²⁷, R⁶²⁸, R⁶²⁹, R⁶³⁰, R⁶³², R⁶³⁴, R⁶³⁶, R⁶³⁷, R⁶³⁸, R⁶³⁹, R⁶⁴¹, R⁶⁴², R⁶⁴³, R⁶⁴⁴, R⁶⁴⁵, R⁶⁴⁶, R⁶⁴⁸, R⁶⁴⁹, R⁶⁵⁰, R⁶⁵¹, R⁶⁵², R⁶⁵⁴, R⁶⁵⁶, R⁶⁵⁸, R⁶⁵⁹, R⁶⁶⁰, R⁶⁶¹, R⁶⁶², R⁶⁶³, R⁶⁶⁴, R⁶⁶⁶, R⁶⁶⁷, R⁶⁶⁸, R⁶⁶⁹, R⁶⁷², R⁶⁷⁴, R⁶⁷⁵, R⁶⁷⁷, R⁶⁷⁸, R⁶⁷⁹, R⁶⁸⁰, R⁶⁸², R⁶⁸³, R⁶⁸⁴, R⁶⁸⁵, R⁶⁸⁶, R⁶⁸⁸, R⁶⁸⁹, R⁶⁹⁰, R⁶⁹¹, R⁶⁹², R⁶⁹³, R⁶⁹⁴, R⁶⁹⁵, R⁶⁹⁶, R⁶⁹⁷, R⁶⁹⁹, R⁷⁰¹, R⁷⁰², R⁷⁰³, R⁷⁰⁴, R⁷⁰⁵, R⁷⁰⁷, R⁷⁰⁸, R⁷⁰⁹, R⁷¹⁰, R⁷¹¹, R⁷¹², R⁷¹³, R⁷¹⁵, R⁷¹⁸, R⁷¹⁹, R⁷²⁰, R⁷²¹, R⁷²², R⁷²³, R⁷²⁵, R⁷²⁶, R⁷²⁷, R⁷²⁸, R⁷³⁰, and R⁷³¹ are each independently a hydrogen, amino, amido, —NO₂, —CN, —OR^{c}, —SR^{c}, —NR^{c}R^{c}, —F, —Cl, —Br, —I, or a substituted or unsubstituted C₁-C₆ alkyl, C₁-C₆ alkoxy, —C(O)-alkyl, —C(O)-aryl, —C(O)-aralkyl, —C(O)₂R^{c}, C₁-C₄ alkylamino, C₁-C₄ dialkylamino, or perhaloalkyl group;
      R⁶²¹, R⁶³⁵, R⁶⁴⁷, R⁶⁵³, R⁶⁵⁷, R⁶⁶⁵, R⁶⁷³, R⁶⁷⁶, R⁷⁰⁰, R⁷⁰⁶, R⁷¹⁴, R⁷²⁴, and R⁷²⁹ are each independently a hydrogen or substituted or unsubstituted C₁-C₆ alkyl group;
      R⁶¹⁶, R⁶¹⁷, R⁶³¹, R⁶⁴⁰, R⁶⁵⁵, R⁶⁷⁰, R⁶⁷¹, R⁶⁸¹, R⁶⁸⁷, R⁶⁹⁸, and R⁷¹⁷ are each independently a hydrogen, —OR^{g}, —SR^{g}, —NR^{g}R^{g}, —NR^{g}R^{h}, —CO₂R^{g}, —(CO)NR^{g}R^{g}, —NR^{g}(CO)R^{g}, —NR^{g}C(NH)NH₂, —NR^{g}-dansyl, enamine, imine, or a substituted or unsubstituted alkyl, heterocyclyl, aryl, heteroaryl, or aralkyl group;
      JJJ, KKK, LLL, MMM, NNN, QQQ, RRR, and SSS are each independently absent, —NH(CO)—, or —CH₂—;
      R^{g} at each occurrence is independently a hydrogen or a substituted or unsubstituted C₁-C₆ alkyl group;
      R^{h} at each occurrence is independently a C₁-C₆ alkylene-NR^{c}-dansyl or C₁-C₆ alkylene-NR^{c}-anthraniloyl group;
      o′, p′, q′, r′, s′, t′, u′, v′, w′, x′, y′, z′, and aa′ are each independently 0 or 1,
         with the proviso that o′+p′+q′+r′+s′+t′+u′+v′+w′+x′+y′+z′+aa′ equals 4, 5, 6, 7, 8, 9, 10, or 11;
      bb′, cc′, ee′, ff′, gg′, hh′, ii′, jj′, kk′, ll′, mm′, nn′, oo′, pp′, qq′, rr′, and ss' are each independently 1, 2, 3, 4, or 5.

In some embodiments, the neutral-cationic peptoids of the present technology have a core structural motif of alternating neutral and cationic peptoid monomers. For example, the peptoid may be a tetrapeptoid defined by any of Formulas A to F set forth below:

Neutral-Cationic-Neutral-Cationic (Formula A)

Cationic-Neutral-Cationic-Neutral (Formula B)

Neutral-Neutral-Cationic-Cationic (Formula C)

Cationic-Cationic-Neutral-Neutral (Formula D)

Neutral-Cationic-Cationic-Neutral (Formula E)

Cationic-Neutral-Neutral-Cationic (Formula F)

In Formulas A-F, Neutral may be a residue selected from the group consisting of: FηPhe (ηF), 2,6-dimethyl-FηPhe (η2,6-DMF), ηTyr (ηY), 2,6-dimethyl-ηTyr (η2,6-DMT), and ηTrp (ηW). In some embodiments, the ηPhe, η2,6-DMF, ηTyr, η2,6-DMT, and/or ηTrp residue may be substituted with a saturated analog, e.g., ηCyclohexylalanine (ηCha) for ηPhe. In some embodiments, Cationic is a residue selected from the group consisting of: ηArg (ηR), ηLys (ηK), and ηHis (ηH). In some embodiments of Formulas A-F, one, two or three of the residues are corresponding alpha-amino acid residues, e.g., Phe, 2,6-dimethyl-Phe, Tyr, 2,6-dimethyl-Tyr, Trp, Cha; and Arg (including, e.g., D-Arg), Lys, and His.

### DETAILED DESCRIPTION

It is to be appreciated that certain aspects, modes, embodiments, variations and features of the present technology are described below in various levels of detail in order to provide a substantial understanding of the present technology.

While the peptoids described herein can occur and can be used as the neutral (non-salt) peptoid, the description is intended to embrace all salts of the peptoids described herein, as well as methods of using such salts of the peptoids. In one embodiment, the salts of the peptoids comprise pharmaceutically acceptable salts. Pharmaceutically acceptable salts are those salts which can be administered as drugs or pharmaceuticals to humans and/or animals and which, upon administration, retain at least some of the biological activity of the free compound (neutral compound or non-salt compound). The desired salt of a basic peptoid may be prepared by methods known to those of skill in the art by treating the compound with an acid. Examples of inorganic acids include, but are not limited to, hydrochloric acid, hydrobromic acid, sulfuric acid, nitric acid, and phosphoric acid. Examples of organic acids include, but are not limited to, formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, sulfonic acids, and salicylic acid. Salts of basic peptoids with amino acids, such as aspartate salts and glutamate salts, can also be prepared. The desired salt of an acidic peptoid can be prepared by methods known to those of skill in the art by treating the compound with a base. Examples of inorganic salts of acids include, but are not limited to, alkali metal and alkaline earth salts, such as sodium salts, potassium salts, magnesium salts, and calcium salts; ammonium salts; and aluminum salts. Examples of organic salts of acid peptoids include, but are not limited to, procaine, dibenzylamine, N-ethylpiperidine, N,N′-dibenzylethylenediamine, and triethylamine salts. Salts of acidic peptoids with amino acids, such as lysine salts, can also be prepared. The present technology also includes all stereoisomers and geometric isomers of the peptoids, including diastereomers, enantiomers, and cis/trans (E/Z) isomers. The present technology also includes mixtures of stereoisomers and/or geometric isomers in any ratio, including, but not limited to, racemic mixtures.

### I. Select Definitions

The definitions of certain terms as used in this specification are provided below. Unless defined otherwise, all technical and scientific terms used herein generally have the same meaning as commonly understood by one of ordinary skill in the art to which this present technology belongs.

As used in this specification and the appended claims, the singular forms “a”, “an” and “the” include plural referents unless the content clearly dictates otherwise. For example, reference to “a cell” includes a combination of two or more cells, and the like.

As used herein, the term “about” encompasses the range of experimental error that may occur in a measurement and will be clear to the skilled artisan. If there are uses of the term which are not clear to persons of ordinary skill in the art, given the context in which it is used, “about” will mean up to plus or minus 10% of the particular term.

Generally, reference to a certain element such as hydrogen or H is meant to include all isotopes of that element. For example, if an R group is defined to include hydrogen or H, it also includes deuterium and tritium. Compounds comprising radioisotopes such as tritium, C14, P32 and S35 are thus within the scope of the present technology. Procedures for inserting such labels into the compounds of the present technology will be readily apparent to those skilled in the art based on the disclosure herein.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, aryloxy, aralkyloxy, heterocyclyl, heterocyclylalkyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls; pentafluorosulfanyl (i.e., SF₅), sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like.

Substituted ring groups such as substituted cycloalkyl, aryl, heterocyclyl and heteroaryl groups also include rings and ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups as defined below.

Alkyl groups include straight chain and branched chain alkyl groups having from 1 to 12 carbon atoms, and typically from 1 to 10 carbons or, in some embodiments, from 1 to 8, 1 to 6, or 1 to 4 carbon atoms. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, alkoxyalkyl, carboxyalkyl, and the like.

Cycloalkyl groups include mono-, bi- or tricyclic alkyl groups having from 3 to 12 carbon atoms in the ring(s), or, in some embodiments, 3 to 10, 3 to 8, or 3 to 4, 5, or 6 carbon atoms. Exemplary monocyclic cycloalkyl groups include, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Bi- and tricyclic ring systems include both bridged cycloalkyl groups and fused rings, such as, but not limited to, bicyclo[2.1.1]hexane, adamantyl, decalinyl, and the like. Substituted cycloalkyl groups may be substituted one or more times with, non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4- 2,5- or 2,6-disubstituted cyclohexyl groups, which may be substituted with substituents such as those listed above.

Cycloalkylalkyl groups are alkyl groups as defined above in which a hydrogen or carbon bond of an alkyl group is replaced with a bond to a cycloalkyl group as defined above. In some embodiments, cycloalkylalkyl groups have from 4 to 16 carbon atoms, 4 to 12 carbon atoms, and typically 4 to 10 carbon atoms. Substituted cycloalkylalkyl groups may be substituted at the alkyl, the cycloalkyl or both the alkyl and cycloalkyl portions of the group. Representative substituted cycloalkylalkyl groups may be mono-substituted or substituted more than once, such as, but not limited to, mono-, di- or tri-substituted with substituents such as those listed above.

Alkenyl groups include straight and branched chain alkyl groups as defined above, except that at least one double bond exists between two carbon atoms. Alkenyl groups have from 2 to 12 carbon atoms, and typically from 2 to 10 carbons or, in some embodiments, from 2 to 8, 2 to 6, or 2 to 4 carbon atoms. In some embodiments, the alkenyl group has one, two, or three carbon-carbon double bonds. Examples include, but are not limited to vinyl, allyl, —CH═CH(CH₃), —CH═C(CH₃)₂, —C(CH₃)═CH₂, —C(CH₃)═CH(CH₃), —C(CH₂CH₃)═CH₂, among others. Representative substituted alkenyl groups may be mono-substituted or substituted more than once, such as, but not limited to, mono-, di- or tri-substituted with substituents such as those listed above.

Cycloalkenyl groups include cycloalkyl groups as defined above, having at least one double bond between two carbon atoms. In some embodiments the cycloalkenyl group may have one, two or three double bonds but does not include aromatic compounds. Cycloalkenyl groups have from 4 to 14 carbon atoms, or, in some embodiments, 5 to 14 carbon atoms, 5 to 10 carbon atoms, or even 5, 6, 7, or 8 carbon atoms. Examples of cycloalkenyl groups include cyclohexenyl, cyclopentenyl, cyclohexadienyl, cyclobutadienyl, and cyclopentadienyl.

Cycloalkenylalkyl groups are alkyl groups as defined above in which a hydrogen or carbon bond of the alkyl group is replaced with a bond to a cycloalkenyl group as defined above. Substituted cycloalkenylalkyl groups may be substituted at the alkyl, the cycloalkenyl or both the alkyl and cycloalkenyl portions of the group. Representative substituted cycloalkenylalkyl groups may be substituted one or more times with substituents such as those listed above.

Alkynyl groups include straight and branched chain alkyl groups as defined above, except that at least one triple bond exists between two carbon atoms. Alkynyl groups have from 2 to 12 carbon atoms, and typically from 2 to 10 carbons or, in some embodiments, from 2 to 8, 2 to 6, or 2 to 4 carbon atoms. In some embodiments, the alkynyl group has one, two, or three carbon-carbon triple bonds. Examples include, but are not limited to —C≡CH, —C≡CCH₃, —CH₂C≡CCH₃, —C≡CCH₂CH(CH₂CH₃)₂, among others. Representative substituted alkynyl groups may be mono-substituted or substituted more than once, such as, but not limited to, mono-, di- or tri-substituted with substituents such as those listed above.

Aryl groups are cyclic aromatic hydrocarbons that do not contain heteroatoms. Aryl groups herein include monocyclic, bicyclic and tricyclic ring systems. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. Although the phrase “aryl groups” includes groups containing fused rings, such as fused aromatic-aliphatic ring systems (e.g., indanyl, tetrahydronaphthyl, and the like), it does not include aryl groups that have other groups, such as alkyl or halo groups, bonded to one of the ring members. Rather, groups such as tolyl are referred to as substituted aryl groups. Representative substituted aryl groups may be mono-substituted or substituted more than once. For example, monosubstituted aryl groups include, but are not limited to, 2-, 3-, 4-, 5-, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. In some embodiments, aralkyl groups contain 7 to 16 carbon atoms, 7 to 14 carbon atoms, or 7 to 10 carbon atoms. Substituted aralkyl groups may be substituted at the alkyl, the aryl or both the alkyl and aryl portions of the group. Representative aralkyl groups include but are not limited to benzyl and phenethyl groups and fused (cycloalkylaryl)alkyl groups such as 4-indanylethyl. Representative substituted aralkyl groups may be substituted one or more times with substituents such as those listed above.

Heterocyclyl groups include aromatic (also referred to as heteroaryl) and non-aromatic ring compounds containing 3 or more ring members, of which one or more is a heteroatom such as, but not limited to, N, O, and S. In some embodiments, the heterocyclyl group contains 1, 2, 3 or 4 heteroatoms. In some embodiments, heterocyclyl groups include mono-, bi- and tricyclic rings having 3 to 16 ring members, whereas other such groups have 3 to 6, 3 to 10, 3 to 12, or 3 to 14 ring members. Heterocyclyl groups encompass aromatic, partially unsaturated and saturated ring systems, such as, for example, imidazolyl, imidazolinyl and imidazolidinyl groups. The phrase “heterocyclyl group” includes fused ring species including those comprising fused aromatic and non-aromatic groups, such as, for example, benzotriazolyl, 2,3-dihydrobenzo[1,4]dioxinyl, benzo[1,3]dioxolyl, and 2,3-dihydro-1H-benzo[e][1,4]diazepinyl. The phrase also includes bridged polycyclic ring systems containing a heteroatom such as, but not limited to, quinuclidyl. However, the phrase does not include heterocyclyl groups that have other groups, such as alkyl, oxo or halo groups, bonded to one of the ring members. Rather, these are referred to as “substituted heterocyclyl groups”. Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, tetrahydroquinolinyl, 1,2-diazepanyl, 1,3-diazepanyl, and 1,4-diazepanyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups are aromatic ring compounds containing 5 or more ring members, of which, one or more is a heteroatom such as, but not limited to, N, O, and S. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, imidazopyridinyl (azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. Although the phrase “heteroaryl groups” includes fused ring compounds, the phrase does not include heteroaryl groups that have other groups bonded to one of the ring members, such as alkyl groups. Rather, heteroaryl groups with such substitution are referred to as “substituted heteroaryl groups.” Representative substituted heteroaryl groups may be substituted one or more times with various substituents such as those listed above.

Heterocyclylalkyl groups are alkyl groups as defined above in which a hydrogen or carbon bond of an alkyl group is replaced with a bond to a heterocyclyl group as defined above. Substituted heterocyclylalkyl groups may be substituted at the alkyl, the heterocyclyl or both the alkyl and heterocyclyl portions of the group. Representative heterocyclyl alkyl groups include, but are not limited to, morpholin-4-yl-ethyl, furan-2-yl-methyl, imidazol-4-yl-methyl, pyridin-3-yl-methyl, tetrahydrofuran-2-yl-ethyl, and indol-2-yl-propyl. Representative substituted heterocyclylalkyl groups may be substituted one or more times with substituents such as those listed above.

Heteroaralkyl groups are alkyl groups as defined above in which a hydrogen or carbon bond of an alkyl group is replaced with a bond to a heteroaryl group as defined above. Substituted heteroaralkyl groups may be substituted at the alkyl, the heteroaryl or both the alkyl and heteroaryl portions of the group. Representative substituted heteroaralkyl groups may be substituted one or more times with substituents such as those listed above.

Groups described herein having two or more points of attachment (i.e., divalent, trivalent, or polyvalent) within the compound of the present technology are designated by use of the suffix, “ene.” For example, divalent alkyl groups are alkylene groups, divalent aryl groups are arylene groups, divalent heteroaryl groups are divalent heteroarylene groups, and so forth. Substituted groups having a single point of attachment to the compound of the present technology are not referred to using the “ene” designation. Thus, e.g., chloroethyl is not referred to herein as chloroethylene.

Alkoxy groups are hydroxyl groups (—OH) in which the bond to the hydrogen atom is replaced by a bond to a carbon atom of a substituted or unsubstituted alkyl group as defined above. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Representative substituted alkoxy groups may be substituted one or more times with substituents such as those listed above.

The terms “alkanoyl” and “alkanoyloxy” as used herein can refer, respectively, to —C(O)-alkyl and —O—C(O)-alkyl groups, where in some embodiments the alkanoyl or alkanoyloxy groups each contain 2-5 carbon atoms. Similarly, the terms “aryloyl” and “aryloyloxy” respectively refer to to —C(O)-aryl and —O—C(O)-aryl groups

The terms “aryloxy” and “arylalkoxy” refer to, respectively, a substituted or unsubstituted aryl group bonded to an oxygen atom and a substituted or unsubstituted aralkyl group bonded to the oxygen atom at the alkyl. Examples include but are not limited to phenoxy, naphthyloxy, and benzyloxy. Representative substituted aryloxy and arylalkoxy groups may be substituted one or more times with substituents such as those listed above.

The term “carboxylic acid” as used herein refers to a compound with a —C(O)OH group. The term “carboxylate” as used herein refers to a —C(O)O⁻ group. A “substituted carboxylate” refers to a —C(O)O-G where G is a carboxylate protecting group. Carboxylate protecting groups are well known to one of ordinary skill in the art. An extensive list of protecting groups for the carboxylate group functionality may be found in Protective Groups in Organic Synthesis, Greene, T. W.; Wuts, P. G. M., John Wiley & Sons, New York, N.Y., (3rd Edition, 1999) which can be added or removed using the procedures set forth therein and which is hereby incorporated by reference in its entirety and for any and all purposes as if fully set forth herein.

The term “ester” as used herein refers to —COOR⁷⁰ groups. R⁷⁰ is a substituted or unsubstituted alkyl, cycloalkyl, alkenyl, alkynyl, aryl, aralkyl, heterocyclylalkyl or heterocyclyl group as defined herein.

The term “amide” (or “amido”) includes C- and N-amide groups, i.e., —C(O)NR⁷¹R⁷², and —NR⁷¹C(O)R⁷² groups, respectively. R⁷¹ and R⁷² are independently hydrogen, or a substituted or unsubstituted alkyl, alkenyl, alkynyl, cycloalkyl, aryl, aralkyl, heterocyclylalkyl or heterocyclyl group as defined herein. Amido groups therefore include but are not limited to carbamoyl groups (—C(O)NH₂) and formamide groups (—NHC(O)H). In some embodiments, the amide is —NR⁷¹C(O)—(C₁₋₅ alkyl) and the group is termed “carbonylamino,” and in others the amide is —NHC(O)-alkyl and the group is termed “alkanoylamino.”

The term “nitrile” or “cyano” as used herein refers to the —CN group.

Urethane groups include N- and O-urethane groups, i.e., —NR⁷³C(O)OR⁷⁴ and —OC(O)NR⁷³R⁷⁴ groups, respectively. R⁷³ and R⁷⁴ are independently a substituted or unsubstituted alkyl, alkenyl, alkynyl, cycloalkyl, aryl, aralkyl, heterocyclylalkyl, or heterocyclyl group as defined herein. R⁷³ may also be H.

The term “amine” (or “amino”) as used herein refers to —NR⁷⁵R⁷⁶ groups, wherein R⁷⁵ and R⁷⁶ are independently hydrogen, or a substituted or unsubstituted alkyl, alkenyl, alkynyl, cycloalkyl, aryl, aralkyl, heterocyclylalkyl or heterocyclyl group as defined herein. In some embodiments, the amine may be alkylamino, dialkylamino, arylamino, or alkylarylamino. In other embodiments, the amine may be NH₂, methylamino, dimethylamino, ethylamino, diethylamino, propylamino, isopropylamino, phenylamino, or benzylamino.

The term “sulfonamido” includes S- and N-sulfonamide groups, i.e., —SO₂NR⁷⁸R⁷⁹ and —NR⁷⁸SO₂R⁷⁹ groups, respectively. R⁷⁸ and R⁷⁹ are independently hydrogen, or a substituted or unsubstituted alkyl, alkenyl, alkynyl, cycloalkyl, aryl, aralkyl, heterocyclylalkyl, or heterocyclyl group as defined herein. Sulfonamido groups therefore include but are not limited to sulfamoyl groups (—SO₂NH₂). In some embodiments herein, the sulfonamido is —NHSO₂-alkyl and is referred to as the “alkylsulfonylamino” group.

The term “thiol” refers to —SH groups, while sulfides include —SR⁸⁰ groups, sulfoxides include —S(O)R⁸¹ groups, sulfones include —SO₂R⁸² groups, and sulfonyls include —SO₂OR⁸³. R⁸⁰, R⁸¹, R⁸², and R⁸³ are each independently a substituted or unsubstituted alkyl, cycloalkyl, alkenyl, alkynyl, aryl aralkyl, heterocyclyl or heterocyclylalkyl group as defined herein. In some embodiments the sulfide is an alkylthio group, —S-alkyl.

The term “urea” refers to —NR⁸⁴—C(O)—NR⁸⁵R⁸⁶ groups. R⁸⁴, R⁸⁵, and R⁸⁶ groups are independently hydrogen, or a substituted or unsubstituted alkyl, alkenyl, alkynyl, cycloalkyl, aryl, aralkyl, heterocyclyl, or heterocyclylalkyl group as defined herein.

The term “amidine” refers to —C(NR⁸⁷)NR⁸⁸R⁸⁹ and —NR⁸⁷C(NR⁸⁸)R⁸⁹, wherein R⁸⁷, R⁸⁸, and R⁸⁹ are each independently hydrogen, or a substituted or unsubstituted alkyl, cycloalkyl, alkenyl, alkynyl, aryl aralkyl, heterocyclyl or heterocyclylalkyl group as defined herein.

The term “guanidine” refers to —NR⁹⁰C(NR⁹¹)NR⁹²R⁹³, wherein R⁹⁰, R⁹¹, R⁹² and R⁹³ are each independently hydrogen, or a substituted or unsubstituted alkyl, cycloalkyl, alkenyl, alkynyl, aryl aralkyl, heterocyclyl or heterocyclylalkyl group as defined herein.

The term “enamine” refers to —C(R⁹⁴)═C(R⁹⁵)NR⁹⁶R⁹⁷ and —NR⁹⁴C(R⁹⁵)═C(R⁹⁶)R⁹⁷, wherein R⁹⁴, R⁹⁵, R⁹⁶ and R⁹⁷ are each independently hydrogen, a substituted or unsubstituted alkyl, cycloalkyl, alkenyl, alkynyl, aryl aralkyl, heterocyclyl or heterocyclylalkyl group as defined herein.

The term “halogen” or “halo” as used herein refers to bromine, chlorine, fluorine, or iodine. In some embodiments, the halogen is fluorine. In other embodiments, the halogen is chlorine or bromine.

The term “hydroxyl” as used herein can refer to —OH or its ionized form, —O⁻.

The term “imide” refers to —C(O)NR⁹⁸C(O)R⁹⁹, wherein R⁹⁸ and R⁹⁹ are each independently hydrogen, or a substituted or unsubstituted alkyl, cycloalkyl, alkenyl, alkynyl, aryl aralkyl, heterocyclyl or heterocyclylalkyl group as defined herein.

The term “imine” refers to —CR⁹⁰⁰(NR⁹⁰¹) and —N(CR⁹⁰⁰R⁹⁰¹) groups, wherein R⁹⁰⁰ and R⁹⁰¹ are independently at each occurrence hydrogen or a substituted or unsubstituted alkyl, cycloalkyl, alkenyl, alkynyl, aryl aralkyl, heterocyclyl or heterocyclylalkyl group as defined herein, with the proviso that R⁹⁰⁰ and R⁹⁰¹ are not both simultaneously hydrogen.

The term “nitro” as used herein refers to an —NO₂ group.

As used herein, the “administration” of an agent, drug, peptide, or peptoid to a subject includes any route of introducing or delivering to a subject a compound to perform its intended function. Administration can be carried out by any suitable route, including orally, intranasally, parenterally (intravenously, intramuscularly, intraperitoneally, or subcutaneously), or topically. Administration includes self-administration and the administration by another.

As used herein, the term “amino acid” includes naturally-occurring amino acids and synthetic amino acids, as well as amino acid analogues and amino acid mimetics that function in a manner similar to the naturally-occurring amino acids. Naturally-occurring amino acids are those encoded by the genetic code, as well as those amino acids that are later modified, e.g., hydroxyproline, γ-carboxyglutamate, and O-phosphoserine. Amino acid analogues refer to compounds that have the same basic chemical structure as a naturally-occurring amino acid, i.e., an α-carbon that is bound to a carboxyl group, an amino group, and an R group, e.g., homoserine, norleucine, methionine sulfoxide, methionine methyl sulfonium. Such analogues have modified R groups (e.g., norleucine) or modified peptoid backbones, but retain the same basic chemical structure as a naturally-occurring amino acid. Amino acid mimetics refer to chemical compounds that have a structure that is different from the general chemical structure of an amino acid, but that functions in a manner similar to a naturally-occurring amino acid. Amino acids can be referred to herein by either their commonly known three letter symbols or by the one-letter symbols recommended by the IUPAC-IUB Biochemical Nomenclature Commission.

As used herein, “chemically bonded” refers to an attachment by means of a covalent bond. “Physically bonded” refers to an attachment by means of a physical interaction (non covalent bond). Examples are but not limited to H-bonds, pi stacking electrostatic interactions, matrices, salts, co-crystals, occlusion, solvates, hydrates, Van der Waal forces and London dispersion forces.

As used herein, the term “effective amount” refers to a quantity sufficient to achieve a desired therapeutic and/or prophylactic effect, e.g., an amount which results in the prevention of, or a decrease in a disease or disorder or one or more signs or symptoms associated with a disease or disorder. In the context of therapeutic or prophylactic applications, the amount of a composition administered to the subject will depend on the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compounds may be administered to a subject having one or more signs or symptoms of a disease or disorder.

As used herein, an “isolated” or “purified” polypeptide, peptide, or peptoid is substantially free of cellular material or other contaminating polypeptides from the cell or tissue source from which the agent is derived, or substantially free from chemical precursors or other chemicals when chemically synthesized. For example, an isolated neutral-cationic peptoid would be free of materials that would interfere with diagnostic or therapeutic uses of the agent. Such interfering materials may include enzymes, hormones, other proteinaceous and nonproteinaceous solutes, chemical precursors, and chemical impurities.

As used herein, the term “non-naturally-occurring” refers to a composition which is not found in this form in nature. A non-naturally-occurring composition can be derived from a naturally-occurring composition, e.g., as non-limiting examples, via purification, isolation, concentration, chemical modification (e.g., addition or removal of a chemical group), and/or, in the case of mixtures, addition or removal of ingredients or compounds. Alternatively, a non-naturally-occurring composition can comprise or be derived from a non-naturally-occurring combination of naturally-occurring compositions. Thus, a non-naturally-occurring composition can comprise a mixture of purified, isolated, modified and/or concentrated naturally-occurring compositions, and/or can comprise a mixture of naturally-occurring compositions in forms, concentrations, ratios and/or levels of purity not found in nature.

As used herein, the term “net charge” refers to the balance of the number of positive charges and the number of negative charges carried by the peptoid monomers present in the neutral-cationic peptoids of the present technology. In this specification, it is understood that net charges are measured at physiological pH. Peptoid monomers that are positively charged at physiological pH include ηlysine, ηarginine, and ηhistidine. Peptoid monomers that are negatively charged at physiological pH include ηaspartic acid and ηglutamic acid. Naturally occurring amino acids that are positively charged at physiological pH include L-lysine, L-arginine, and L-histidine. Naturally occurring amino acids that are negatively charged at physiological pH include L-aspartic acid and L-glutamic acid.

As used herein, the terms “polypeptide,” “peptide,” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art.

As used herein, “prevention” or “preventing” of a disorder or condition refers to one or more compounds that, in a statistical sample, reduces the occurrence of the disorder or condition in the treated sample relative to an untreated control sample, or delays the onset of one or more symptoms of the disorder or condition relative to the untreated control sample.

As used herein, the term “protecting group” refers to a chemical group that exhibits the following characteristics: 1) reacts selectively with the desired functionality in good yield to give a protected substrate that is stable to the projected reactions for which protection is desired; 2) is selectively removable from the protected substrate to yield the desired functionality; and 3) is removable in good yield by reagents compatible with the other functional group(s) present or generated in such projected reactions. Examples of suitable protecting groups can be found in Greene et al. (1991) Protective Groups in Organic Synthesis, 3rd Ed. (John Wiley & Sons, Inc., New York), incorporated herein by reference in its entirety for any and all purposes. Amino protecting groups include, but are not limited to, mesitylenesulfonyl (Mts), benzyloxycarbonyl (CBz or Z), t-butyloxycarbonyl (Boc), t-butyldimethylsilyl (TBS or TBDMS), 9-fluorenylmethyloxycarbonyl (Fmoc), acetyl (Ac), trifluoroacetyl, tosyl, benzenesulfonyl, 2-pyridyl sulfonyl, or suitable photolabile protecting groups such as 6-nitroveratryloxy carbonyl (Nvoc), nitropiperonyl, pyrenylmethoxycarbonyl, nitrobenzyl, α-,α-dimethyldimethoxybenzyloxycarbonyl (DDZ), 5-bromo-7-nitroindolinyl, and the like, as well as phosphoryl protecting groups as exemplified by the following structure:

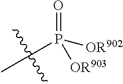

where R⁹⁰² and R⁹⁰³ are each independently hydrogen or a substituted or unsubstituted alkyl, aryl, heterocyclyl, heteroaryl group. Hydroxyl protecting groups include, but are not limited to, Fmoc, TBS, photolabile protecting groups (such as nitroveratryl oxymethyl ether (Nvom)), Mom (methoxy methyl ether), and Mem (methoxyethoxy methyl ether), NPEOC (4-nitrophenethyloxycarbonyl) and NPEOM (4-nitrophenethyloxymethyloxycarbonyl).

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, the terms “subject,” “individual,” or “patient” can be an individual organism, a vertebrate, a mammal, or a human.

As used herein, a “therapeutically effective amount” of a compound refers to compound levels in which the physiological effects of a disease or disorder are, at a minimum, ameliorated. A therapeutically effective amount can be given in one or more administrations. The amount of a compound which constitutes a therapeutically effective amount will vary depending on the compound, the disorder and its severity, and the general health, age, sex, body weight and tolerance to drugs of the subject to be treated, but can be determined routinely by one of ordinary skill in the art.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder.

It is also to be appreciated that the various modes of treatment or prevention of medical diseases and conditions as described are intended to mean “substantial,” which includes total but also less than total treatment or prevention, and wherein some biologically or medically relevant result is achieved. The treatment may be a continuous prolonged treatment for a chronic disease or a single, or few time administrations for the treatment of an acute condition.

### II. Neutral-Cationic Peptoids of the Present Technology

Peptoids are a polymer including two or more N-substituted glycines joined to each other by amide bonds. Monomers that make up the peptoid, such as N-substituted glycine, are termed “peptoid monomers.” Peptoids are resistant to proteolysis, a distinct advantage for therapeutic applications where proteolysis is a concern.

Peptoid monomers possessing the same side chains as known α-amino acids are represented by a “η” at the beginning of the known α-amino acid name (e.g., names recommended by the IUPAC-IUB Biochemical Nomenclature Commission) to indicate the side chain is on a glycine nitrogen atom, and peptoid nomenclature as used herein and understood by one of ordinary skill in the art is similar to peptide nomenclature. For example, the peptoid monomer N-methylglycine may be referred to herein as “ηalanine,” “ηAla” per the corresponding 3-letter abbreviation for alanine, or “ηA” per the corresponding 1-letter abbreviation for alanine. Glycine (i.e., aminoethanoic acid) does not have a side chain, therefore the recitation of “glycine” or “Gly” herein is therefore equivalent with “ηglycine” or “ηGly.” Accordingly, the peptoid ηTyr-ηArg-ηPhe-ηLys-NH₂ will be understood to have the following structure:

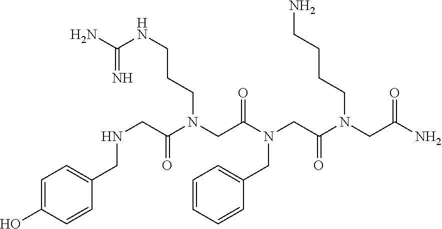 [SMILES: N=C(N)NCCCN(CC(=O)N(CC(=O)N(CCCCN)CC(N)=O)Cc1ccccc1)C(=O)CNCc1ccc(O)cc1]

Similarly, η2′,6′-dimethyltyrosine (“2′,6′-dimethyl-ηTyr” or “η2′,6′-Dmt”; the peptoid analogue of 2′,6′-dimethyltyrosine) will be understood to have the following structure:

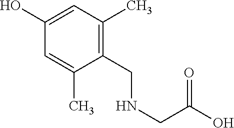

The neutral-cationic peptoid may also include one, two, or three non-glycine amino acid monomers, such as naturally or non-naturally occurring amino acids, so long as the peptoid contains at least one peptoid monomer. As used herein, the term “amino acid” is used to refer to any organic molecule that contains at least one amino group and at least one carboxyl group. Examples include, but are not limited to, diaminobutyric acid (Dab), diaminopropionic acid (Dap), β-dansyl-L-α,β-diaminopropionic acid (“(dns)Dap”), and β-anthraniloyl-L-α,β-diaminopropionic acid (“(atn)Dap”). In embodiments including an amino acid, it may be the amino group is at the α position relative to the carboxyl group (an “α-amino acid”). Naturally occurring amino acids include, for example, the twenty most common levorotatory (L,) amino acids normally found in mammalian proteins, i.e., alanine (Ala), arginine (Arg), asparagine (Asn), aspartic acid (Asp), cysteine (Cys), glutamine (Gln), glutamic acid (Glu), glycine (Gly), histidine (His), isoleucine (Ile), leucine (Leu), lysine (Lys), methionine (Met), phenylalanine (Phe), proline (Pro), serine (Ser), threonine (Thr), tryptophan, (Trp), tyrosine (Tyr), and valine (Val) Other naturally occurring amino acids include, for example, amino acids that are synthesized in metabolic processes not associated with protein synthesis. For example, the amino acids ornithine and citrulline are synthesized in mammalian metabolism during the production of urea. Non-naturally occurring amino acids may be (L-), dextrorotatory (D-), or mixtures thereof. Non-naturally occurring amino acids are those amino acids that typically are not synthesized in normal metabolic processes in living organisms, and do not naturally occur in proteins. For example, in some embodiments of the peptoids of the present technology, proline is an amino acid monomer of the neutral-cationic peptoid; in some embodiments of the present technology, an amino acid monomer of the present technology is a D-α-amino acid. In still other embodiments, an amino acid monomer is 2,6-dimethyl-Phe or 2,6-dimethyl-Tyr.

The neutral-cationic peptoids of the present technology preferably include a minimum of three peptoid monomers, covalently joined by amide bonds.

The maximum number of peptoid monomers present in the neutral-cationic peptoids of the present technology is about twenty peptoid monomers covalently joined by amide bonds. In some embodiments, the total number of peptoid monomers is about twelve. In some embodiments, the total number of peptoid monomers is about nine. In some embodiments, the total number of peptoid monomers is about six. In some embodiments, the total number of peptoid monomers is four.

In some aspects, the present technology provides a neutral-cationic peptoid or a pharmaceutically acceptable salt thereof such as acetate salt, tartrate salt, fumarate salt, hydrochloride salt, or trifluoroacetate salt. In some embodiments, the peptoid comprises at least one net positive charge; a minimum of three peptoid monomers; a maximum of about twenty peptoid monomers;

a relationship between the minimum number of net positive charges (pₘ) and the total number of peptoid monomer residues (r) wherein 3 pₘ is the largest number that is less than or equal to r+1; and

a relationship between the minimum number of aromatic groups (a) and the total number of net positive charges (pₜ) wherein 2a is the largest number that is less than or equal to pₜ+1, except that when a is 1, pₜ may also be 1.

In some embodiments, the peptoid is defined by Formula I:

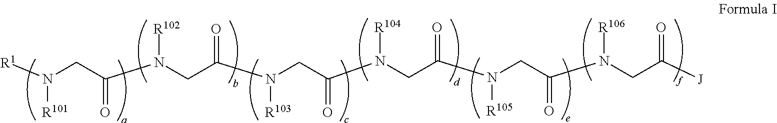

wherein:
J is —N(R³)(R⁴) or —O—R⁵;
R¹⁰¹ is

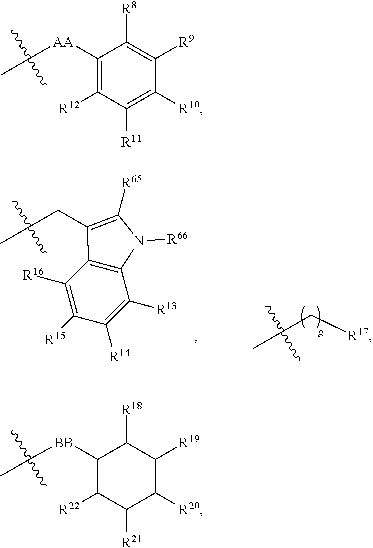

or R²;
R¹⁰² is

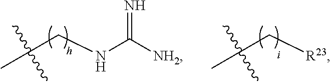

or hydrogen, or optionally R² if a is 0;
R¹⁰³ is

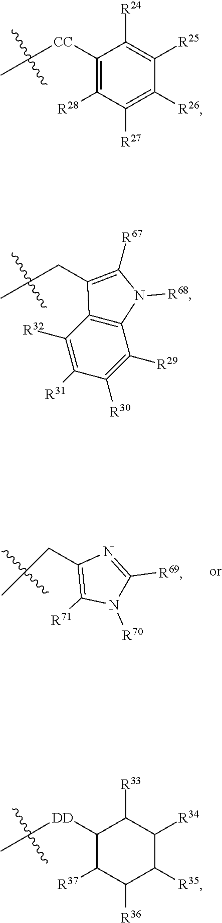

or optionally R² if a and b are each 0;
R¹⁰⁴ is

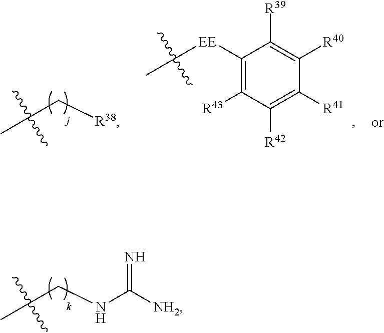

or optionally R² if a, b, and c are each 0;
R¹⁰⁵ is

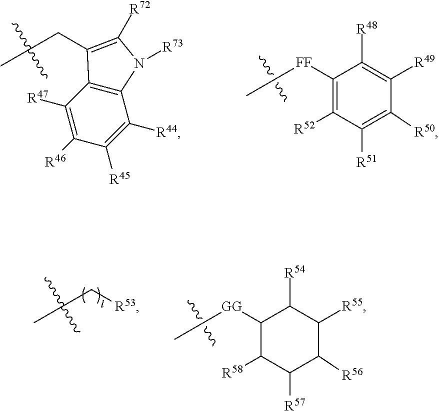

or hydrogen, or optionally R² if a, b, c, and d are each 0;
R¹⁰⁶ is

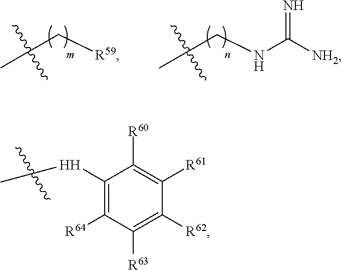

or hydrogen;
   wherein
      R¹, R², R³, R⁴, and R⁵ are each independently a hydrogen or substituted or unsubstituted C₁-C₆ alkyl, C₂-C₆ alkenyl, C₂-C₆ alkynyl, saturated or unsaturated cycloalkyl, cycloalkylalkyl, aryl, aralkyl, 5- or 6-membered saturated or unsaturated heterocylyl, heteroaryl, or amino protecting group; or R¹ and R² together or R³ and R⁴ together form a 3, 4, 5, 6, 7, or 8 membered substituted or unsubstituted heterocycyl ring;
      R⁸, R⁹, R¹⁰, R¹¹, R¹², R¹³, R¹⁴, R¹⁵, R¹⁶, R¹⁸, R¹⁹, R²⁰, R²¹, R²², R²⁴, R²⁵, R²⁶, R²⁷, R²⁸, R²⁹, R³⁰, R³¹, R³², R³³, R³⁴, R³⁵, R³⁶, R³⁷, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵⁴, R⁵⁵, R⁵⁶, R⁵⁷, R⁵⁸, R⁶⁰, R⁶¹, R⁶², R⁶³, R⁶⁴, R⁶⁵, R⁶⁷, R⁶⁹, R⁷¹, and R⁷² are each independently a hydrogen, amino, amido, —NO₂, —CN, —OR^{a}, —SR^{a}, —NR^{a}R^{a}, —F, —Cl, —Br, —I, or a substituted or unsubstituted C₁-C₆ alkyl, C₁-C₆ alkoxy, —C(O)-alkyl, —C(O)-aryl, —C(O)-aralkyl, —C(O)₂R^{a}, C₁-C₄ alkylamino, C₁-C₄ dialkylamino, or perhaloalkyl group;
      R⁶⁶, R⁶⁸, R⁷⁰, and R⁷³ are each independently a hydrogen or substituted or unsubstituted C₁-C₆ alkyl group;
      R¹⁷, R²³, R³⁸, R⁵³, and R⁵⁹ are each independently a hydrogen, —OR^{a}, —SR^{a}, —NR^{a}R^{a}, —NR^{a}R^{b}, —CO₂R^{a}, —(CO)NR^{a}R^{a}, —NR^{a}(CO)R^{a}, —NR^{a}C(NH)NH₂, —NR^{a}-dansyl, enamine, imine, or a substituted or unsubstituted alkyl, heterocyclyl, aryl, heteroaryl, or aralkyl group;
      AA, BB, CC, DD, EE, FF, GG, and HH are each independently absent, —NH(CO)—, or —CH₂—;
      R^{a} at each occurrence is independently a hydrogen or a substituted or unsubstituted C₁-C₆ alkyl group;
      R^{b} at each occurrence is independently a C₁-C₆ alkylene-NR^{a}-dansyl or C₁-C₆ alkylene-NR^{a}-anthraniloyl group;
      a, b, c, d, e, and f are each independently 0 or 1,
         with the proviso that a+b+c+d+e+f≥2; and
      g, h, i, j, k, l, m, and n are independently at each occurrence 1, 2, 3, 4, or 5.

In any embodiment herein of a peptoid of Formula I, it may be that
R¹, R², R³, R⁴, and R⁵ are each independently a hydrogen or substituted or unsubstituted C₁-C₆ alkyl group;
R⁸, R¹², R¹⁸, R²², R²⁴, R²⁸, R³³, R³⁷, R³⁹, R⁴³, R⁴⁸, R⁵², R⁵⁴, R⁵⁸, R⁶⁰, and R⁶⁴ are each independently a hydrogen or methyl group;
R¹⁰, R²⁰, R²⁶, R³⁵, R⁴¹, R⁵⁰, R⁵⁶, and R⁶² are each independently a hydrogen or —OR^{a};
R⁹, R¹¹, R¹⁹, R²¹, R²⁵, R²⁷, R³⁴, R³⁶, R⁴⁰, R⁴², R⁴⁹, R⁵¹, R⁵⁵, R⁵⁷, R⁶¹, R⁶³, R⁶⁵, R⁶⁶, R⁶⁷, R⁶⁸, R⁶⁹, R⁷⁰, R⁷¹, R⁷², and R⁷³ are each a hydrogen;
R¹⁷, R²³, R³⁸, R⁵³, and R⁵⁹ are each independently a hydrogen, —OH, —SH, —SCH₃, —NH₂, —NHR^{b}, —CO₂H, —(CO)NH₂, —NH(CO)H, or —NH-dansyl group;
AA, BB, CC, DD, EE, FF, GG, and HH are each independently absent or —CH₂—;
R^{b} at each occurrence is independently an ethylene-NH-dansyl or ethylene-NH-anthraniloyl group.

In any embodiment of Formula I, it may be at least one of R¹⁰¹, R¹⁰², R¹⁰⁴, R¹⁰⁵, and R¹⁰⁶ is a basic group, as defined above, and at least one of R¹⁰¹, R¹⁰³, R¹⁰⁴, R¹⁰⁵, and R¹⁰⁶ is a neutral group as defined above. In such embodiments, it may be that the neutral group is an aromatic, heterocyclic or cycloalkyl group as defined above. In any embodiment of Formula I, it may be the peptoid includes at least one cationic residue such as ηarginine and at least one neutral residue such as η2′,6′-dimethyltyrosine, ηtyrosine, or ηphenylalanine. In any embodiment of Formula I, it may be that R¹⁰¹ is an alkylguanidinium group.

In some embodiments, the peptoid of the present technology is selected from the peptoids shown in Tables A or B.

TABLE A
ηTyr-ηArg-ηPhe-ηLys-NH₂
ηTyr-ηHar-ηPhe-ηLys-NH₂
ηTyr-ηAgb-ηPhe-ηLys-NH₂
ηArg-ηDmt-ηLys-ηPhe-NH₂
ηHar-ηDmt-ηLys-ηPhe-NH₂
ηAgb-ηDmt-ηLys-ηPhe-NH₂
ηArg-ηDmt-ηOrn-ηPhe-NH₂
ηArg-ηDmt-ηOrn-ηOrn-ηPhe-NH₂
ηOrn-ηDmt-ηOrn-ηPhe-NH₂
ηArg-ηDmt-ηPhe-ηLys-NH₂
ηHar-ηDmt-ηPhe-ηLys-NH₂
ηAgb-ηDmt-ηPhe-ηLys-NH₂
ηArg-ηPhe-ηLys-ηDmt-NH₂
ηHar-ηPhe-ηLys-ηDmt-NH₂
ηAgb-ηPhe-ηLys-ηDmt-NH₂
ηArg-ηPhe-ηDmt-ηLys-NH₂
ηHar-ηPhe-ηDmt-ηLys-NH₂
ηAgb-ηPhe-ηDmt-ηLys-NH₂
ηArg-ηLys-ηDmt-ηPhe-NH₂
ηHar-ηLys-ηDmt-ηPhe-NH₂
ηAgb-ηLys-ηDmt-ηPhe-NH₂
ηArg-ηLys-ηPhe-ηDmt-NH₂
ηHar-ηLys-ηPhe-ηDmt-NH₂
ηAgb-ηLys-ηPhe-ηDmt-NH₂
ηArg-ηDmt-ηLys-ηPhe-ηCys-NH₂
ηHar-ηDmt-ηLys-ηPhe-ηCys-NH₂
ηAgb-ηDmt-ηLys-ηPhe-ηCys-NH₂
ηPhe-ηLys-ηDmt-ηArg-NH₂
ηPhe-ηLys-ηDmt-ηHar-NH₂
ηPhe-ηLys-ηDmt-ηAgb-NH₂
ηPhe-ηThys-ηArg-ηDmt-NH₂
ηPhe-ηLys-ηHar-ηDmt-NH₂
ηPhe-ηLys-ηAgb-ηDmt-NH₂
ηPhe-ηArg-ηThe-ηThys-NH₂
ηPhe-ηHar-ηPhe-ηLys-NH₂
ηPhe-ηAgb-ηPhe-ηLys-NH₂
ηPhe-ηArg-ηPhe-ηLys-ηCys-NH₂
ηPhe-ηHar-ηPhe-ηLys-ηCys-NH₂
ηPhe-ηAgb-ηPhe-ηLys-ηCys-NH₂
ηPhe-ηArg-ηPhe-ηLys-ηSer-ηCys-NH₂
ηPhe-ηHar-ηPhe-ηLys-ηSer-ηCys-NH₂
ηPhe-ηAgb-ηPhe-ηLys-ηSer-ηCys-NH₂
ηPhe-ηArg-ηPhe-ηLys-Gηy-ηCys-NH₂
ηPhe-ηHar-ηPhe-ηLys-Gηy-ηCys-NH₂
ηPhe-ηAgb-ηPhe-ηLys-Gηy-ηCys-NH₂
ηPhe-ηArg-ηDmt-ηLys-NH₂
ηPhe-ηHar-ηDmt-ηLys-NH₂
ηPhe-ηAgb-ηDmt-ηLys-NH₂
ηPhe-ηArg-ηDmt-ηThys-ηCys-NH₂
ηPhe-ηHar-ηDmt-ηLys-ηCys-NH₂
ηPhe-ηAgb-ηDmt-ηLys-ηCys-NH₂
ηPhe-ηArg-ηDmt-ηLys-ηSer-ηCys-NH₂
ηPhe-ηHar-ηDmt-ηLys-ηSer-ηCys-NH₂
ηPhe-ηAgb-ηDmt-ηLys-ηSer-ηCys-NH₂
ηPhe-ηArg-ηDmt-ηThys-Gηy-ηCys-NH₂
ηPhe-ηHar-ηDmt-ηLys-Gηy-ηCys-NH₂
ηPhe-ηAgb-ηDmt-ηLys-Gηy-ηCys-NH₂
ηPhe-ηArg-ηLys-ηDmt-NH₂
ηPhe-ηHar-ηLys-ηDmt-NH₂
ηPhe-ηAgb-ηLys-ηDmt-NH₂
ηPhe-ηDmt-ηArg-ηThys-NH₂
ηPhe-ηDmt-ηHar-ηLys-NH₂
ηPhe-ηDmt-ηAgb-ηLys-NH₂
ηPhe-ηDmt-ηLys-ηArg-NH₂
ηPhe-ηDmt-ηLys-ηHar-NH₂
ηPhe-ηDmt-ηLys-ηAgb-NH₂
ηLys-ηPhe-ηArg-ηDmt-NH₂
ηLys-ηPhe-ηHar-ηDmt-NH₂
ηLys-ηPhe-ηAgb-ηDmt-NH₂
ηLys-ηPhe-ηDmt-ηArg-NH₂
ηLys-ηPhe-ηDmt-ηHar-NH₂
ηLys-ηPhe-ηDmt-ηAgb-NH₂
ηLys-ηDmt-ηArg-ηThe-NH₂
ηLys-ηDmt-ηHar-ηPhe-NH₂
ηLys-ηDmt-ηAgb-ηPhe-NH₂
ηLys-ηDmt-ηPhe-ηArg-NH₂
ηLys-ηDmt-ηPhe-ηHar-NH₂
ηLys-ηDmt-ηPhe-ηAgb-NH₂
ηLys-ηArg-ηPhe-ηDmt-NH₂
ηLys-ηHar-ηPhe-ηDmt-NH₂
ηLys-ηAgb-ηPhe-ηDmt-NH₂
ηLys-ηArg-ηDmt-ηPhe-NH₂
ηLys-ηHar-ηDmt-ηPhe-NH₂
ηLys-ηAgb-ηDmt-ηPhe-NH₂
ηArg-ηDmt-ηArg-ηPhe-NH₂
ηArg-ηDmt-ηHar-ηPhe-NH₂
ηHar-ηDmt-ηHar-ηPhe-NH₂
ηAgb-ηDmt-ηHar-ηPhe-NH₂
ηArg-ηDmt-ηAgb-ηPhe-NH₂
ηHar-ηDmt-ηAgb-ηPhe-NH₂
ηAgb-ηDmt-ηAgb-ηPhe-NH₂
ηArg-ηDmt-ηArg-ηDmt-NH₂
ηArg-ηDmt-ηHar-ηDmt-NH₂
ηHar-ηDmt-ηHar-ηDmt-NH₂
ηAgb-ηDmt-ηHar-ηDmt-NH₂
ηArg-ηDmt-ηAgb-ηDmt-NH₂
ηHar-ηDmt-ηAgb-ηDmt-NH₂
ηAgb-ηDmt-ηAgb-ηDmt-NH₂
ηArg-ηDmt-ηArg-ηTyr-NH₂
ηArg-ηDmt-ηHar-ηTyr-NH₂
ηHar-ηDmt-ηHar-ηTyr-NH₂
ηAgb-ηDmt-ηHar-ηTyr-NH₂
ηArg-ηDmt-ηAgb-ηTyr-NH₂
ηHar-ηDmt-ηAgb-ηTyr-NH₂
ηAgb-ηDmt-ηAgb-ηTyr-NH₂
ηArg-ηDmt-ηArg-ηTrp-NH₂
ηArg-ηDmt-ηHar-ηTrp-NH₂
ηHar-ηDmt-ηHar-ηTrp-NH₂
ηAgb-ηDmt-ηHar-ηTrp-NH₂
ηArg-ηDmt-ηAgb-ηTrp-NH₂
ηHar-ηDmt-ηAgb-ηTrp-NH₂
ηAgb-ηDmt-ηAgb-ηTrp-NH₂
ηArg-ηDmt-ηOrn-ηTrp-NH₂
ηHar-ηDmt-ηOrn-ηTrp-NH₂
ηAgb-ηDmt-ηOrn-ηTrp-NH₂
ηTrp-ηArg-ηgyr-ηLys-NH₂
ηTrp-ηHar-ηTyr-ηLys-NH₂
ηTrp-ηAgb-ηTyr-ηLys-NH₂
ηTrp-ηArg-ηgrp-ηLys-NH₂
ηTrp-ηHar-ηTrp-ηLys-NH₂
ηTrp-ηAgb-ηTrp-ηLys-NH₂
ηTrp-ηArg-ηDmt-ηLys-NH₂
ηTrp-ηHar-ηDmt-ηLys-NH₂
ηTrp-ηAgb-ηDmt-ηLys-NH₂
ηArg-ηTrp-ηLys-ηPhe-NH₂
ηHar-ηTrp-ηLys-ηPhe-NH₂
ηAgb-ηTrp-ηLys-ηPhe-NH₂
ηArg-ηTrp-ηThe-ηLys-NH₂
ηHar-ηTrp-ηPhe-ηLys-NH₂
ηAgb-ηTrp-ηPhe-ηLys-NH₂
ηArg-ηTrp-ηLys-ηDmt-NH₂
ηHar-ηTrp-ηLys-ηDmt-NH₂
ηAgb-ηTrp-ηLys-ηDmt-NH₂
ηArg-ηTrp-ηDmt-ηLys-NH₂
ηHar-ηTrp-ηDmt-ηLys-NH₂
ηAgb-ηTrp-ηDmt-ηLys-NH₂
ηArg-ηLys-ηTrp-ηPhe-NH₂
ηHar-ηLys-ηTrp-ηPhe-NH₂
ηAgb-ηLys-ηTrp-ηPhe-NH₂
ηArg-ηLys-ηTrp-ηDmt-NH₂
ηHar-ηLys-ηTrp-ηDmt-NH₂
ηAgb-ηLys-ηTrp-ηDmt-NH₂
ηCha-ηArg-ηPhe-ηLys-NH₂
ηCha-ηHar-ηPhe-ηLys-NH₂
ηCha-ηAgb-ηPhe-ηLys-NH₂
ηAηa-ηArg-ηPhe-ηLys-NH₂
ηAηa-ηHar-ηPhe-ηLys-NH₂
ηAηa-ηAgb-ηPhe-ηLys-NH₂
η2',6'-Dmp-ηArg-η2',6'-Dmt-ηLys-NH₂
η2',6'-Dmp-ηHar-η2',6'-Dmt-ηLys-NH₂
η2',6'-Dmp-ηAgb-η2',6'-Dmt-ηLys-NH₂
η2',6'-Dmp-ηArg-ηPhe-ηLys-NH₂
η2',6'-Dmp-ηHar-ηPhe-ηLys-NH₂
η2',6'-Dmp-ηAgb-ηPhe-ηLys-NH₂
η2',6'-Dmt-ηArg-ηPhe-ηOrn-NH₂
η2',6'-Dmt-ηHar-ηPhe-ηOrn-NH₂
η2',6'-Dmt-ηAgb-ηPhe-ηOrn-NH₂
η2',6'-Dmt-ηArg-ηPhe-ηAhp-NH₂
η2',6'-Dmt-ηHar-ηPhe-ηAhp-NH₂
η2',6'-Dmt-ηAgb-ηPhe-ηAhp-NH₂
η2',6'-Dmt-ηArg-ηPhe-ηLys-NH₂
η2',6'-Dmt-ηHar-ηPhe-ηThys-NH₂
η2',6'-Dmt-ηAgb-ηPhe-ηLys-NH₂
η2',6'-Dmt-ηCit-ηPhe-ηLys-NH₂
ηArg-η2',6'-Dmt-ηLys-ηPhe-NH₂
ηHar-η2',6'-Dmt-ηLys-ηPhe-NH₂
ηAgb-η2',6'-Dmt-ηLys-ηPhe-NH₂
ηTyr-ηTrp-ηLys-NH₂
ηLys-ηArg-ηTyr-NH₂
ηLys-ηHar-ηTyr-NH₂
ηLys-ηAgb-ηTyr-NH₂
ηMet-ηTyr-ηArg-ηPhe-ηArg-NH₂
ηMet-ηTyr-ηHar-ηPhe-ηArg-NH₂
ηMet-ηTyr-ηHar-ηPhe-ηHar-NH₂
ηMet-ηTyr-ηHar-ηPhe-ηAgb-NH₂
ηMet-ηTyr-ηAgb-ηPhe-ηArg-NH₂
ηMet-ηTyr-ηAgb-ηPhe-ηHar-NH₂
ηMet-ηTyr-ηAgb-ηPhe-ηAgb-NH₂
ηMet-ηTyr-ηLys-ηPhe-ηArg
ηMet-ηTyr-ηLys-ηPhe-ηHar
ηMet-ηTyr-ηLys-ηPhe-ηAgb
ηPhe-ηArg-ηHis-ηAsp
ηPhe-ηHar-ηHis-ηAsp
ηPhe-ηAgb-ηHis-ηAsp
ηPhe-ηArg-η2',6'-Dmt-ηLys-NH₂
ηPhe-ηHar-η2',6'-Dmt-ηLys-NH₂
ηPhe-ηAgb-η2',6'-Dmt-ηLys-NH₂
ηPhe-ηArg-ηHis
ηPhe-ηHar-ηHis
ηPhe-ηAgb-ηHis
ηTrp-ηLys-ηTyr-ηArg-NH₂
ηTrp-ηLys-ηTyr-ηHar-NH₂
ηTrp-ηLys-ηTyr-ηAgb-NH₂
ηTyr-ηArg-ηPhe-ηLys-ηGηu-NH₂
ηTyr-ηHar-ηPhe-ηLys-ηGηu-NH₂
ηTyr-ηAgb-ηPhe-ηLys-ηGηu-NH₂
ηTyr-ηHis-ηGηy-ηMet
ηArg-ηTyr-ηLys-ηPhe-NH₂
ηHar-ηTyr-ηLys-ηPhe-NH₂
ηAgb-ηTyr-ηLys-ηPhe-NH₂
ηArg-ηDmt-ηLys-ηPhe-NH₂
ηHar-ηDmt-ηLys-ηhe-NH₂
ηAgb-ηDmt-ηLys-ηPhe-NH₂
ηArg-ηDmt-ηLys-ηPhe-NH₂
ηHar-ηDmt-ηLys-ηPhe-NH₂
ηAgb-ηDmt-ηLys-ηPhe-NH₂
ηPhe-ηArg-ηThe-ηThys-NH₂
ηPhe-ηHar-ηPhe-ηLys-NH₂
ηPhe-ηAgb-ηPhe-ηLys-NH₂
ηLys-ηPhe-ηArg-ηDmt-NH₂
ηLys-ηPhe-ηHar-ηDmt-NH₂
ηLys-ηPhe-ηAgb-ηDmt-NH₂
ηArg-ηArg-ηDmt-ηPhe-NH₂
ηArg-ηHar-ηDmt-ηPhe-NH₂
ηArg-ηAgb-ηDmt-ηPhe-NH₂
ηHar-ηArg-ηDmt-ηPhe-NH₂
ηHar-ηHar-ηDmt-ηPhe-NH₂
ηHar-ηAgb-ηDmt-ηPhe-NH₂
ηAgb-ηArg-ηDmt-ηPhe-NH₂
ηAgb-ηHar-ηDmt-ηPhe-NH₂
ηAgb-ηAgb-ηDmt-ηPhe-NH₂
ηDmt-ηPhe-ηArg-ηηLys-NH₂
ηDmt-ηPhe-ηHar-ηLys-NH₂
ηDmt-ηPhe-ηAgb-ηLys-NH₂
ηPhe-ηDmt-ηArg-ηThys-NH₂
ηPhe-ηDmt-ηHar-ηLys-NH₂
ηPhe-ηDmt-ηAgb-ηLys-NH₂
ηArg-ηDmt-ηLys-NH₂
ηHar-ηDmt-ηLys-NH₂
ηAgb-ηDmt-ηLys-NH₂
ηArg-ηDmt-ηPhe-NH₂
ηHar-ηDmt-ηPhe-NH₂
ηAgb-ηDmt-ηPhe-NH₂
ηArg-ηDmt-ηArg-NH₂
ηArg-ηDmt-ηHar-NH₂
ηArg-ηDmt-ηAgb-NH₂
ηHar-ηDmt-ηArg-NH₂
ηHar-ηDmt-ηHar-NH₂
ηHar-ηDmt-ηAgb-NH₂
ηAgb-ηDmt-ηArg-NH₂
ηAgb-ηDmt-ηHar-NH₂
ηAgb-ηDmt-ηAgb-NH₂
ηDmt-ηArg-NH₂
ηDmt-ηHar-NH₂
ηDmt-ηAgb-NH₂
ηArg-ηDmt-NH₂
ηHar-ηDmt-NH₂
ηAgb-ηDmt-NH₂
ηArg-ηTyr-ηLys-ηPhe-NH₂
ηHar-ηTyr-ηLys-ηPhe-NH₂
ηAgb-ηTyr-ηLys-ηPhe-NH₂
ηLys-ηPhe-ηArg-ηTyr-NH₂
ηLys-ηPhe-ηHar-ηTyr-NH₂
ηLys-ηPhe-ηAgb-ηTyr-NH₂
ηArg-ηArg-ηTyr-ηPhe-NH₂
ηArg-ηHar-ηTyr-ηPhe-NH₂
ηArg-ηAgb-ηTyr-ηPhe-NH₂
ηHar-ηArg-ηTyr-ηPhe-NH₂
ηHar-ηHar-ηTyr-ηPhe-NH₂
ηHar-ηAgb-ηTyr-ηPhe-NH₂
ηAgb-ηArg-ηTyr-ηPhe-NH₂
ηAgb-ηHar-ηTyr-ηPhe-NH₂
ηAgb-ηAgb-ηTyr-ηPhe-NH₂
ηTyr-ηPhe-ηArg-ηLys-NH₂
ηTyr-ηPhe-ηHar-ηLys-NH₂
ηTyr-ηPhe-ηAgb-ηLys-NH₂
ηPhe-ηTyr-ηArg-ηLys-NH₂
ηPhe-ηTyr-ηHar-ηLys-NH₂
ηPhe-ηTyr-ηAgb-ηLys-NH₂
ηArg-ηTyr-ηLys-NH₂
ηHar-ηTyr-ηLys-NH₂
ηAgb-ηTyr-ηLys-NH₂
ηArg-ηTyr-ηPhe-NH₂
ηHar-ηTyr-ηPhe-NH₂
ηAgb-ηTyr-ηPhe-NH₂
ηArg-ηTyr-ηηArg-NH₂
ηArg-ηTyr-ηHar-NH₂
ηArg-ηTyr-ηAgb-NH₂
ηHar-ηTyr-ηArg-NH₂
ηHar-ηTyr-ηHar-NH₂
ηHar-ηTyr-ηAgb-NH₂
ηAgb-ηTyr-ηArg-NH₂
ηAgb-ηTyr-ηHar-NH₂
ηAgb-ηTyr-ηAgb-NH₂
ηTyr-ηArg-NH₂
ηTyr-ηHar-NH₂
ηTyr-ηAgb-NH₂
ηArg-ηTyr-NH₂
ηHar-ηTyr-NH₂
ηAgb-ηTyr-NH₂
ηDmt-ηLys-ηPhe-NH₂
ηLys-ηDmt-ηArg-NH₂
ηLys-ηDmt-ηHar-NH₂
ηLys-ηDmt-ηAgb-NH₂
ηPhe-ηLys-ηDmt-NH₂
ηArg-ηPhe-ηLys-NH₂
ηHar-ηPhe-ηLys-NH₂
ηAgb-ηPhe-ηLys-NH₂
ηArg-ηCha-ηLys-NH₂
ηHar-ηCha-ηLys-NH₂
ηAgb-ηCha-ηLys-NH₂
ηArg-ηTrp-ηLys-NH₂
ηHar-ηTrp-ηLys-NH₂
ηAgb-ηTrp-ηLys-NH₂
ηDmt-ηLys-ηPhe-NH₂
ηDmt-ηLys-NH₂
ηLys-ηPhe-NH₂
ηArg-ηCha-ηLys-ηCha-NH₂
ηHar-ηCha-ηLys-ηCha-NH₂
ηAgb-ηCha-ηLys-ηCha-NH₂
ηNle-ηDmt-ηAhp-ηPhe-NH₂
ηNle-ηCha-ηAhp-ηCha-NH₂
ηArg-ηDmt-ηLys-NH₂
ηHar-ηDmt-ηLys-NH₂
ηAgb-ηDmt-ηLys-NH₂
ηArg-ηDmt-ηLys-ηPhe-NH₂
ηHar-ηDmt-ηLys-ηPhe-NH₂
ηAgb-ηDmt-ηLys-ηPhe-NH₂
ηLys-ηTrp-ηηArg-NH₂
ηLys-ηTrp-ηHar-NH₂
ηLys-ηTrp-ηAgb-NH₂
H-ηLys-ηPhe-ηArg-ηDmt-NH₂
H-ηLys-ηPhe-ηHar-ηDmt-NH₂
H-ηLys-ηPhe-ηAgb-ηDmt-NH₂
H-ηArg-ηLys-ηDmt-ηPhe-NH₂
H-ηHar-ηLys-ηDmt-ηPhe-NH₂
H-ηAgb-ηLys-ηDmt-ηPhe-NH₂
H-ηArg-ηLys-ηPhe-ηDmt-NH₂
H-ηHar-ηLys-ηPhe-ηDmt-NH₂
H-ηAgb-ηLys-ηPhe-ηDmt-NH₂
H-ηArg-ηArg-ηDmt-ηPhe-NH₂
H-ηArg-ηHar-ηDmt-ηPhe-NH₂
H-ηArg-ηAgb-ηDmt-ηPhe-NH₂
H-ηHar-ηArg-ηDmt-ηPhe-NH₂
H-ηHar-ηHar-ηDmt-ηPhe-NH₂
H-ηHar-ηAgb-ηDmt-ηPhe-NH₂
H-ηAgb-ηArg-ηDmt-ηPhe-NH₂
H-ηAgb-ηHar-ηDmt-ηPhe-NH₂
H-ηAgb-ηAgb-ηDmt-ηPhe-NH₂
H-ηArg-ηDmt-ηPhe-ηLys-NH₂
H-ηHar-ηDmt-ηPhe-ηLys-NH₂
H-ηAgb-ηDmt-ηPhe-ηLys-NH₂
H-ηArg-ηPhe-ηDmt-ηLys-NH₂
H-ηHar-ηPhe-ηDmt-ηLys-NH₂
H-ηAgb-ηPhe-ηDmt-ηLys-NH₂
H-ηDmt-ηPhe-ηArg-ηLys-NH₂
H-ηDmt-ηPhe-ηHar-ηLys-NH₂
H-ηDmt-ηPhe-ηAgb-ηLys-NH₂
H-ηPhe-ηDmt-ηArg-ηLys-NH₂
H-ηPhe-ηDmt-ηHar-ηLys-NH₂
H-ηPhe-ηDmt-ηAgb-ηLys-NH₂
H-ηArg-ηDmt-ηLys-NH₂
H-ηHar-ηDmt-ηLys-NH₂
H-ηAgb-ηDmt-ηLys-NH₂
H-ηArg-ηDmt-ηLys-ηPhe-NH₂
H-ηHar-ηDmt-ηLys-ηPhe-NH₂
H-ηAgb-ηDmt-ηLys-ηPhe-NH₂
H-ηArg-ηDmt-ηLys-ηPhe-NH₂
H-ηHar-ηDmt-ηLys-ηPhe-NH₂
H-ηAgb-ηDmt-ηLys-ηPhe-NH₂
H-ηArg-ηDmt-ηPhe-NH₂
H-ηHar-ηDmt-ηPhe-NH₂
H-ηAgb-ηDmt-ηPhe-NH₂
H-ηDmt-ηArg-NH₂
H-ηDmt-ηHar-NH₂
H-ηDmt-ηAgb-NH₂
H-ηPhe-ηArg-ηPhe-ηLys-NH₂
H-ηPhe-ηHar-ηPhe-ηLys-NH₂
H-ηPhe-ηAgb-ηPhe-ηLys-NH₂
H-ηArg-ηDmt-ηLys-ηPhe-NH₂
H-ηHar-ηDmt-ηLys-ηPhe-NH₂
H-ηAgb-ηDmt-ηLys-ηPhe-NH₂
H-ηArg-ηCha-ηLys-NH₂
H-ηHar-ηCha-ηLys-NH₂
H-ηAgb-ηCha-ηLys-NH₂
H-ηArg-ηCha-ηLys-ηCha-NH₂
H-ηHar-ηCha-ηLys-ηCha-NH₂
H-ηAgb-ηCha-ηLys-ηCha-NH₂
H-ηArg-ηDmt-ηLys-NH₂
H-ηHar-ηDmt-ηLys-NH₂
H-ηAgb-ηDmt-ηLys-NH₂
H-ηArg-ηDmt-ηArg-NH₂
H-ηArg-ηDmt-ηHar-NH₂
H-ηArg-ηDmt-ηAgb-NH₂
H-ηHar-ηDmt-ηArg-NH₂
H-ηHar-ηDmt-ηHar-NH₂
H-ηHar-ηDmt-ηAgb-NH₂
H-ηAgb-ηDmt-ηArg-NH₂
H-ηAgb-ηDmt-ηHar-NH₂
H-ηAgb-ηDmt-ηAgb-NH₂
H-ηDmt-ηArg-NH₂
H-ηDmt-ηHar-NH₂
H-ηDmt-ηAgb-NH₂
H-ηArg-ηDmt-NH₂
H-ηHar-ηDmt-NH₂
H-ηAgb-ηDmt-NH₂
ηArg-ηArg-ηDmt-ηPhe
ηArg-ηHar-ηDmt-ηPhe
ηArg-ηAgb-ηDmt-ηPhe
ηHar-ηArg-ηDmt-ηPhe
ηHar-ηHar-ηDmt-ηPhe
ηHar-ηAgb-ηDmt-ηPhe
ηAgb-ηArg-ηDmt-ηPhe
ηAgb-ηHar-ηDmt-ηPhe
ηAgb-ηAgb-ηDmt-ηPhe
ηArg-ηCha-ηLys
ηHar-ηCha-ηLys
ηAgb-ηCha-ηLys
ηArg-ηDmt
ηHar-ηDmt
ηAgb-ηDmt
ηArg-ηDmt-ηArg
ηArg-ηDmt-ηHar
ηArg-ηDmt-ηAgb
ηHar-ηDmt-ηArg
ηHar-ηDmt-ηHar
ηHar-ηDmt-ηAgb
ηAgb-ηDmt-ηArg
ηAgb-ηDmt-ηHar
ηAgb-ηDmt-ηAgb
ηArg-ηDmt-ηLys
ηHar-ηDmt-ηLys
ηAgb-ηDmt-ηLys
ηArg-ηDmt-ηLys-ηPhe
ηHar-ηDmt-ηLys-ηPhe
ηAgb-ηDmt-ηLys-ηPhe
ηArg-ηDmt-ηLys-ηPhe-ηCys
ηHar-ηDmt-ηLys-ηPhe-ηCys
ηAgb-ηDmt-ηLys-ηPhe-ηCys
ηArg-ηDmt-ηPhe
ηHar-ηDmt-ηPhe
ηAgb-ηDmt-ηPhe
ηArg-ηDmt-ηPhe-ηLys
ηHar-ηDmt-ηPhe-ηLys
ηAgb-ηDmt-ηPhe-ηLys
ηArg-ηLys-ηDmt-ηPhe
ηHar-ηLys-ηDmt-ηPhe
ηAgb-ηLys-ηDmt-ηPhe
ηArg-ηLys-ηPhe-ηDmt
ηHar-ηLys-ηPhe-ηDmt
ηAgb-ηLys-ηPhe-ηDmt
ηArg-ηPhe-ηDmt-ηLys
ηHar-ηPhe-ηDmt-ηLys
ηAgb-ηPhe-ηDmt-ηLys
ηArg-ηPhe-ηLys
ηHar-ηPhe-ηLys
ηAgb-ηPhe-ηLys
ηArg-ηTrp-ηLys
ηHar-ηTrp-ηLys
ηAgb-ηTrp-ηLys
ηArg-ηTyr-ηLys
ηHar-ηTyr-ηLys
ηAgb-ηTyr-ηLys
ηArg-ηTyr-ηLys-ηPhe
ηHar-ηTyr-ηLys-ηPhe
ηAgb-ηTyr-ηLys-ηPhe
ηArg-ηDmt-ηLys-ηPhe-NH₂
ηHar-ηDmt-ηLys-ηPhe-NH₂
ηAgb-ηDmt-ηLys-ηPhe-NH₂
ηOrn-ηDmt-ηOrn-ηPhe-NH₂
ηArg-ηDmt-ηLys-NH₂
ηHar-ηDmt-ηLys-NH₂
ηAgb-ηDmt-ηLys-NH₂
ηArg-ηDmt-ηLys-ηPhe-ηCys
ηHar-ηDmt-ηLys-ηPhe-ηCys
ηAgb-ηDmt-ηLys-ηPhe-ηCys
ηArg-ηDmt-ηLys-ηPhe-NH₂
ηHar-ηDmt-ηLys-ηPhe-NH₂
ηAgb-ηDmt-ηLys-ηPhe-NH₂
ηDmt-ηArg
ηDmt-ηHar
ηDmt-ηAgb
ηDmt-ηLys
ηDmt-ηLys-ηPhe
ηDmt-ηPhe-ηArg-ηLys
ηDmt-ηPhe-ηHar-ηLys
ηDmt-ηPhe-ηAgb-ηLys
H-ηArg-ηDmt-ηLys-ηPhe-NH₂
H-ηHar-ηDmt-ηLys-ηPhe-NH₂
H-ηAgb-ηDmt-ηLys-ηPhe-NH₂
H-ηArg-η2,6-dichlorotyrosine-ηLys-ηPhe-NH₂
H-ηHar-η2,6-dichlorotyrosine-ηLys-ηPhe-NH₂
H-ηAgb-η2,6-dichlorotyrosine-ηLys-ηPhe-NH₂
H-ηArg-η2,6-difluorotyrosine-ηLys-ηPhe-NH₂
H-ηHar-η2,6-difluorotyrosine-ηLys-ηPhe-NH₂
H-ηAgb-η2,6-difluorotyrosine-ηLys-ηPhe-NH₂
H-ηArg-η2,6-dimethylphenylalanine-ηLys-ηPhe-NH₂
H-ηHar-η2,6-dimethylphenylalanine-ηLys-ηPhe-NH₂
H-ηAgb-η2,6-dimethylphenylalanine-ηLys-ηPhe-NH₂
H-ηArg-η4-methoxy-2,6-dimethylphenylalanine-ηLys-ηPhe-NH₂
H-ηHar-η4-methoxy-2,6-dimethylphenylalanine-ηLys-ηPhe-NH₂
H-ηAgb-η4-methoxy-2,6-dimethylphenylalanine-ηLys-ηPhe-NH₂
H-ηArg-ηDmt-ηLys-42,6-dimethylphenylalanine-NH₂
H-ηHar-ηDmt-ηLys-η2,6-dimethylphenylalanine-NH₂
H-ηAgb-ηDmt-ηLys-η2,6-dimethylphenylalanine-NH₂
H-ηArg-ηDmt-ηLys-η3-hydroxyphenylalanine-NH₂
H-ηHar-ηDmt-ηLys-η3-hydroxyphenylalanine-NH₂
H-ηAgb-ηDmt-ηLys-η3-hydroxyphenylalanine-NH₂
H-ηArg-ηDmt-ηN6-acetyllysine-ηPhe-NH₂
H-ηHar-ηDmt-ηN6-acetyllysine-ηPhe-NH₂
H-ηAgb-ηDmt-ηN6-acetyllysine-ηPhe-NH₂
H-ηArg-ηPhe-ηLys-ηPhe-NH₂
H-ηHar-ηPhe-ηLys-ηPhe-NH₂
H-ηAgb-ηPhe-ηLys-ηPhe-NH₂
H-ηArg-ηTrp-ηLys-ηPhe-NH₂
H-ηHar-ηTrp-ηLys-ηPhe-NH₂
H-ηAgb-ηTrp-ηLys-ηPhe-NH₂
H-ηArg-ηTyr-ηLys-ηPhe-NH₂
H-ηHar-ηTyr-ηLys-ηPhe-NH₂
H-ηAgb-ηTyr-ηLys-ηPhe-NH₂
H-ηArg-ηDmt-ηLys-η2,6-dimethylphenylalanine-NH₂
H-ηHar-ηDmt-ηLys-η2,6-dimethylphenylalanine-NH₂
H-ηAgb-ηDmt-ηLys-η2,6-dimethylphenylalanine-NH₂
H-ηArg-ηDmt-ηLys-η3-hydroxyphenylalanine-NH₂
H-ηHar-ηDmt-ηLys-η3-hydroxyphenylalanine-NH₂
H-ηAgb-ηDmt-ηLys-η3-hydroxyphenylalanine-NH₂
H-ηArg-ηDmt-ηLys-ηDmt-NH₂
H-ηHar-ηDmt-ηLys-ηDmt-NH₂
H-ηAgb-ηDmt-ηLys-ηDmt-NH₂
H-ηArg-ηDmt-ηLys-ηTrp-NH₂
H-ηHar-ηDmt-ηLys-ηTrp-NH₂
H-ηAgb-ηDmt-ηLys-ηTrp-NH₂
H-ηArg-ηDmt-ηOrn-ηTrp-NH₂
H-ηArg-ηDmt-ηLys-ηTyr-NH₂
H-ηHar-ηDmt-ηLys-ηTyr-NH₂
H-ηAgb-ηDmt-ηLys-ηTyr-NH₂
H-ηArg-ηDmt-ηLys-ηDmt-NH₂
H-ηHar-ηDmt-ηLys-ηDmt-NH₂
H-ηAgb-ηDmt-ηLys-ηDmt-NH₂
H-ηArg-ηDmt-ηLys-ηTrp-NH₂
H-ηHar-ηDmt-ηLys-ηTrp-NH₂
H-ηAgb-ηDmt-ηLys-ηTrp-NH₂
H-ηArg-ηDmt-ηLys-ηTyr-NH₂
H-ηHar-ηDmt-ηLys-ηTyr-NH₂
H-ηAgb-ηDmt-ηLys-ηTyr-NH₂
H-ηArg-ηDmt-ηPhe-ηLys-NH₂
H-ηHar-ηDmt-ηPhe-ηLys-NH₂
H-ηAgb-ηDmt-ηPhe-ηLys-NH₂
H-ηArg-ηDmt-ηN6-acetyllysine-ηPhe-NH₂
H-ηHar-ηDmt-ηN6-acetyllysine-ηPhe-NH₂
H-ηAgb-ηDmt-ηN6-acetyllysine-ηPhe-NH₂
H-ηArg-ηLys-ηDmt-ηPhe-NH₂
H-ηHar-ηLys-ηDmt-ηPhe-NH₂
H-ηAgb-ηLys-ηDmt-ηPhe-NH₂
H-ηArg-ηLys-ηPhe-ηDmt-NH₂
H-ηHar-ηLys-ηPhe-ηDmt-NH₂
H-ηAgb-ηLys-ηPhe-ηDmt-NH₂
H-ηArg-ηPhe-ηDmt-ηLys-NH₂
H-ηHar-ηPhe-ηDmt-ηLys-NH₂
H-ηAgb-ηPhe-ηDmt-ηLys-NH₂
H-ηArg-ηPhe-ηLys-ηDmt-NH₂
H-ηHar-ηPhe-ηLys-ηDmt-NH₂
H-ηAgb-ηPhe-ηLys-ηDmt-NH₂
H-ηArg-ηPhe-ηLys-ηPhe-NH₂
H-ηHar-ηPhe-ηLys-ηPhe-NH₂
H-ηAgb-ηPhe-ηLys-ηPhe-NH₂
H-ηArg-ηTrp-ηLys-ηPhe-NH₂
H-ηHar-ηTrp-ηLys-ηPhe-NH₂
H-ηAgb-ηTrp-ηLys-ηPhe-NH₂
H-ηArg-ηTyr-ηLys-ηPhe-NH₂
H-ηHar-ηTyr-ηLys-ηPhe-NH₂
H-ηAgb-ηTyr-ηLys-ηPhe-NH₂
H-ηArg-ηPhe-ηLys-ηDmt-NH₂
H-ηHar-ηPhe-ηLys-ηDmt-NH₂
H-ηAgb-ηPhe-ηLys-ηDmt-NH₂
H-ηArg-ηTyr-ηLys-ηPhe-NH₂
H-ηHar-ηTyr-ηLys-ηPhe-NH₂
H-ηAgb-ηTyr-ηLys-ηPhe-NH₂
H-ηHis-ηDmt-ηLys-ηPhe-NH₂
H-ηLys-ηDmt-ηLys-ηPhe-NH₂
H-ηOrn-ηDmt-ηOrn-ηPhe-NH₂
H-ηDmt-ηArg-ηLys-ηPhe-NH₂
H-ηDmt-ηHar-ηLys-ηPhe-NH₂
H-ηDmt-ηAgb-ηLys-ηPhe-NH₂
H-ηDmt-ηArg-ηPhe-ηLys-NH₂
H-ηDmt-ηHar-ηPhe-ηLys-NH₂
H-ηDmt-ηAgb-ηPhe-ηLys-NH₂
H-ηDmt-ηLys-ηArg-ηPhe-NH₂
H-ηDmt-ηLys-ηHar-ηPhe-NH₂
H-ηDmt-ηLys-ηAgb-ηPhe-NH₂
H-ηDmt-ηLys-ηPhe-ηArg-NH₂
H-ηDmt-ηLys-ηPhe-ηHar-NH₂
H-ηDmt-ηLys-ηPhe-ηAgb-NH₂
H-ηDmt-ηPhe-ηArg-ηLys-NH₂
H-ηDmt-ηPhe-ηHar-ηLys-NH₂
H-ηDmt-ηPhe-ηAgb-ηLys-NH₂
H-ηDmt-ηPhe-ηLys-ηArg-NH₂
H-ηDmt-ηPhe-ηLys-ηHar-NH₂
H-ηDmt-ηPhe-ηLys-ηAgb-NH₂
H-ηDmt-ηArg-ηLys-ηPhe-NH₂
H-ηDmt-ηHar-ηLys-ηPhe-NH₂
H-ηDmt-ηAgb-ηLys-ηPhe-NH₂
H-ηDmt-ηArg-ηPhe-ηLys-NH₂
H-ηDmt-ηHar-ηPhe-ηLys-NH₂
H-ηDmt-ηAgb-ηPhe-ηLys-NH₂
H-ηDmt-ηLys-ηArg-ηPhe-NH₂
H-ηDmt-ηLys-ηHar-ηPhe-NH₂
H-ηDmt-ηLys-ηAgb-ηPhe-NH₂
H-ηDmt-ηLys-ηPhe-ηArg-NH₂
H-ηDmt-ηLys-ηPhe-ηHar-NH₂
H-ηDmt-ηLys-ηPhe-ηAgb-NH₂
H-ηDmt-ηPhe-ηArg-ηLys-NH₂
H-ηDmt-ηPhe-ηHar-ηLys-NH₂
H-ηDmt-ηPhe-ηAgb-ηLys-NH₂
H-ηDmt-ηPhe-ηLys-ηArg-NH₂
H-ηDmt-ηPhe-ηLys-ηHar-NH₂
H-ηDmt-ηPhe-ηLys-ηAgb-NH₂
H-ηHis-ηDmt-ηLys-ηPhe-NH₂
H-ηLys-ηArg-ηDmt-ηPhe-NH₂
H-ηLys-ηHar-ηDmt-ηPhe-NH₂
H-ηLys-ηAgb-ηDmt-ηPhe-NH₂
H-ηLys-ηArg-ηPhe-ηDmt-NH₂
H-ηLys-ηHar-ηPhe-ηDmt-NH₂
H-ηLys-ηAgb-ηPhe-ηDmt-NH₂
H-ηLys-ηDmt-ηArg-ηPhe-NH₂
H-ηLys-ηDmt-ηHar-ηPhe-NH₂
H-ηLys-ηDmt-ηAgb-ηPhe-NH₂
H-ηLys-ηDmt-ηLys-ηPhe-NH₂
H-ηLys-ηDmt-ηPhe-ηArg-NH₂
H-ηLys-ηDmt-ηPhe-ηHar-NH₂
H-ηLys-ηDmt-ηPhe-ηAgb-NH₂
H-ηLys-ηPhe-ηArg-ηDmt-NH₂
H-ηLys-ηPhe-ηHar-ηDmt-NH₂
H-ηLys-ηPhe-ηAgb-ηDmt-NH₂
H-ηLys-ηPhe-ηDmt-ηArg-NH₂
H-ηLys-ηPhe-ηDmt-ηHar-NH₂
H-ηLys-ηPhe-ηDmt-ηAgb-NH₂
H-ηPhe-ηArg-ηDmt-ηLys-NH₂
H-ηPhe-ηHar-ηDmt-ηLys-NH₂
H-ηPhe-ηAgb-ηDmt-ηLys-NH₂
H-ηPhe-ηArg-ηLys-ηDmt-NH₂
H-ηPhe-ηHar-ηLys-ηDmt-NH₂
H-ηPhe-ηAgb-ηLys-ηDmt-NH₂
H-ηPhe-ηDmt-ηArg-ηLys-NH₂
H-ηPhe-ηDmt-ηHar-ηLys-NH₂
H-ηPhe-ηDmt-ηAgb-ηLys-NH₂
H-ηPhe-ηDmt-ηLys-ηArg-NH₂
H-ηPhe-ηDmt-ηLys-ηHar-NH₂
H-ηPhe-ηDmt-ηLys-ηAgb-NH₂
H-ηPhe-ηLys-ηArg-ηDmt-NH₂
H-ηPhe-ηLys-ηHar-ηDmt-NH₂
H-ηPhe-ηLys-ηAgb-ηDmt-NH₂
H-ηPhe-ηLys-ηDmt-ηArg-NH₂
H-ηPhe-ηLys-ηDmt-ηHar-NH₂
H-ηPhe-ηLys-ηDmt-ηAgb-NH₂
H-ηLys-ηArg-ηDmt-ηPhe-NH₂
H-ηLys-ηHar-ηDmt-ηPhe-NH₂
H-ηLys-ηAgb-ηDmt-ηPhe-NH₂
H-ηLys-ηArg-ηPhe-ηDmt-NH₂
H-ηLys-ηHar-ηPhe-ηDmt-NH₂
H-ηLys-ηAgb-ηPhe-ηDmt-NH₂
H-ηLys-ηDmt-ηArg-ηPhe-NH₂
H-ηLys-ηDmt-ηHar-ηPhe-NH₂
H-ηLys-ηDmt-ηAgb-ηPhe-NH₂
H-ηLys-ηDmt-ηPhe-ηArg-NH₂
H-ηLys-ηDmt-ηPhe-ηHar-NH₂
H-ηLys-ηDmt-ηPhe-ηAgb-NH₂
H-ηLys-ηPhe-ηArg-ηDmt-NH₂
H-ηLys-ηPhe-ηHar-ηDmt-NH₂
H-ηLys-ηPhe-ηAgb-ηDmt-NH₂
H-ηLys-ηPhe-ηDmt-ηArg-NH₂
H-ηLys-ηPhe-ηDmt-ηHar-NH₂
H-ηLys-ηPhe-ηDmt-ηAgb-NH₂
H-ηPhe-ηArg-ηPhe-ηLys-NH₂
H-ηPhe-ηHar-ηPhe-ηLys-NH₂
H-ηPhe-ηAgb-ηPhe-ηLys-NH₂
H-ηPhe-ηArg-ηDmt-ηLys-NH₂
H-ηPhe-ηHar-ηDmt-ηLys-NH₂
H-ηPhe-ηAgb-ηDmt-ηLys-NH₂
H-ηPhe-ηArg-ηLys-ηDmt-NH₂
H-ηPhe-ηHar-ηLys-ηDmt-NH₂
H-ηPhe-ηAgb-ηLys-ηDmt-NH₂
H-ηPhe-ηDmt-ηArg-ηLys-NH₂
H-ηPhe-ηDmt-ηHar-ηLys-NH₂
H-ηPhe-ηDmt-ηAgb-ηLys-NH₂
H-ηPhe-ηDmt-ηLys-ηArg-NH₂
H-ηPhe-ηDmt-ηLys-ηHar-NH₂
H-ηPhe-ηDmt-ηLys-ηAgb-NH₂
H-ηPhe-ηLys-ηArg-ηDmt-NH₂
H-ηPhe-ηLys-ηHar-ηDmt-NH₂
H-ηPhe-ηLys-ηAgb-ηDmt-NH₂
H-ηPhe-ηLys-ηDmt-ηArg-NH₂
H-ηPhe-ηLys-ηDmt-ηHar-NH₂
H-ηPhe-ηLys-ηDmt-ηAgb-NH₂
ηArg-ηDmt-ηLys-ηPhe-NH₂
ηHar-ηDmt-ηLys-ηPhe-NH₂
ηAgb-ηDmt-ηLys-ηPhe-NH₂
ηLys-ηDmt-ηArg
ηLys-ηDmt-ηHar
ηLys-ηDmt-ηAgb
ηLys-ηPhe
ηLys-ηPhe-ηArg-ηDmt
ηLys-ηPhe-ηHar-ηDmt
ηLys-ηPhe-ηAgb-ηDmt
ηLys-ηTrp-ηArg
ηLys-ηTrp-ηHar
ηLys-ηTrp-ηAgb
ηPhe-ηArg-ηDmt-ηLys
ηPhe-ηHar-ηDmt-ηLys
ηPhe-ηAgb-ηDmt-ηLys
ηPhe-ηArg-ηPhe-ηLys
ηPhe-ηHar-ηPhe-ηLys
ηPhe-ηAgb-ηPhe-ηLys
ηPhe-ηDmt-ηArg-ηLys
ηPhe-ηDmt-ηHar-ηLys
ηPhe-ηDmt-ηAgb-ηLys
ηPhe-ηLys-ηDmt
ηArg-ηDmt-ηLys-ηPhe-NH₂
ηHar-ηDmt-ηLys-ηPhe-NH₂
ηAgb-ηDmt-ηLys-ηPhe-NH₂
ηPhe-ηDmt-ηArg-ηLys-NH₂
ηPhe-ηDmt-ηHar-ηLys-NH₂
ηPhe-ηDmt-ηAgb-ηLys-NH₂
ηPhe-ηLys-ηDmt-ηArg-NH₂
ηPhe-ηLys-ηDmt-ηHar-NH₂
ηPhe-ηLys-ηDmt-ηAgb-NH₂
ηDmt-ηArg-ηLys-ηPhe-NH₂
ηDmt-ηHar-ηLys-ηPhe-NH₂
ηDmt-ηAgb-ηLys-ηPhe-NH₂
ηLys-ηDmt-ηArg-ηPhe-NH₂
ηLys-ηDmt-ηHar-ηPhe-NH₂
ηLys-ηDmt-ηAgb-ηPhe-NH₂
ηPhe-ηDmt-ηLys-ηArg-NH₂
ηPhe-ηDmt-ηLys-ηHar-NH₂
ηPhe-ηDmt-ηLys-ηAgb-NH₂
ηArg-ηLys-ηDmt-ηPhe-NH₂
ηHar-ηLys-ηDmt-ηPhe-NH₂
ηAgb-ηLys-ηDmt-ηPhe-NH₂
ηArg-ηDmt-ηPhe-ηLys-NH₂
ηHar-ηDmt-ηPhe-ηLys-NH₂
ηAgb-ηDmt-ηPhe-ηLys-NH₂
ηArg-ηDmt-ηLys-ηPhe-NH₂
ηHar-ηDmt-ηLys-ηPhe-NH₂
ηAgb-ηDmt-ηLys-ηPhe-NH₂
ηDmt-ηArg-ηPhe-ηLys-NH₂
ηDmt-ηHar-ηPhe-ηLys-NH₂
ηDmt-ηAgb-ηPhe-ηLys-NH₂
H-ηPhe-ηArg-ηPhe-ηLys-ηCys-NH₂
H-ηPhe-ηHar-ηPhe-ηLys-ηCys-NH₂
H-ηPhe-ηAgb-ηPhe-ηLys-ηCys-NH₂
ηArg-ηDmt-ηLys-ηTrp-NH₂
ηHar-ηDmt-ηLys-ηTrp-NH₂
ηAgb-ηDmt-ηLys-ηTrp-NH₂
ηArg-ηTrp-ηLys-ηTrp-NH₂
ηHar-ηTrp-ηLys-ηTrp-NH₂
ηAgb-ηTrp-ηLys-ηTrp-NH₂
ηArg-η2′6′Dmt-ηLys-ηPhe-NH₂
ηHar-η2′6′Dmt-ηLys-ηPhe-NH₂
ηAgb-η2′6′Dmt-ηLys-ηPhe-NH₂
ηArg-η2′6′Dmt-ηOrn-ηTrp-NH₂
H-ηPhe-ηArg-ηPhe-ηLys-ηCys-NH₂
H-ηPhe-ηHar-ηPhe-ηLys-ηCys-NH₂
H-ηPhe-ηAgb-ηPhe-ηLys-ηCys-NH₂
ηArg-ηDmt-ηLys-ηPhe-ηSer-ηCys-NH₂
ηHar-ηDmt-ηLys-ηPhe-ηSer-ηCys-NH₂
ηAgb-ηDmt-ηLys-ηPhe-ηSer-ηCys-NH₂
ηArg-ηDmt-ηLys-ηPhe-Gly-ηCys-NH₂
ηHar-ηDmt-ηLys-ηPhe-Gly-ηCys-NH₂
ηAgb-ηDmt-ηLys-ηPhe-Gly-ηCys-NH₂
Gly-ηPhe-ηLys-ηHis-ηArg-ηTyr-NH₂
Gly-ηPhe-ηLys-ηHis-ηHar-ηTyr-NH₂
Gly-ηPhe-ηLys-ηHis-ηAgb-ηTyr-NH₂
ηArg-ηDmt-ηLys-ηPhe-ηMet-NH₂
ηHar-ηDmt-ηLys-ηPhe-ηMet-NH₂
ηAgb-ηDmt-ηLys-ηPhe-ηMet-NH₂
ηArg-ηDmt-ηLys-ηPhe-ηLys-ηTrp-NH₂
ηHar-ηDmt-ηLys-ηPhe-ηLys-ηTrp-NH₂
ηAgb-ηDmt-ηLys-ηPhe-ηLys-ηTrp-NH₂
ηArg-ηDmt-ηLys-ηDmt-ηLys-ηTrp-NH₂
ηHar-ηDmt-ηLys-ηDmt-ηLys-ηTrp-NH₂
ηAgb-ηDmt-ηLys-ηDmt-ηLys-ηTrp-NH₂
ηArg-ηDmt-ηLys-ηPhe-ηLys-ηMet-NH₂
ηHar-ηDmt-ηLys-ηPhe-ηLys-ηMet-NH₂
ηAgb-ηDmt-ηLys-ηPhe-ηLys-ηMet-NH₂
ηArg-ηDmt-ηLys-ηDmt-ηLys-ηMet-NH₂
ηHar-ηDmt-ηLys-ηDmt-ηLys-ηMet-NH₂
ηAgb-ηDmt-ηLys-ηDmt-ηLys-ηMet-NH₂
H-ηArg-ηDmt-ηLys-OH
H-ηHar-ηDmt-ηLys-OH
H-ηAgb-ηDmt-ηLys-OH
H-ηArg-ηDmt-OH
H-ηHar-ηDmt-OH
H-ηAgb-ηDmt-OH
H-ηArg-ηDmt-ηLys-ηPhe-OH
H-ηHar-ηDmt-ηLys-ηPhe-OH
H-ηAgb-ηDmt-ηLys-ηPhe-OH
Homoarginine (Har)
2-amino-4-guandinyl butyric acid (Agb)
2′-methyltyrosine (Mmt)
Dimethyltyrosine (Dmt)
2′,6′ -dimethyltyrosine (2′6′-Dmt)
3′,5′-dimethyηtyrosine (3′5′Dmt)
2′-hydroxy-6′-methyltyrosine (Hmt)
2′-methylphenylalanine (Mmp)
dimethylphenylalanine (Dmp)
2′,6′-dimethylphenylalanine (2′,6′-Dmp)
2′-hydroxy-6′-methylphenylalanine (Hmp)
cyclohexylalanine (Cha)
diaminobutyric (Dab)
diaminopropionic acid (Dap)
β-dansyl-L-α,β-diaminopropionic acid (dnsDap)
β-anthraniloyl-L-α,β-diaminopropionic acid (atnDap)
biotin (bio)
norleucine (Nle)
2-aminohepantoic acid (Ahp)
β-(6′-dimethylamino-2′-naphthoyl)alanine (Aid)
Sarcosine (Sar)

TABLE B

| Monomer | Monomer | Monomer | Monomer | C-Terminal |
|---|---|---|---|---|
| Position 1 | Position 2 | Position 3 | Position 4 | Modification |
| ηTyr | ηArg | ηPhe | ηOrn | NH₂ |
| ηTyr | ηArg | ηPhe | ηDab | NH₂ |
| ηTyr | ηArg | ηPhe | ηDap | NH₂ |
| η2′6′Dmt | ηArg | ηPhe | ηLys- | NH₂ |
| | | | NH(CH₂)₂-NH- | |
| | | | dnsDap | |
| η2′6′Dmt | ηArg | ηPhe | ηLys- | NH₂ |
| | | | NH(CH₂)₂-NH- | |
| | | | atnDap | |
| η2′6′Dmt | ηArg | ηPhe | ηdnsLys | NH₂ |
| η2′6′Dmt | ηcit | ηPhe | ηAhp | NH₂ |
| η2′6′Dmt | ηArg | ηPhe | ηDab | NH₂ |
| η2′6′Dmt | ηArg | ηPhe | ηDap | NH₂ |
| η2′6′Dmt | ηArg | ηPhe | ηLys | NH₂ |
| η2′6′Dmt | ηArg | ηPhe | ηOrn | NH₂ |
| η2′6′Dmt | ηArg | ηPhe | ηDab | NH₂ |
| η2′6′Dmt | ηArg | ηPhe | ηDap | NH₂ |
| ηTyr | ηArg | ηTyr | ηLys | NH₂ |
| ηTyr | ηArg | ηTyr | ηOrn | NH₂ |
| ηTyr | ηArg | ηTyr | ηDab | NH₂ |
| ηTyr | ηArg | ηTyr | ηDap | NH₂ |
| η2′6′Dmt | ηArg | ηTyr | ηLys | NH₂ |
| η2′6′Dmt | ηArg | ηTyr | ηOrn | NH₂ |
| η2′6′Dmt | ηArg | ηTyr | ηDab | NH₂ |
| η2′6′Dmt | ηArg | ηTyr | ηDap | NH₂ |
| η2′6′Dmt | ηArg | η2′6′Dmt | ηLys | NH₂ |
| η2′6′Dmt | ηArg | η2′6′Dmt | ηOrn | NH₂ |
| η2′6′Dmt | ηArg | η2′6′Dmt | ηDab | NH₂ |
| η2′6′Dmt | ηArg | η2′6′Dmt | ηDap | NH₂ |
| η3′5′Dmt | ηArg | η3′5′Dmt | ηArg | NH₂ |
| η3′5′Dmt | ηArg | η3′5′Dmt | ηLys | NH₂ |
| η3′5′Dmt | ηArg | η3′5′Dmt | ηOrn | NH₂ |
| η3′5′Dmt | ηArg | η3′5′Dmt | ηDab | NH₂ |
| ηTyr | ηLys | ηPhe | ηDap | NH₂ |
| ηTyr | ηLys | ηPhe | ηArg | NH₂ |
| ηTyr | ηLys | ηPhe | ηLys | NH₂ |
| ηTyr | ηLys | ηPhe | ηOrn | NH₂ |
| η2′6′Dmt | ηLys | ηPhe | ηDab | NH₂ |
| η2′6′Dmt | ηLys | ηPhe | ηDap | NW |
| η2′6′Dmt | ηLys | ηPhe | ηArg | NH₂ |
| η2′6′Dmt | ηLys | ηPhe | ηLys | NH₂ |
| η3′5′Dmt | ηLys | ηPhe | ηOrn | NH₂ |
| η3′5′Dmt | ηLys | ηPhe | ηDab | NH₂ |
| η3′5′Dmt | ηLys | ηPhe | ηDap | NH₂ |
| η3′5′Dmt | ηLys | ηPhe | ηArg | NH₂ |
| ηTyr | ηLys | ηTyr | ηLys | NH₂ |
| ηTyr | ηLys | ηTyr | ηOrn | NH₂ |
| ηTyr | ηLys | ηTyr | ηDab | NH₂ |
| ηTyr | ηLys | ηTyr | ηDap | NH₂ |
| η2′6′Dmt | ηLys | ηTyr | ηLys | NH₂ |
| η2′6′Dmt | ηLys | ηTyr | ηOrn | NH₂ |
| η2′6′Dmt | ηLys | ηTyr | ηDab | NH₂ |
| η2′6′Dmt | ηLys | ηTyr | ηDap | NH₂ |
| η2′6′Dmt | ηLys | η2′6′Dmt | ηLys | NH₂ |
| η2′6′Dmt | ηLys | η2′6′Dmt | ηOrn | NH₂ |
| η2′6′Dmt | ηLys | η2′6′Dmt | ηDab | NH₂ |
| η2′6′Dmt | ηLys | η2′6′Dmt | ηDap | NH₂ |
| η3′5′Dmt | ηLys | η3′5′Dmt | ηLys | NH₂ |
| η3′5′Dmt | ηLys | η3′5′Dmt | ηOrn | NH₂ |
| η3′5′Dmt | ηLys | η3′5′Dmt | ηDab | NH₂ |
| η3′5′Dmt | ηLys | η3′5′Dmt | ηDap | NH₂ |
| ηTyr | ηLys | ηPhe | ηArg | NH₂ |
| ηTyr | ηOrn | ηPhe | ηArg | NH₂ |
| ηTyr | ηDab | ηPhe | ηArg | NH₂ |
| ηTyr | ηDap | ηPhe | ηArg | NH₂ |
| η2′6′Dmt | ηArg | ηPhe | ηArg | NH₂ |
| η2′6′Dmt | ηLys | ηPhe | ηArg | NH₂ |
| η2′6′Dmt | ηOrn | ηPhe | ηArg | NH₂ |
| η2′6′Dmt | ηDab | ηPhe | ηArg | NH₂ |
| η3′5′Dmt | ηDap | ηPhe | ηArg | NH₂ |
| η3′5′Dmt | ηArg | ηPhe | ηArg | NH₂ |
| η3′5′Dmt | ηLys | ηPhe | ηArg | NH₂ |
| η3′5′Dmt | ηOrn | ηPhe | ηArg | NH₂ |
| ηTyr | ηLys | ηTyr | ηArg | NH₂ |
| ηTyr | ηOrn | ηTyr | ηArg | NH₂ |
| ηTyr | ηDab | ηTyr | ηArg | NH₂ |
| ηTyr | ηDap | ηTyr | ηArg | NH₂ |
| η2′6′Dmt | ηArg | η2′6′Dmt | ηArg | NH₂ |
| η2′6′Dmt | ηLys | η2′6′Dmt | ηArg | NH₂ |
| η2′6′Dmt | ηOrn | η2′6′Dmt | ηArg | NH₂ |
| η2′6′Dmt | ηDab | η2′6′Dmt | ηArg | NH₂ |
| η3′5′Dmt | ηDap | η3′5′Dmt | ηArg | NH₂ |
| η3′5′Dmt | ηArg | η3′5′Dmt | ηArg | NH₂ |
| η3′5′Dmt | ηLys | η3′5′Dmt | ηArg | NH₂ |
| η3′5′Dmt | ηOrn | η3′5′Dmt | ηArg | NH₂ |
| ηMmt | ηArg | ηPhe | ηLys | NH₂ |
| ηMmt | ηArg | ηPhe | ηOrn | NH₂ |
| ηMmt | ηArg | ηPhe | ηDab | NH₂ |
| ηMmt | ηArg | ηPhe | ηDap | NH₂ |
| ηHmt | ηArg | ηPhe | ηLys | NH₂ |
| ηHmt | ηArg | ηPhe | ηOrn | NH₂ |
| ηHmt | ηArg | ηPhe | ηDab | NH₂ |
| ηHmt | ηArg | ηPhe | ηDap | NH₂ |
| ηMmt | ηLys | ηPhe | ηLys | NH₂ |
| ηMmt | ηLys | ηPhe | ηOrn | NH₂ |
| ηMmt | ηLys | ηPhe | ηDab | NH₂ |
| ηMmt | ηLys | ηPhe | ηDap | NH₂ |
| ηMmt | ηLys | ηPhe | ηArg | NH₂ |
| ηHmt | ηLys | ηPhe | ηLys | NH₂ |
| ηHmt | ηLys | ηPhe | ηOrn | NH₂ |
| ηHmt | ηLys | ηPhe | ηDab | NH₂ |
| ηHmt | ηLys | ηPhe | ηDap | NH₂ |
| ηHmt | ηLys | ηPhe | ηArg | NH₂ |
| ηMmt | ηLys | ηPhe | ηArg | NH₂ |
| ηMmt | ηOrn | ηPhe | ηArg | NH₂ |
| ηMmt | ηDab | ηPhe | ηArg | NH₂ |
| ηMmt | ηDap | ηPhe | ηArg | NH₂ |
| ηMmt | ηArg | ηPhe | ηArg | NH₂ |
| ηHmt | ηLys | ηPhe | ηArg | NH₂ |
| ηHmt | ηOrn | ηPhe | ηArg | NH₂ |
| ηHmt | ηDab | ηPhe | ηArg | NH₂ |
| ηHmt | ηDap | ηPhe | ηArg | NH₂ |
| ηHmt | ηArg | ηPhe | ηArg | NH₂ |
| ηTrp | ηArg | ηPhe | ηLys | NH₂ |

2′-methyltyrosine (Mmt)
Dimethyltyrosine (Dmt)
2′,6′-dimethyltyrosine (2′6′-Dmt)
3′,5′-dimethyltyrosine (3′5′-Dmt)
2′-hydroxy-6′-methyltyrosine (Hmt)
2′-methylphenylalanine (Mmp)
dimethylphenylalanine (Dmp)
2′,6′-dimethylphenylalanine (2′,6′-Dmp)
2′-hydroxy-6′-methylphenylalanine (Hmp)
cyclohexyl alanine (Cha)
diaminobutyric (Dab)
diaminopropionic acid (Dap)
β-dansyl-L-α,β-diaminopropionic acid (dnsDap)
β-anthraniloyl-L-a,β-diaminopropionic acid (atnDap)
β-dansyl-lysine (dnsLys)
biotin (bio)
norleucine (Nle)
2-aminohepantoic acid (Ahp)
β-(6′-dimethylamino-2′-naphthoyl)alanine (Ald)
Sarcosine (Sar)

Peptoids of the present technology include those where ηhomoarginine (ηHar) or η2-amino-4-guandinyl butyric acid (ηAgb) are used where ηArg is indicated in Table B.

In some embodiments, the peptoid is defined by Formula II:

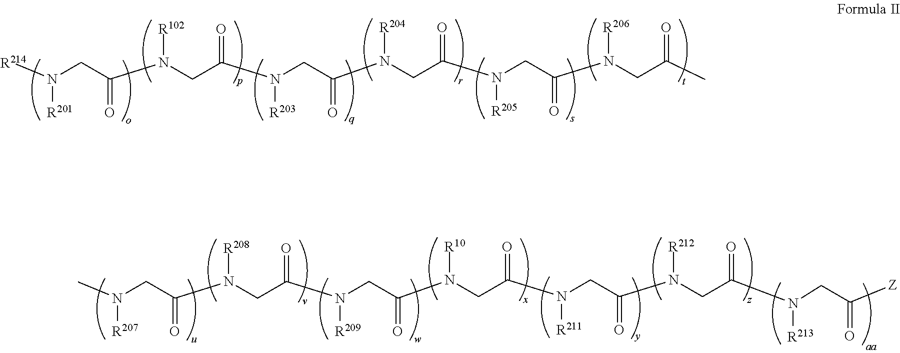

wherein in Formula II:
Z is —N(R²¹⁶)(R²¹⁷) or —O—R²¹⁸;
R²⁰¹ is

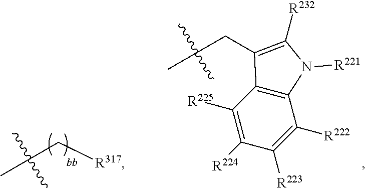

or R²¹⁵;
R²⁰² is

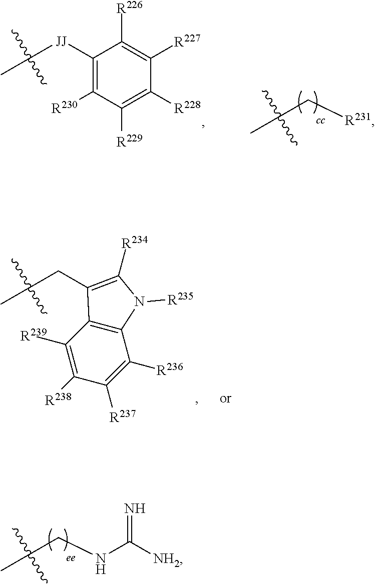

or optionally R²¹⁵ if o is 0;
R²⁰³ is

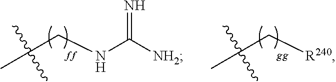

or hydrogen, or optionally R²¹⁵ if o and p are each 0;
R²⁰⁴ is

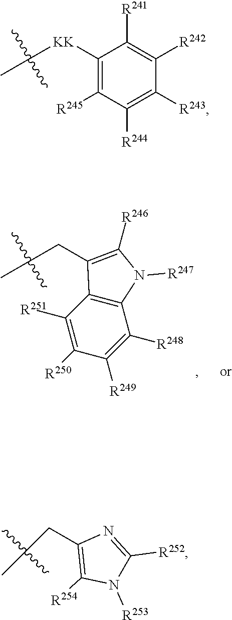

or optionally R²¹⁵ if o, p, and q are each 0;
R²⁰⁵ is

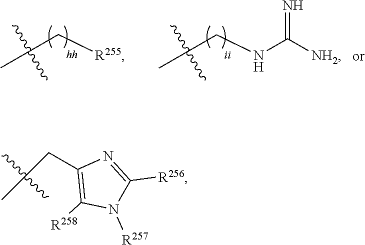

or optionally R²¹⁵ if o, p, q, and r are each 0;

R²⁰⁶ is

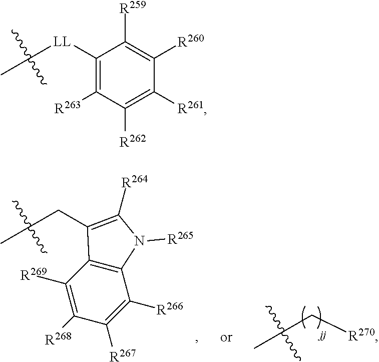

or optionally R²¹⁵ if o, p, q, r, and s are each 0;

R²⁰⁷ is

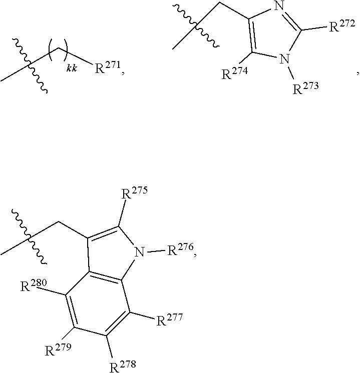

or hydrogen, or optionally R²¹⁵ if o, p, q, r, s, and t are each 0;
R²⁰⁸ is

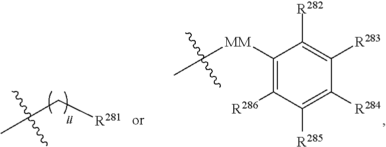

or optionally R²¹⁵ if o, p, q, r, s, t, and u are each 0;
R²⁰⁹ is

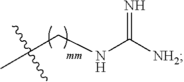

R²¹⁰ is

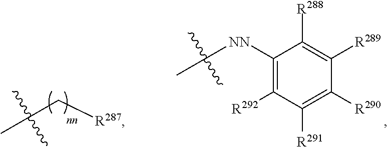

or hydrogen;
R²¹¹ is

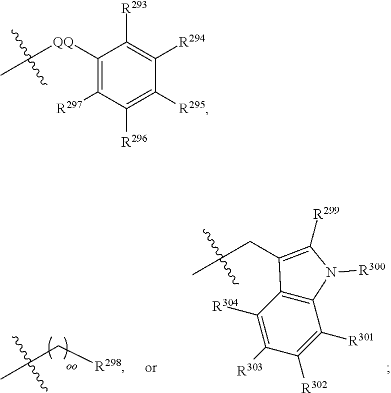

R²¹² is

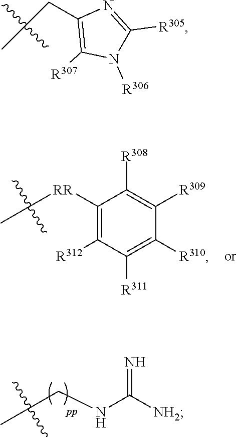

R²¹³ is

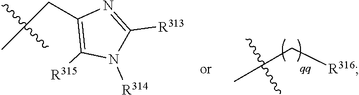

wherein
   R²¹⁴, R²¹⁵, R²¹⁶, R²¹⁷, and R²¹⁸ are each independently a hydrogen or substituted or unsubstituted C₁-C₆ alkyl, C₂-C₆ alkenyl, C₂-C₆ alkynyl, saturated or unsaturated cycloalkyl, cycloalkylalkyl, aryl, aralkyl, 5- or 6-membered saturated or unsaturated heterocylyl, heteroaryl, or amino protecting group; or R²¹⁴ and R²¹⁵ together or R²¹⁶ and R²¹⁷ together form a 3, 4, 5, 6, 7, or 8 membered substituted or unsubstituted heterocycyl ring;
   R²²², R²²³, R²²⁴, R²²⁵, R²²⁶, R²²⁷, R²²⁸, R²²⁹, R²³⁰, R²³², R²³⁴, R²³⁶, R²³⁷, R²³⁸, R²³⁹, R²⁴¹, R²⁴², R²⁴³, R²⁴⁴, R²⁴⁵, R²⁴⁶, R²⁴⁸, R²⁴⁹, R²⁵⁰, R²⁵¹, R²⁵², R²⁵⁴, R²⁵⁶, R²⁵⁸, R²⁵⁹, R²⁶⁰, R²⁶¹, R²⁶², R²⁶³, R²⁶⁴, R²⁶⁶, R²⁶⁷, R²⁶⁸, R²⁶⁹, R²⁷², R²⁷⁴, R²⁷⁵, R²⁷⁷, R²⁷⁸, R²⁷⁹, R²⁸⁰, R²⁸², R²⁸³, R²⁸⁴, R²⁸⁵, R²⁸⁶, R²⁸⁸, R²⁸⁹, R²⁹⁰, R²⁹¹, R²⁹², R²⁹³, R²⁹⁴, R²⁹⁵, R²⁹⁶, R²⁹⁷, R²⁹⁹, R³⁰¹, R³⁰², R³⁰³, R³⁰⁴, R³⁰⁵, R³⁰⁷, R³⁰⁸, R³⁰⁹, R³¹⁰, R³¹¹, R³¹², R³¹³, and R³¹⁵ are each independently a hydrogen, amino, amido, —NO₂, —CN, —OR^{c}, SR^{c}, —NR^{c}R^{c}, —F, —Cl, —Br, —I, or a substituted or unsubstituted C₁-C₆ alkyl, C₁-C₆ alkoxy, —C(O)-alkyl, —C(O)-aryl, —C(O)-aralkyl, —C(O)₂R^{c}, C₁-C₄ alkylamino, C₁-C₄ dialkylamino, or perhaloalkyl group;
   R²²¹, R²³⁵, R²⁴⁷, R²⁵³, R²⁵⁷, R²⁶⁵, R²⁷³, R²⁷⁶, R³⁰⁰, R³⁰⁶, and R³¹⁴ are each independently a hydrogen or substituted or unsubstituted C₁-C₆ alkyl group;
   R²³¹, R²⁴⁰, R²⁵⁵, R²⁷⁰, R²⁷¹, R²⁸¹, R²⁸⁷, R²⁹⁸, R³¹⁶, and R³¹⁷ are each independently a hydrogen, —OR^{c}, —SR^{c}, —NR^{c}R^{c}, —NR^{c}R^{d}, —CO₂R^{c}, —(CO)NR^{c}R^{c}, —NR^{c}(CO)R^{c}, —NR^{c}C(NH)NH₂, —NR^{c}-dansyl, enamine, imine, or a substituted or unsubstituted alkyl, heterocyclyl, aryl, heteroaryl, or aralkyl group;
   JJ, KK, LL, MM, NN, QQ, and RR are each independently absent, —NH(CO)—, or —CH₂—;
   R^{c} at each occurrence is independently a hydrogen or a substituted or unsubstituted C₁-C₆ alkyl group;
   R^{d} at each occurrence is independently a C₁-C₆ alkylene-NR^{c}-dansyl or C₁-C₆ alkylene-NR^{c}-anthraniloyl group;
   o, p, q, r, s, t, u, v, w, x, y, z, and aa are each independently 0 or 1,
      with the proviso that o+p+q+r+s+t+u+v+w+x+y+z+aa equals 6, 7, 8, 9, 10, or 11; and
   bb, cc, ee, ff gg, hh, ii, jj, kk, ii, mm, nn, oo, pp, and qq are each independently 1, 2, 3, 4, or 5.

In any embodiment herein of a peptoid of Formula II, it may be that
R²¹⁴, R²¹⁵, R²¹⁶, R²¹⁷, and R²¹⁸ are each independently a hydrogen or substituted or unsubstituted C₁-C₆ alkyl group;
R²²², R²²³, R²²⁴, R²²⁵, R²²⁶, R²²⁷, R²²⁸, R²²⁹, R²³⁰, R²³², R²³⁴, R²³⁶, R²³⁷, R²³⁸, R²³⁹, R²⁴¹, R²⁴², R²⁴³, R²⁴⁴, R²⁴⁵, R²⁴⁶, R²⁴⁸, R²⁴⁹, R²⁵⁰, R²⁵¹, R²⁵², R²⁵⁴, R²⁵⁶, R²⁵⁸, R²⁵⁹, R²⁶⁰, R²⁶¹, R²⁶², R²⁶³, R²⁶⁴, R²⁶⁶, R²⁶⁷, R²⁶⁸, R²⁶⁹, R²⁷², R²⁷⁴, R²⁷⁵, R²⁷⁷, R²⁷⁸, R²⁷⁹, R²⁸⁰, R²⁸², R²⁸³, R²⁸⁴, R²⁸⁵, R²⁸⁶, R²⁸⁸, R²⁸⁹, R²⁹⁰, R²⁹¹, R²⁹², R²⁹³, R²⁹⁴, R²⁹⁵, R²⁹⁶, R²⁹⁷, R²⁹⁹, R³⁰¹, R³⁰², R³⁰³, R³⁰⁴, R³⁰⁵, R³⁰⁷, R³⁰⁸, R³⁰⁹, R³¹⁰, R³¹¹, R³¹², R³¹³, and R³¹⁵ are each independently a hydrogen, methyl, or —OR^{c} group;
R²³¹ is —(CO)NR^{c}R^{c}, —OR^{c}, or a C₁-C₆ alkyl group, optionally substituted with a hydroxyl or methyl group;
R²⁴⁰ and R²⁵⁵ are each independently —CO₂R^{c} or —NR^{c}R^{c};
R²⁷⁰ and R²⁷¹ are each independently —CO₂R^{c};
R²⁸¹ is —SR^{c} or —NR^{c}R^{c};
R²⁸⁷—(CO)NR^{c}R^{c} or —OR^{c};
R²⁹⁸—NR^{c}R^{c}, —CO₂R^{c}, or —SR^{c};
R³¹⁶ is —NR^{c}R^{c};
R³¹⁷ is hydrogen or —NR^{c}R^{c}; and
JJ, KK, LL, MM, NN, QQ, and RR are each independently absent or —CH₂—.

In any embodiment herein of a peptoid of Formula II, it may be that
R²²¹, R²²², R²²³, R²²⁴, R²²⁵, R²²⁶, R²²⁷, R²²⁸, R²²⁹, R²³⁰, R²³², R²³⁴, R²³⁵, R²³⁶, R²³⁷, R²³⁸, R²³⁹, R²⁴², R²⁴⁴, R²⁴⁶, R²⁴⁷, R²⁴⁸, R²⁴⁹, R²⁵⁰, R²⁵¹, R²⁵², R²⁵³, R²⁵⁴, R²⁵⁶, R²⁵⁷, R²⁵⁸, R²⁵⁹, R²⁶⁰, R²⁶², R²⁶³, R²⁶⁴, R²⁶⁵, R²⁶⁶, R²⁶⁷, R²⁶⁸, R²⁶⁹, R²⁷², R²⁷³, R²⁷⁴, R²⁷⁵, R²⁷⁶, R²⁷⁷, R²⁷⁸, R²⁷⁹, R²⁸⁰, R²⁸², R²⁸³, R²⁸⁵, R²⁸⁶, R²⁸⁸, R²⁸⁹, R²⁹¹, R²⁹², R²⁹³, R²⁹⁴, R²⁹⁶, R²⁹⁷, R²⁹⁹, R³⁰⁰, R³⁰¹, R³⁰², R³⁰³, R³⁰⁴, R³⁰⁵, R³⁰⁶, R³⁰⁷, R³⁰⁸, R³⁰⁹, R³¹¹, R³¹², R³¹³, R³¹⁴, and R³¹⁵ are each hydrogen;
R²⁴¹ and R²⁴⁵ are each independently a hydrogen or methyl group;
R²⁴³, R²⁶¹, R²⁸⁴, R²⁹⁰, R²⁹⁵, R³¹⁰ are each independently a hydrogen or OH;
R²³¹ is —(CO)NH₂, an ethyl group substituted with a hydroxyl group, or an isopropyl group;
R²⁴⁰ and R²⁵⁵ are each independently —CO₂H or —NH₂;
R²⁷⁰ and R²⁷¹ are each independently —CO₂H;
R²⁸¹ is —SH or —NH₂;
R²⁸⁷ is —(CO)NH₂ or —OH;
R²⁹⁸ is —NH₂, —CO₂H, or —SH;
R³¹⁶ is —NH₂;
R³¹⁷ is hydrogen or —NH₂;
JJ, KK, LL, MM, NN, QQ, and RR are each independently —CH₂—.

In any of the embodiments herein, the peptoid of Formula II may be selected from the peptoids recited in Table C. Note that in Table C, peptoids of the present technology include those where ηhomoarginine (ηHar) or η2-amino-4-guandinyl butyric acid (ηAgb) are used where ηArg is indicated.

TABLE C

ηArg-ηDmt-ηLys-ηPhe-ηGlu-Kys-Gly-NH₂
ηPhe-ηArg-ηPhe-ηLys-ηGlu-Kys-Gly-NH₂
ηPhe-ηArg-ηDmt-ηLys-ηGlu-Kys-Gly-NH₂
ηAla-ηPhe-ηArg-ηTyr-ηLys-ηTrp-ηHis-ηTyr-Gly-Phe
ηAsp-ηTrp-ηLys-ηTyr-ηHis-ηPhe-ηArg-Gly-ηLys-NH₂
ηHis-ηGlu-ηLys-ηTyr-ηPhe-ηArg
ηHis-ηLys-ηTyr-ηPhe-ηGlu-ηAsp-ηAsp-ηHis-ηLys-ηArg-
ηTrp-NH₂
ηLys-ηGln-ηTyr-ηArg-ηPhe-ηTrp-NH₂
ηLys-ηTrp-ηTyr-ηArg-ηAsn-ηPhe-ηTyr-ηHis-NH₂
ηPhe-ηArg-ηLys-ηTrp-ηTyr-ηArg-ηHis
ηThr-Gly-ηTyr-ηArg-ηHis-ηPhe-ηTrp-ηHis-ηLys
ηTrp-ηLys-ηPhe-ηAsp-ηArg-ηTyr-ηHis-ηLys
ηVal-ηLys-ηHis-ηTyr-ηPhe-ηSer-IITyr-ηArg-NH₂
Gly-ηPhe-ηLys-ηTyr-ηHis-ηArg-ηTyr-NH₂
ηAsp-ηTrp-ηLys-ηTyr-ηHis-ηPhe-ηArg-Gly-ηLys-NH₂
ηHis-ηLys-ηTyr-ηPhe-ηGlu-ηAsp-ηHis-ηLys-ηArg-ηTrp-NH₂
H-ηPhe-ηArg-ηPhe-ηLys-ηGlu-Kys-Gly-NH₂
ηPhe-ηArg-ηPhe-ηLys-ηGlu-Kys-Gly
H-ηArg-ηDmt-ηLys-ηPhe-ηSar-Gly-Wys-NH₂

In some embodiments of the present technology, the peptoid may be of Formula III:

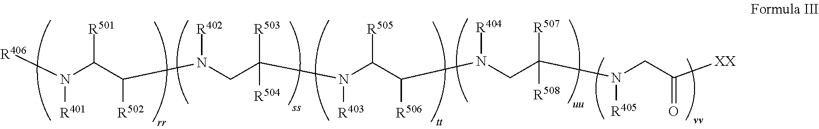

wherein:
XX is —N(R⁴⁰⁸)(R⁴⁰⁹) or —O—R⁴¹⁰;
R⁴⁰¹ is

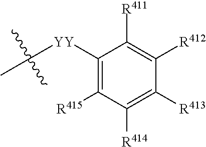

R⁴⁰⁷,
R⁴⁰² is

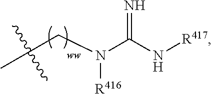

or optionally R⁴⁰⁷ if rr is 0;
R⁴⁰³ is

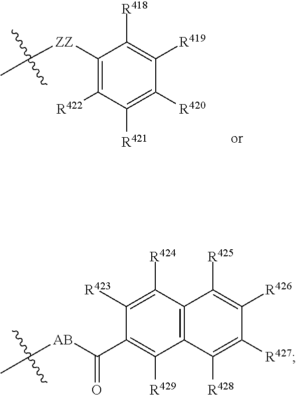

R⁴⁰⁴ is

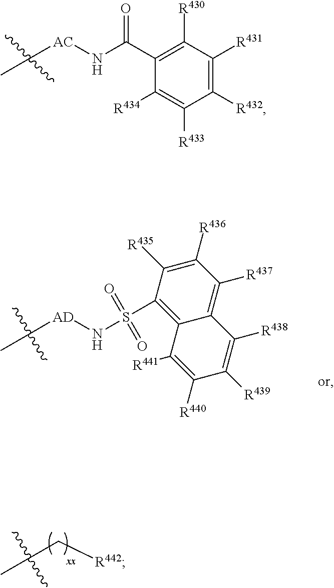

R⁴⁰⁵ is

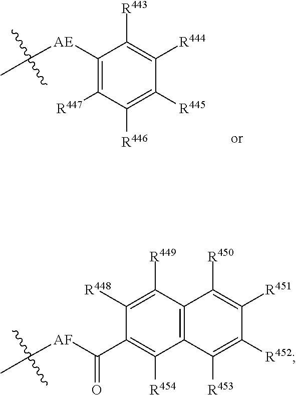

wherein
   R⁴⁰⁶, R⁴⁰⁷, R⁴⁰⁸, R⁴⁰⁹, and R⁴¹⁰ are each independently a hydrogen or substituted or unsubstituted C₁-C₆ alkyl, C₂-C₆ alkenyl, C₂-C₆ alkynyl, saturated or unsaturated cycloalkyl, cycloalkylalkyl, aryl, aralkyl, 5- or 6-membered saturated or unsaturated heterocylyl, heterobicyclyl, heteroaryl, or amino protecting group; or R⁴⁰⁶ and R⁴⁰⁷ together or R⁴⁰⁸ and R⁴⁰⁹ together form a 3-, 4-, 5-, 6-, 7-, or 8-member substituted or unsubstituted heterocycyl ring;
   R⁵⁰¹ and R⁵⁰² are each independently a hydrogen or substituted or unsubstituted C₁-C₆ alkyl group; or together R⁵⁰¹ and R⁵⁰² are C═O;
   R⁵⁰³ and R⁵⁰⁴ are each independently a hydrogen or substituted or unsubstituted C₁-C₆ alkyl group; or together R⁵⁰³ and R⁵⁰⁴ are C═O;
   R⁵⁰⁵ and R⁵⁰⁶ are each independently a hydrogen or substituted or unsubstituted C₁-C₆ alkyl group; or together R⁵⁰⁵ and R⁵⁰⁶ are C═O;
   R⁵⁰⁷ and R⁵⁰⁸ are each independently a hydrogen or substituted or unsubstituted C₁-C₆ alkyl group; or together R⁵⁰⁷ and R⁵⁰⁸ are C═O;
   R⁴¹¹, R⁴¹², R⁴¹³, R⁴¹⁴, R⁴¹⁵, R⁴¹⁸, R⁴¹⁹, R⁴²⁰, R⁴²¹, R⁴²², R⁴²³, R⁴²⁴, R⁴²⁵, R⁴²⁶, R⁴²⁷, R⁴²⁸, R⁴²⁹, R⁴³⁰, R⁴³¹, R⁴³², R⁴³³, R⁴³⁴, R⁴³⁵, R⁴³⁶, R⁴³⁷, R⁴³⁸, R⁴³⁹, R⁴⁴⁰, R⁴⁴¹, R⁴⁴³, R⁴⁴⁴, R⁴⁴⁵, R⁴⁴⁶, R⁴⁴⁷, R⁴⁴⁸, R⁴⁴⁹, R⁴⁵⁰, R⁴⁵¹, R⁴⁵², R⁴⁵³, and R⁴⁵⁴ are each independently a hydrogen, deuterium, amino, amido, —NO₂, —CN, —OR^{e}, —SR^{e}, NR^{e}R^{e}, —F, —Cl, —Br, —I, or a substituted or unsubstituted C₁-C₆ alkyl, C₁-C₆ alkoxy, —C(O)-alkyl, —C(O)-aryl, —C(O)-aralkyl, —C(O)₂R^{e}, C₁-C₄ alkylamino, C₁-C₄ dialkylamino, or perhaloalkyl group;
   R⁴¹⁶ and R⁴¹⁷ are each independently a hydrogen, —C(O)R^{e}, or a substituted or unsubstituted C₁-C₆ alkyl;
   R⁴⁴² is a hydrogen, —OR^{e}, —SR^{e}, —NR^{e}R^{e}, —NR^{e}R^{f}, —CO₂R^{e}, —C(O)NR^{e}R^{e}, —NR^{e}C(O)R^{e}, —NR^{e}C(NH)NH₂, —NR^{e}-dansyl, enamine, imine, or a substituted or unsubstituted alkyl, heterocyclyl, aryl, heteroaryl, or aralkyl group;
   YY, ZZ, and AE are each independently absent, —NH(CO)—, or —CH₂—;
   AB, AC, AD, and AF are each independently absent or C₁-C₆ alkylene group;
   R^{e} at each occurrence is independently a hydrogen or a substituted or unsubstituted C₁-C₆ alkyl group;
   R^{f} at each occurrence is independently a C₁-C₆ alkylene-NR^{e}-dansyl or C₁-C₆ alkylene-NR^{e}-anthraniloyl group;
   rr, ss, and vv are each independently 0 or 1; tt and uu are each 1
      with the proviso that rr+ss+tt+uu+vv equals 4 or 5; and
   ww and xx are each independently 1, 2, 3, 4, or 5.
   with the proviso that when vv is 0, then uu is 1 and together R⁵⁰⁷ and R⁵⁰⁸ are C═O.

In any embodiment herein of peptoids of Formula III, it may be that
R⁴⁰⁶ is a hydrogen, substituted or unsubstituted C₁-C₆ alkyl group,

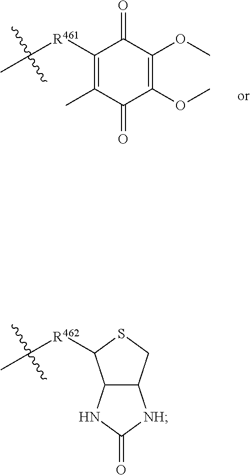

where R⁴⁶¹ is a —C₁-C₁₀ alkylene-CO₂— or —CO₂—C₁-C₁₀ alkylene-CO₂—; and
   R⁴⁶² is C₁-C₁₀ alkylene or C₁-C₁₀ alkylene-CO₂—;
R⁴⁰⁷, R⁴⁰⁸, R⁴⁰⁹, and R⁴¹⁰ are each independently a hydrogen or substituted or unsubstituted C₁-C₆ alkyl group;
R⁴⁵⁵ and R⁴⁶⁰ are each independently a hydrogen, —C(O)—C₁-C₆ alkyl, or methyl group;
R⁴⁵⁶ and R⁴⁵⁷ are each a hydrogen or together R⁴⁵⁶ and R⁴⁵⁷ are C═O;
R⁴⁵⁸ and R⁴⁵⁹ are each a hydrogen or together R⁴⁵⁸ and R⁴⁵⁹ are C═O;
R⁴¹⁶ and R⁴¹⁷ are each independently a hydrogen or —C(O)R^{e};
R⁴¹¹, R⁴¹², R⁴¹³, R⁴¹⁴, R⁴¹⁵, R⁴¹⁸, R⁴¹⁹, R⁴²⁰, R⁴²¹, R⁴²², R⁴⁴³, R⁴⁴⁴, R⁴⁴⁵, R⁴⁴⁶, and R⁴⁴⁷ are each independently a hydrogen, deuterium, methyl, or —OR^{e} group;
R⁴²³, R⁴²⁴, R⁴²⁵, R⁴²⁶, R⁴²⁷, R⁴²⁸, R⁴²⁹, R⁴³⁰, R⁴³¹, R⁴³², R⁴³³, R⁴³⁴, R⁴³⁵, R⁴³⁶, R⁴³⁷, R⁴³⁸, R⁴³⁹, R⁴⁴⁰, R⁴⁴¹, R⁴⁴⁸, R⁴⁴⁹, R⁴⁵⁰, R⁴⁵¹, R⁴⁵², R⁴⁵³, and R⁴⁵⁴ are each independently a hydrogen, NR^{e}R^{e}, or substituted or unsubstituted C₁-C₆ alkyl group;
R⁴⁴² is a —NR^{e}R^{e};
YY, ZZ, and AE are each independently absent or —CH₂—.

In any embodiment herein of peptoids of Formula III, it may be that
R⁴⁰⁶ is

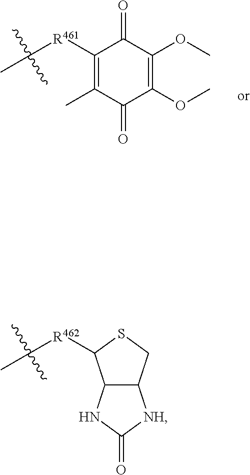

hydrogen, or methyl, where R⁴⁶¹ is a —(CH₂)₃—CO₂—, —(CH₂)₉—CO₂—, or —CO₂—(CH₂)₂—CO2- and R⁴⁶² is —(CH₂)₄—CO₂—;
R⁴⁰⁷, R⁴⁰⁸, R⁴⁰⁹, and R⁴¹⁰ are each a hydrogen or methyl group;
R⁴⁵⁵ and R⁴⁶⁰ are each independently a hydrogen, —C(O)CH₃, or methyl group;
R⁴⁵⁶ and R⁴⁵⁷ are each a hydrogen or together R⁴⁵⁶ and R⁴⁵⁷ are C═O;
R⁴⁵⁸ and R⁴⁵⁹ are each a hydrogen or together R⁴⁵⁸ and R⁴⁵⁹ are C═O;
R⁴¹⁶ and R⁴¹⁷ are each independently a hydrogen or —C(O)CH₃;
R⁴²⁶, R⁴³⁸, and R⁴⁵¹ are each —N(CH₃)₂;
R⁴³⁴ and R⁴⁴² are each —NH₂;
R⁴²³, R⁴²⁴, R⁴²⁵, R⁴²⁷, R⁴²⁸, R⁴²⁹, R⁴³⁰, R⁴³¹, R⁴³², R⁴³³, R⁴³⁵, R⁴³⁶, R⁴³⁷, R⁴³⁹, R⁴⁴⁰, R⁴⁴¹, R⁴⁴³, R⁴⁴⁴, R⁴⁴⁵, R⁴⁴⁶, R⁴⁴⁷, R⁴⁴⁸, R⁴⁴⁹, R⁴⁵⁰, R⁴⁵², R⁴⁵³, and R⁴⁵⁴ are each hydrogen;
R⁴¹², R⁴¹⁴, R⁴¹⁹, and R⁴²¹ are each independently hydrogen or deuterium;
R⁴¹¹, R⁴¹⁵, R⁴¹⁸, and R⁴²² are each independently hydrogen, deuterium, or methyl;
R⁴¹³ and R⁴²⁰ are each independently hydrogen, deuterium, or OR^{e};
YY, ZZ, and AE are each independently —CH₂—;
AB, AC, AD, and AF are each —CH₂— or a butylene group; and
ww and xx are each independently 3 or 4.

In any of the embodiments herein, the peptoid may be selected from the peptoids shown in Table D. Peptoids of the present technology further include those where ηhomoarginine (ηHar) or η2-amino-4-guandinyl butyric acid (ηAgb) are used where ηArg is indicated in Table D.

TABLE D

6-Butyric acid CoQ0-ηPhe-ηArg-ηPhe-ηLys-NH₂
6-Decanoic acid CoQ0-ηPhe-ηArg-ηPhe-ηhys-NH₂
H-ηN2-acetylarginine-ηDmt-ηLys-ηPhe-NH₂
H-ηN8-acetylarginine-ηDmt-ηLys-ηPhe-NH₂
H-ηN7-acetylarginine-ηDmt-ηLys-ηPhe-NH₂
H-ηPhe(d5)- ηArg-ηPhe(d5)-ηLys-NH₂
Succinic monoester CoQ0-ηPhe-ηArg-ηPhe-ηLys-HN₂
ηDmt-ηArg-ηPhe-η(atn)Dap-NH₂
ηDmt-ηArg-ηPhe-η(dns)Dap-NH₂
ηDmt-ηArg-ηAld-ηLys-NH₂
ηDmt-ηArg-ηPhe-ηLys-ηAld-NH₂
Bio-η2′6′Dmt-ηArg-ηPhe-ηLys-NH₂
η2′6′Dmt-ηArg-ηPhe-ηdnsDap-NH₂
η2′6′Dmt-ηArg-ηPhe-ηatnDap-NH₂
H-ηArg-Ψ[CH₂—NH]ηDmt-ηLys-ηPhe-NH₂
H-ηArg-ηDmt-Ψ[CH₂—NH]Lys-ηPhe-NH₂
H-ηArg-ηDmt-ηLys-Ψ[CH₂—NH]ηPhe-NH₂
H-ηArg-ηDmt-Ψ[CH₂—NH]Lys-Ψ[CH₂—NH]ηPhe-NH₂

Bio = biotin
CoQ0 = coenzyme QO

In any of the above embodiments of peptoids, the peptoid may be one of the peptoids recited in Table E. Peptoids of the present technology further include those where ηhomoarginine (ηHar) or η2-amino-4-guandinyl butyric acid (ηAgb) are used where ηArg is indicated in Table E.

TABLE E

ηArg-ηLeu-ηTyr-ηPhe-ηLys-ηGlu-ηLys-ηArg-ηTrp-ηLys-
ηPhe-ηTyr-ηArg-Gly
ηAsp-ηArg-ηPhe-ηCys-ηPhe-ηArg-ηLys-ηTyr-ηArg-ηTyr-
ηTrp-ηHis-ηTyr-ηPhe-ηLys-ηPhe
ηGlu-ηAsp-ηLys-ηArg-ηHis-ηPhe-ηPhe-ηVal-ηTyr-ηArg-
ηTyr-ηTyr-ηArg-ηHis-ηPhe-NH₂
ηGlu-ηArg-ηLys-ηTyr-ηVal-ηPhe-ηHis-ηTrp-ηArg-Gly-ηTyr-
ηArg-ηMet-NH₂
Gly-ηAla-ηLys-ηPhe-ηLys-ηGlu-ηArg-ηTyr-ηHis-ηArg-
ηArg-ηAsp-ηTyr-ηTrp-ηHis-ηTrp-ηHis-ηLys-ηAsp
ηHis-ηTyr-ηArg-ηTrp-ηLys-ηPhe-ηAsp-ηAla-ηArg-ηCys-
ηTyr-ηHis-ηPhe-ηLys-ηTyr-ηHis-ηSer-NH₂
ηPhe-ηPhe-ηTyr-ηArg-ηGlu-ηAsp-ηLys-ηArg-ηArg-ηHis-
ηPhe-NH₂
ηPhe-ηTyr-ηLys-ηArg-ηTrp-ηHis-ηLys-ηLys-ηGlu-ηArg-
ηTyr-ηThr
ηThr-ηTyr-ηArg-ηLys-ηTrp-ηTyr-ηGlu-ηAsp-ηLys-ηArg-
ηHis-ηPhe-ηTyr-Gly-ηVal-ηIle-ηHis-ηArg-ηTyr-ηLys-NH₂
ηTyr-ηAsp-ηLys-ηTyr-ηPhe-ηLys-ηArg-ηPhe-Pro-ηTyr-His-
ηLys
ηTyr-ηHis-ηPhe-ηArg-ηAsp-ηLys-ηArg-ηHis-ηTrp-ηHis-
ηPhe
ηPhe-ηTyr-ηLys-ηArg-ηTrp-ηHis-ηLys-ηLys-ηGlu-ηArg-
ηTyr-ηThr
ηTyr-ηAsp-ηLys-ηTyr-ηPhe-ηLys-ηArg-ηPhe-Pro-ηTyr-ηHis-
ηLys
ηGlu-ηArg-ηLys-ηTyr-ηVal-ηPhe-ηHis-ηTrp-ηArg-Gly-ηTyr-
ηArg-ηMet-NH₂
ηArg-ηLeu-ηTyr-ηPheη-Lys-ηGlu-ηLys-ηArg-ηTrp-ηLys-
ηPhe-ηTyr-ηArg-Gly
Gly-ηAla-ηLys-ηPhe-ηLys-ηGlu-ηArg-ηTyr-ηHis-ηArg-
ηArg-ηAsp-ηTyr-ηTrp-ηHis-ηTrp-ηHis-ηLys-ηAsp
Gly-ηAla-ηLys-ηPhe-ηLys-ηGlu-ηArg-ηTyr-ηHis-ηArg-
ηArg-ηAsp-ηTyr-ηTrp-ηHis-ηTrp-ηHis-ηLys-ηAsp

In some embodiments, the peptoid is defined by Formula IV:

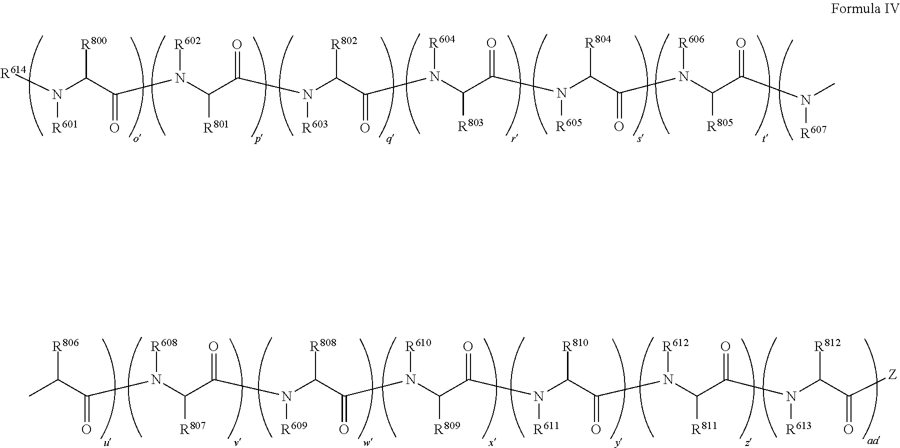

wherein:
Z is —N(R⁶¹⁶)(R⁶¹⁷) or —O—R⁶¹⁸;
R⁶⁰¹ is

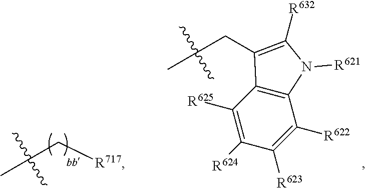

or together with R⁸⁰⁰ is a substituted or unsubstituted C₃ alkyenyl group, or is R⁶¹⁵, provided that when R⁸⁰⁰ is not hydrogen or together with R⁶⁰¹ a substituted or unsubstituted C₃ alkyenyl group then R⁶⁰¹ is hydrogen;
R⁶⁰² is

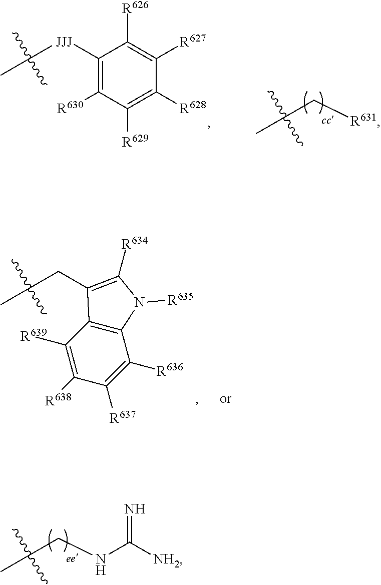

or together with R⁸⁰¹ is a substituted or unsubstituted C₃ alkyenyl group, or optionally R⁶¹⁵ if o′ is 0, provided that when R⁸⁰¹ is not hydrogen or together with R⁶⁰² a substituted or unsubstituted C₃ alkyenyl group then R⁶⁰² is hydrogen;
R⁶⁰³ is

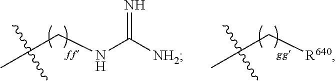

or hydrogen, or together with R⁸⁰² is a substituted or unsubstituted C₃ alkyenyl group, or optionally R⁶¹⁵ if o′ and p′ are each 0, with the proviso that when R⁸⁰² is not hydrogen or together with R⁶⁰³ a substituted or unsubstituted C₃ alkyenyl group then R⁶⁰³ is hydrogen;
R⁶⁰⁴ is

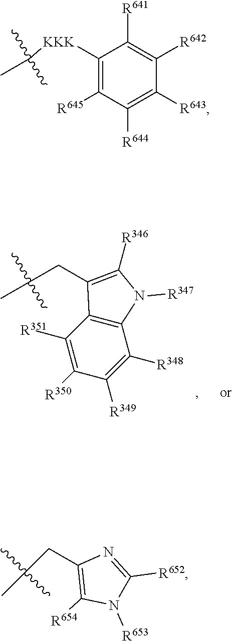

or together with R⁸⁰³ is a substituted or unsubstituted C₃ alkyenyl group, or optionally R⁶¹⁵ if o′, p′, and q′ are each 0, with the proviso that when R⁸⁰³ is not hydrogen or together with R⁶⁰⁴ a substituted or unsubstituted C₃ alkyenyl group then R⁶⁰⁴ is hydrogen;
R⁶⁰⁵ is

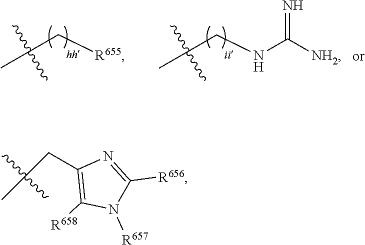

or together with R⁸⁰⁴ is a substituted or unsubstituted C₃ alkyenyl group, or optionally R⁶¹⁵ if o′, p′, q′, and r′ are each 0, with the proviso that when R⁸⁰⁴ is not hydrogen or together with R⁶⁰⁵ a substituted or unsubstituted C₃ alkyenyl group then R⁶⁰⁵ is hydrogen;
R⁶⁰⁶ is

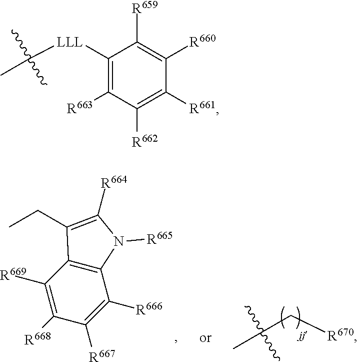

or together with R⁸⁰⁵ is a substituted or unsubstituted C₃ alkyenyl group, or optionally R⁶¹⁵ if o′, p′, q′, r′, and s′ are each 0, with the proviso that when R⁸⁰⁵ is not hydrogen or together with R⁶⁰⁶ a substituted or unsubstituted C₃ alkyenyl group then R⁶⁰⁶ is hydrogen;
R⁶⁰⁷ is

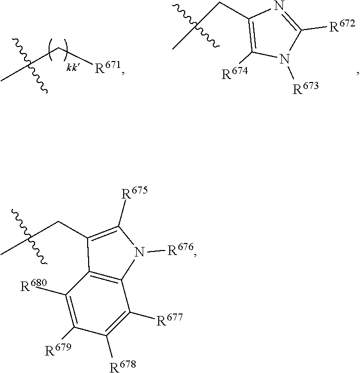

or hydrogen, or together with R⁸⁰⁶ is a substituted or unsubstituted C₃ alkyenyl group, or optionally R⁶¹⁵ if o′, p′, q′, r′, s′, and t′ are each 0, with the proviso that when R⁸⁰⁶ is not hydrogen or together with R⁶⁰⁷ a substituted or unsubstituted C₃ alkyenyl group then R⁶⁰⁷ is hydrogen;
R⁶⁰⁸ is

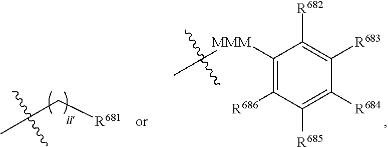

or R⁶⁸⁵, or together with R⁸⁰⁷ is a substituted or unsubstituted C₃ alkyenyl group, or optionally R⁶¹⁵ if o′, p′, q′, r′, s′, t′, and u′ are each 0, with the proviso that when R⁸⁰⁷ is not hydrogen or together with R⁶⁰⁸ a substituted or unsubstituted C₃ alkyenyl group then R⁶⁰⁸ is hydrogen;
R⁶⁰⁹ is

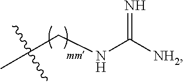

or together with R⁸⁰⁸ is a substituted or unsubstituted C₃ alkyenyl group, or optionally R⁶¹⁵ if o′, p′, q′, r′, s′, t′, u′, and v′ are each 0, with the proviso that when R⁸⁰⁸ is not hydrogen or together with R⁶⁰⁹ a substituted or unsubstituted C₃ alkyenyl group then R⁶⁰⁹ is hydrogen;
R⁶¹⁰ is

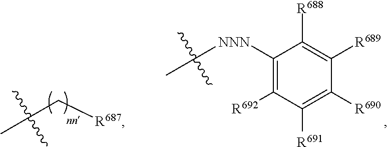

or hydrogen, or together with R⁸⁰⁹ is a substituted or unsubstituted C₃ alkyenyl group, or optionally R⁶¹⁵ if o′, p′, q′, r′, s′, t′, u′, v′, and w′ are each 0, with the proviso that when R⁸⁰⁹ is not hydrogen or together with R⁶¹⁰ a substituted or unsubstituted C₃ alkyenyl group then R⁶¹⁰ is hydrogen;
R⁶¹¹ is

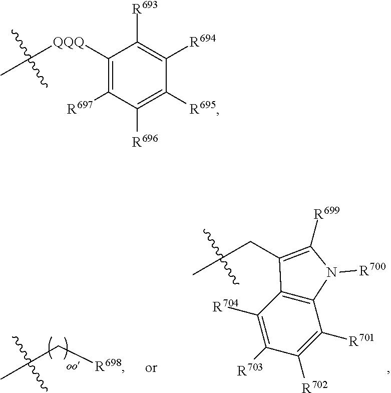

or together with R⁸¹⁰ is a substituted or unsubstituted C₃ alkyenyl group, with the proviso that when R⁸¹⁰ is not hydrogen or together with R⁶¹¹ a substituted or unsubstituted C₃ alkyenyl group then R⁶¹¹ is hydrogen;
R⁶¹² is

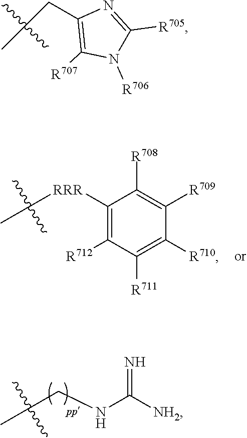

or together with R⁸¹¹ is a substituted or unsubstituted C₃ alkyenyl group, with the proviso that when R⁸¹¹ is not hydrogen or together with R⁶¹² a substituted or unsubstituted C₃ alkyenyl group then R⁶¹² is hydrogen;
R⁶¹³ is

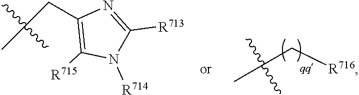

or together with R⁸¹² is a substituted or unsubstituted C₃ alkyenyl group, with the proviso that when R⁸¹² is not hydrogen or together with R⁶¹³ a substituted or unsubstituted C₃ alkyenyl group then R⁶¹³ is hydrogen;
one or two of R⁸⁰⁰, R⁸⁰¹, R⁸⁰², R⁸⁰³, R⁸⁰⁴, R⁸⁰⁵, R⁸⁰⁶, R⁸⁰⁷, R⁸⁰⁸, R⁸⁰⁹, R⁸¹⁰, R⁸¹¹, and R⁸¹² are each independently the aforementioned substituted or unsubstituted C₃ alkyenyl group,

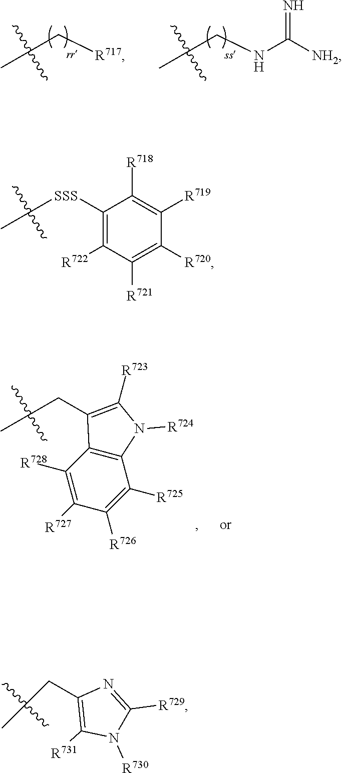

and the remaining R⁸⁰⁰, R⁸⁰¹, R⁸⁰², R⁸⁰³, R⁸⁰⁴, R⁸⁰⁵, R⁸⁰⁶, R⁸⁰⁷, R⁸⁰⁸, R⁸⁰⁹, R⁸¹⁰, R⁸¹¹, and R⁸¹² are each hydrogen,
   wherein
      R⁶¹⁴, R⁶¹⁵, R⁶¹⁶, R⁶¹⁷, and R⁶¹⁸ are each independently a hydrogen or substituted or unsubstituted C₁-C₆ alkyl, C₂-C₆ alkenyl, C₂-C₆ alkynyl, saturated or unsaturated cycloalkyl, cycloalkylalkyl, aryl, aralkyl, 5- or 6-membered saturated or unsaturated heterocylyl, heteroaryl, or amino protecting group; or R⁶¹⁴ and R⁶¹⁵ together or R⁶¹⁶ and R⁶¹⁷ together form a 3, 4, 5, 6, 7, or 8 membered substituted or unsubstituted heterocycyl ring;
      R⁶²², R⁶²³, R⁶²⁴, R⁶²⁵, R⁶²⁶, R⁶²⁷, R⁶²⁸, R⁶²⁹, R⁶³⁰, R⁶³², R⁶³⁴, R⁶³⁶, R⁶³⁷, R⁶³⁸, R⁶³⁹, R⁶⁴¹, R⁶⁴², R⁶⁴³, R⁶⁴⁴, R⁶⁴⁵, R⁶⁴⁶, R⁶⁴⁸, R⁶⁴⁹, R⁶⁵⁰, R⁶⁵¹, R⁶⁵², R⁶⁵⁴, R⁶⁵⁶, R⁶⁵⁸, R⁶⁵⁹, R⁶⁶⁰, R⁶⁶¹, R⁶⁶², R⁶⁶³, R⁶⁶⁴, R⁶⁶⁶, R⁶⁶⁷, R⁶⁶⁸, R⁶⁶⁹, R⁶⁷², R⁶⁷⁴, R⁶⁷⁵, R⁶⁷⁷, R⁶⁷⁸, R⁶⁷⁹, R⁶⁸⁰, R⁶⁸², R⁶⁸³, R⁶⁸⁴, R⁶⁸⁵, R⁶⁸⁶, R⁶⁸⁸, R⁶⁸⁹, R⁶⁹⁰, R⁶⁹¹, R⁶⁹², R⁶⁹³, R⁶⁹⁴, R⁶⁹⁵, R⁶⁹⁶, R⁶⁹⁷, R⁶⁹⁹, R⁷⁰¹, R⁷⁰², R⁷⁰³, R⁷⁰⁴, R⁷⁰⁵, R⁷⁰⁷, R⁷⁰⁸, R⁷⁰⁹, R⁷¹⁰, R⁷¹¹, R⁷¹², R⁷¹³, R⁷¹⁵, R⁷¹⁸, R⁷¹⁹, R⁷²⁰, R⁷²¹, R⁷²², R⁷²³, R⁷²⁵, R⁷²⁶, R⁷²⁷, R⁷²⁸, R⁷³⁰, and R⁷³¹ are each independently a hydrogen, amino, amido, —NO₂, —CN, —OR^{c}, SR^{c}, —NR^{c}R^{c}, —F, —Cl, —Br, —I, or a substituted or unsubstituted C₁-C₆ alkyl, C₁-C₆ alkoxy, —C(O)-alkyl, —C(O)-aryl, —C(O)-aralkyl, —C(O)₂R^{c}, C₁-C₄ alkylamino, C₁-C₄ dialkylamino, or perhaloalkyl group;
      R⁶²¹, R⁶³⁵, R⁶⁴⁷, R⁶⁵³, R⁶⁵⁷, R⁶⁶⁵, R⁶⁷³, R⁶⁷⁶, R⁷⁰⁰, R⁷⁰⁶, R⁷¹⁴, R⁷²⁴, and R⁷²⁹ are each independently a hydrogen or substituted or unsubstituted C₁-C₆ alkyl group;
      R⁶¹⁶, R⁶¹⁷, R631, R⁶⁴⁰, R⁶⁵⁵, R⁶⁷⁰, R⁶⁷¹, R⁶⁸¹, R⁶⁸⁷, R⁶⁹⁸, and R⁷¹⁷ are each independently a hydrogen, —OR^{g}, —SR^{g}, —NR^{g}R^{g}, —NR^{g}R^{h}, —CO₂R^{g}, —(CO)NR^{g}R^{g}, —NR^{g}(CO)R^{g}, —NR^{g}C(NH)NH₂, —NR^{g}-dansyl, enamine, imine, or a substituted or unsubstituted alkyl, heterocyclyl, aryl, heteroaryl, or aralkyl group;
      JJJ, KKK, LLL, MMM, NNN, QQQ, RRR, and SSS are each independently absent, —NH(CO)—, or —CH₂—;
      R^{g} at each occurrence is independently a hydrogen or a substituted or unsubstituted C₁-C₆ alkyl group;
      R^{h} at each occurrence is independently a C₁-C₆ alkylene-NR^{c}-dansyl or C₁-C₆ alkylene-NR^{c}-anthraniloyl group;
      o′, p′, q′, r′, s′, t′, u′, v′, w′, x′, y′, z′, and aa′ are each independently 0 or 1,
         with the proviso that o′+p′+q′+r′+s′+t′+u′+v′+w′+x′+y′+z′+aa′ equals 4, 5, 6, 7, 8, 9, 10, or 11;
      bb′, cc′, ee′, ff′, gg′, hh′, ii′, jj′, kk′, ll′, mm′, nn′, oo′, pp′, qq′, rr′, and ss' are each independently 1, 2, 3, 4, or 5.

In one embodiment, the neutral-cationic peptoids of the present technology have a core structural motif of alternating neutral and cationic peptoid monomers. For example, the peptoid may be a tetrapeptoid defined by any of Formulas A to F set forth below:

Neutral-Cationic-Neutral-Cationic (Formula A)

Cationic-Neutral-Cationic-Neutral (Formula B)

Neutral-Neutral-Cationic-Cationic (Formula C)

Cationic-Cationic-Neutral-Neutral (Formula D)

Neutral-Cationic-Cationic-Neutral (Formula E)

Cationic-Neutral-Neutral-Cationic (Formula F)

wherein, Neutral is a residue selected from the group consisting of: ηPhe (ηF), η2,6-DMF, ηTyr (ηY), η2,6-DMT, and ηTrp (ηW). In some embodiments, the ηPhe, η2,6-DMF, ηTyr, η2,6-DMT, and/or ηTrp residue may be substituted with a saturated analog, e.g., ηCyclohexylalanine (ηCha) for ηPhe. In some embodiments, Cationic is a residue selected from the group consisting of: ηArg (ηR), ηLys (ηK), and ηHis (ηH).

The peptoid monomers may be the peptoid monomer analogues of naturally occurring amino acids. Thus, the peptoid monomers include peptoid monomer analogues of the eighteen most common amino acids normally found in mammalian proteins that are not glycine (Gly) or proline (Pro), i.e., ηalanine (ηAla), ηarginine (ηArg), ηasparagine (ηAsn), ηaspartic acid (ηAsp), ηcysteine (ηCys), ηglutamine (ηGln), ηglutamic acid (ηGlu), ηhistidine (ηHis), ηisoleucine (ηIle), ηleucine (ηLeu), ηlysine (ηLys), ηmethionine (ηMet), ηphenylalanine (ηPhe), ηserine (ηSer), ηthreonine (ηThr), ηtryptophan, (ηTrp), ηtyrosine (ηTyr), and ηvaline (ηVal).

Other naturally occurring amino acids include, for example, amino acids that are synthesized in metabolic processes not associated with protein synthesis. For example, the amino acids ornithine and citrulline are synthesized in mammalian metabolism during the production of urea. Thus, the peptoid monomers include the peptoid analogue of ornithine (i.e., ηornithine; ηOrn) and the peptoid monomer analogue of citrulline (i.e., ηcitrulline, ηCit).

The peptoids useful in the present technology may also include one or more non-naturally occurring amino acids or peptoid monomers analogous to one or more non-naturally occurring amino acids.

The non-naturally occurring amino acid or peptoid monomers analogous to one or more non-naturally occurring amino acids may be present at any position in the peptoid. For example, the non-naturally occurring amino acid can be at the N terminus, the C-terminus, or at any position between the N-terminus and the C-terminus.

The non-natural amino acids may, for example, comprise alkyl, aryl, or alkylaryl groups. Some examples of alkyl amino acids include α-aminobutyric acid, β-aminobutyric acid, γ-aminobutyric acid, δ-aminovaleric acid, and ε-aminocaproic acid. Some examples of aryl amino acids include ortho-, meta, and para-aminobenzoic acid. Some examples of alkylaryl amino acids include ortho-, meta-, and para-aminophenyl acetic acid, and γ-phenyl-β-aminobutyric acid.

Non-naturally occurring amino acids also include derivatives of naturally occurring amino acids. The derivatives of naturally occurring amino acids may, for example, include the addition of one or more chemical groups to the naturally occurring amino acid.

For example, one or more chemical groups can be added to one or more of the 2′, 3′, 4′, 5′, or 6′ position of the aromatic ring of a phenylalanine or tyrosine residue, or the 4′, 5′, 6′, or 7′ position of the benzo ring of a tryptophan residue. The group can be any chemical group that can be added to an aromatic ring. Some examples of such groups include branched or unbranched C₁-C₄ alkyl, such as methyl, ethyl, n-propyl, isopropyl, butyl, isobutyl, or t-butyl, C₁-C₄ alkyloxy (i.e., alkoxy), amino, C₁-C₄ alkylamino and C₁-C₄ dialkylamino (e.g., methylamino, dimethylamino), nitro, hydroxyl, halo (i.e., fluoro, chloro, bromo, or iodo). Some specific examples of non-naturally occurring derivatives of naturally occurring amino acids include norvaline (Nva), norleucine (Nle), and hydroxyproline (Hyp).

Another example of a modification of an amino acid in a peptoid useful in the present methods is the derivatization of a carboxyl group of an aspartic acid, an ηaspatric acid, a glutamic acid, or a ηglutamic acid residue of the peptoid. One example of derivatization is amidation with ammonia or with a primary or secondary amine, e.g., methylamine, ethylamine, dimethylamine or diethylamine. Another example of derivatization includes esterification with, for example, methyl or ethyl alcohol.

Another such modification includes derivatization of an amino group of a lysine, ηlysine, arginine, ηarginine, histidine, or ηhistidine residue. For example, such amino groups can be alkylated or acylated. Some suitable acyl groups include, for example, a benzoyl group or an alkanoyl group comprising any of the C₁-C₄ alkyl groups mentioned above, such as an acetyl or propionyl group.

In some embodiments, the non-naturally occurring amino acids are resistant, and in some embodiments insensitive, to common proteases. Examples of non-naturally occurring amino acids that are resistant or insensitive to proteases include the dextrorotatory (D-) form of any of the above-mentioned naturally occurring L-amino acids, as well as L- and/or D non-naturally occurring amino acids. The D-amino acids do not normally occur in proteins, although they are found in certain peptide antibiotics that are synthesized by means other than the normal ribosomal protein synthetic machinery of the cell, as used herein, the D-amino acids are considered to be non-naturally occurring amino acids.

In order to minimize protease sensitivity, the peptoids useful in the methods of the present technology should have less than two contiguous L-amino acids recognized by common proteases, irrespective of whether the amino acids are naturally or non-naturally occurring. In some embodiments, the peptoid has only D-amino acids, and no L-amino acids.

It is important that the neutral-cationic peptoids have a minimum number of net positive charges at physiological pH in comparison to the total number of monomer residues in the peptoid. The minimum number of net positive charges at physiological pH is referred to below as (pₘ). The total number of monomer residues in the peptoid is referred to below as (r).

The minimum number of net positive charges discussed below are all at physiological pH. The term “physiological pH” as used herein refers to the normal pH in the cells of the tissues and organs of the mammalian body. For instance, the physiological pH of a human is normally approximately 7.4, but normal physiological pH in mammals may be any pH from about 7.0 to about 7.8. As another example, physiological pH in the gastrointestinal tract of a human may be any pH from about 2.0 to about 8.0.

Typically, a peptoid has a positively charged N-terminal amino group and a negatively charged C-terminal carboxyl group. The charges cancel each other out at physiological pH. As an example of calculating net charge, the peptoid ηTyr-ηArg-ηPhe-ηLys-ηGlu-ηHis-ηTrp-ηArg has one negatively charged monomer residue (i.e., ηGlu) and four positively charged monomer residues (i.e., two ηArg residues, one ηLys, and one ηHis). Therefore, the above peptoid has a net positive charge of three.

In one embodiment, the neutral-cationic peptoids have a relationship between the minimum number of net positive charges at physiological pH (pₘ) and the total number of monomer residues (r) wherein 3 pₘ is the largest number that is less than or equal to r+1. In this embodiment, the relationship between the minimum number of net positive charges (pₘ) and the total number of monomer residues (r) is as follows:

TABLE 1
Monomer number and net positive charges (3pₘ ≤ p + 1)

| | | | | | | | | | | | | | | | | | | |
|---|---|---|---|---|---|---|---|---|---|---|---|---|---|---|---|---|---|---|
| (r) | 3 | 4 | 5 | 6 | 7 | 8 | 9 | 10 | 11 | 12 | 13 | 14 | 15 | 16 | 17 | 18 | 19 | 20 |
| (pₘ) | 1 | 1 | 2 | 2 | 2 | 3 | 3 | 3 | 4 | 4 | 4 | 5 | 5 | 5 | 6 | 6 | 6 | 7 |

In another embodiment, the neutral-cationic peptoids have a relationship between the minimum number of net positive charges (pₘ) and the total number of monomer residues (r) wherein 2 pₘ is the largest number that is less than or equal to r+1. In this embodiment, the relationship between the minimum number of net positive charges (pₘ) and the total number of monomer residues (r) is as follows:

TABLE 2
Monomer number and net positive charges (2pₘ ≤ p + 1)

| | | | | | | | | | | | | | | | | | | |
|---|---|---|---|---|---|---|---|---|---|---|---|---|---|---|---|---|---|---|
| (r) | 3 | 4 | 5 | 6 | 7 | 8 | 9 | 10 | 11 | 12 | 13 | 14 | 15 | 16 | 17 | 18 | 19 | 20 |
| (pₘ) | 2 | 2 | 3 | 3 | 4 | 4 | 5 | 5 | 6 | 6 | 7 | 7 | 8 | 8 | 9 | 9 | 10 | 10 |

In one embodiment, the minimum number of net positive charges (pₘ) and the total number of monomer residues (r) are equal. In another embodiment, the peptoids have three or four monomer residues and a minimum of one net positive charge, or a minimum of two net positive charges, or a minimum of three net positive charges.

The neutral-cationic peptoids may further have a minimum number of aromatic groups in comparison to the total number of net positive charges (pₜ). The minimum number of aromatic groups will be referred to below as (a). Naturally-occurring amino acids and peptoid monomer analogues thereof that have an aromatic group include the amino acids histidine, tryptophan, tyrosine, and phenylalanine and include the peptoid monomers ηhistidine, ηtryptophan, ηtyrosine, and ηphenylalanine For example, the hexapeptoid ηLys-ηGln-ηTyr-ηArg-ηPhe-ηTrp has a net positive charge of two (contributed by the ηlysine and ηarginine residues) and three aromatic groups (contributed by ηtyrosine, ηphenylalanine and ηtryptophan residues).

The neutral-cationic peptoids may also have a relationship between the minimum number of aromatic groups (a) and the total number of net positive charges at physiological pH (pₜ) wherein 3a is the largest number that is less than or equal to pₜ+1, except that when pₜ is 1, a may also be 1. In this embodiment, the relationship between the minimum number of aromatic groups (a) and the total number of net positive charges (pₜ) is as follows:

TABLE 3
Aromatic groups and net positive charges (3a ≤ pₜ + 1 or a = pₜ = 1)

| | | | | | | | | | | | | | | | | | | | | |
|---|---|---|---|---|---|---|---|---|---|---|---|---|---|---|---|---|---|---|---|---|
| (pₜ) | 1 | 2 | 3 | 4 | 5 | 6 | 7 | 8 | 9 | 10 | 11 | 12 | 13 | 14 | 15 | 16 | 17 | 18 | 19 | 20 |
| (a) | 1 | 1 | 1 | 1 | 2 | 2 | 2 | 3 | 3 | 3 | 4 | 4 | 4 | 5 | 5 | 5 | 6 | 6 | 6 | 7 |

In another embodiment, the neutral-cationic peptoids have a relationship between the minimum number of aromatic groups (a) and the total number of net positive charges (pₜ) wherein 2a is the largest number that is less than or equal to pₜ+1. In this embodiment, the relationship between the minimum number of aromatic monomer residues (a) and the total number of net positive charges (pₜ) is as follows:

TABLE 4
Aromatic groups and net positive charges (2a ≤ pₜ + 1 or a = pₜ = 1)

| | | | | | | | | | | | | | | | | | | | | |
|---|---|---|---|---|---|---|---|---|---|---|---|---|---|---|---|---|---|---|---|---|
| (pₜ) | 1 | 2 | 3 | 4 | 5 | 6 | 7 | 8 | 9 | 10 | 11 | 12 | 13 | 14 | 15 | 16 | 17 | 18 | 19 | 20 |
| (a) | 1 | 1 | 2 | 2 | 3 | 3 | 4 | 4 | 5 | 5 | 6 | 6 | 7 | 7 | 8 | 8 | 9 | 9 | 10 | 10 |

In another embodiment, the number of aromatic groups (a) and the total number of net positive charges (pt) are equal.

In some embodiments, carboxyl groups, especially the terminal carboxyl group of a C-terminal monomer, are amidated with, for example, ammonia to form the C-terminal amide. Alternatively, the terminal carboxyl group of the C-terminal peptoid monomer may be amidated with any primary or secondary amine. The primary or secondary amine may, for example, be an alkyl, especially a branched or unbranched C₁-C₄ alkyl, aryl or aralkyl amine. Accordingly, the monomer at the C-terminus of the peptoid may be converted to an amido, N-alkylamido, N,N-dialkylamido, N-arylamido, N,N-diarylamido, N-alkyl-N-arylamido, N-aralkylamido, N,N-diaralkylamido, N-alkyl-N-aralkylamido, or N-aryl-N-aralkylamido group, such as a N-methylamido, N-ethylamido, N,N-dimethylamido, N,N-diethyl amido, N-methyl-N-ethylamido, N-phenylamido, N-phenyl-N-ethylamido, N-benzylamido, N,N-dibenzylamido, N-methyl-N-benzylamido, N-ethyl-N-benzylamido, or N-benzyl-N-phenylamido group.

The free carboxylate groups of the ηasparagine, ηglutamine, ηaspartic acid, and ηglutamic acid residues not occurring at the C-terminus of the neutral-cationic peptoids of the present technology may also be amidated or esterified wherever they occur within the peptoid. The amidation at these internal positions may be with ammonia or any of the primary or secondary amines described herein; likewise, esterification may be with any of a primary alcohols (such as methanol, ethanol, n-propanol, n-butanol, n-pentanol, isopentanol, n-hexanol, isohexanol, and the like), a secondary alcohol (such as isopropanol, sec-butanol, sec-pentanol, cyclopentanol, sec-hexanol, cycohexanol, and the like), or tertiary alcohol (such as tert-butanol, tert-pentanol, and the like).

In one embodiment, the neutral-cationic peptoid useful in the methods of the present technology is a tripeptoid having two net positive charges and at least one aromatic monomer. In a particular embodiment, the neutral-cationic peptoid useful in the methods of the present technology is a tripeptoid having two net positive charges and two aromatic monomers.

In some embodiments, the neutral-cationic peptoid is a peptoid having:

at least one net positive charge;

a minimum of four monomer residues;

a maximum of about twenty monomer residues;

a relationship between the minimum number of net positive charges (pₘ) and the total number of monomer residues (r) wherein 3 pₘ is the largest number that is less than or equal to r+1; and a relationship between the minimum number of aromatic groups (a) and the total number of net positive charges (pₜ) wherein 2a is the largest number that is less than or equal to pₜ+1, except that when a is 1, pₜ may also be 1.

In one embodiment, 2 pₘ is the largest number that is less than or equal to r+1, and a may be equal to pₜ. The neutral-cationic peptoid may be a water-soluble peptoid having a minimum of two or a minimum of three positive charges.

In some embodiments, the C-terminal carboxyl group of the peptoid monomer at the C-terminus is amidated. In certain embodiments, the peptoid has a minimum of four monomers. The peptoid may have a total of about 6, a total of about 9, or a total of about 12 monomers.

In some embodiments, the peptoids have a ηtyrosine residue or a ηtyrosine derivative at the N-terminus (i.e., the first monomer residue position). Suitable derivatives of ηtyrosine include 2′-methyl-ηtyrosine (ηMmt); 2′, 6′-dimethyl-ηtyrosine (η2′6′-Dmt); 3′,5′-dimethyl-ηtyrosine (η3′5′Dmt); and 2′-hydroxy-6′-methyl-ηtyrosine (ηHmt).

In some embodiments, a peptoid has the formula ηTyr-ηArg-ηPhe-ηLys-NH₂. ηTyr-ηArg-FηPhe-ηLys-NH₂ has a net positive charge of three, contributed by the peptoid monomers ηtyrosine, ηarginine, and ηlysine and has two aromatic groups contributed by the peptoid monomers ηphenylalanine and ηtyrosine. The ηtyrosine of ηTyr-ηArg-FηPhe-ηLys-NH₂ can be a modified derivative of ηtyrosine such as in 2′,6′-dimethyl-ηtyrosine to produce the compound having the formula η2′,6′-Dmt-ηArg-ηPhe-ηLys-NH₂. η2′,6′-Dmt-ηArg-ηPhe-ηLys-NH₂ has a molecular weight of 640 and carries a net three positive charge at physiological pH. η2′,6′-Dmt-ηArg-ηPhe-ηLys-NH₂ is expected to readily penetrate the plasma membrane of several mammalian cell types in an energy-independent manner (Zhao et al., J. Pharmacol Exp Ther., 304:425-432, 2003).

Alternatively, in some embodiments, the neutral-cationic peptoid does not have a ηtyrosine residue or a derivative of ηtyrosine at the N-terminus (i.e., monomer position 1). The monomer at the N-terminus can be any peptoid monomer other than ηtyrosine, including peptoid monomer analogues of naturally-occurring or non-naturally-occurring amino acids other than tyrosine. In some such embodiments, the monomer at the N-terminus is ηphenylalanine or its derivative. Exemplary derivatives of ηphenylalanine include 2′-methyl-ηphenylalanine (ηMmp), 2′,6′-dimethyl-ηphenylalanine (η2′,6′-Dmp), and 2′-hydroxy-6′-methyl-ηphenylalanine (ηHmp).

An example of a neutral-cationic peptoid that does not have a ηtyrosine residue or a derivative of ηtyrosine at the N-terminus is a peptoid with the formula ηPhe-ηArg-ηPhe-ηLys-NH₂. Alternatively, the N-terminal ηphenylalanine can be a derivative of ηphenylalanine such as 2′,6′-dimethyl-ηphenylalanine (η2′6′-Dmp). In one embodiment, the monomer sequence of η2′,6′-Dmt-ηArg-ηPhe-ηLys-NH₂ is rearranged such that ηDmt is not at the N-terminus. An example of such a neutral-cationic peptoid is a peptoid having the formula of ηArg-η2′6′-Dmt-ηLys-ηPhe-NH₂.

Suitable substitution variants of the peptoids listed herein include conservative peptoid monomer substitutions. Peptoid monomers may be grouped according to their physicochemical characteristics, including the following non-exclusive listing:
(a) Non-polar peptoid monomers: ηAla(ηA), ηSer(ηS), ηThr(ηT), Gly(G), ηCys (ηC);
(b) Acidic peptoid monomers: ηAsn(ηN), ηAsp(D), ηGlu(ηE), ηGln(ηQ);
(c) Basic peptoid monomers: ηHis(ηH), ηArg(ηR), ηLys(ηK);
(d) Hydrophobic peptoid monomers: ηMet(ηM), ηLeu(ηL), Ile(ηI), ηVal(ηV); and
(e) Aromatic peptoid monomers: ηPhe(ηF), ηTyr(ηY), ηTrp(ηW).

Substitutions of a peptoid monomer in a pepoid by another peptoid monomer in the same group are referred to as a conservative substitution and may preserve the physicochemical characteristics of the original peptoid. In contrast, substitutions of an peptoid monomer in a peptoid by another peptoid monomer in a different group are generally more likely to alter the characteristics of the original peptoid.

Any amino acids in the peptoids disclosed herein may be in either the L- or the D-configuration.

### III. Uses of Compositions of the Present Technology

In some aspects, the methods disclosed herein provide therapies for the treatment of medical disease or conditions and/or side effects associated with existing therapeutics against medical diseases or conditions comprising administering an effective amount of a neutral-cationic peptoid or pharmaceutically acceptable salt thereof, such as acetate, tartrate or trifluoroacetate.

In another aspect, the present technology provides methods for treating, ameliorating or preventing a medical disease or condition in a subject in need thereof, comprising administering a therapeutically effective amount of a composition comprising a neutral-cationic peptoid of the present technology to the subject thereby treating, amelioration or preventing the medical disease or condition.

Ischemia in a tissue or organ of a mammal is a multifaceted pathological condition which is caused by oxygen deprivation (hypoxia) and/or glucose (e.g., substrate) deprivation. Oxygen and/or glucose deprivation in cells of a tissue or organ leads to a reduction or total loss of energy generating capacity and consequent loss of function of active ion transport across the cell membranes. Oxygen and/or glucose deprivation also leads to pathological changes in other cell membranes, including permeability transition in the mitochondrial membranes. In addition other molecules, such as apoptotic proteins normally compartmentalized within the mitochondria, may leak out into the cytoplasm and cause apoptotic cell death. Profound ischemia can lead to necrotic cell death.

Ischemia or hypoxia in a particular tissue or organ may be caused by a loss or severe reduction in blood supply to the tissue or organ. The loss or severe reduction in blood supply may, for example, be due to thromboembolic stroke, coronary atherosclerosis, or peripheral vascular disease. The tissue affected by ischemia or hypoxia is typically muscle, such as cardiac, skeletal, or smooth muscle.

The organ affected by ischemia or hypoxia may be any organ that is subject to ischemia or hypoxia. Examples of organs affected by ischemia or hypoxia include brain, heart, kidney, and prostate. For instance, cardiac muscle ischemia or hypoxia is commonly caused by atherosclerotic or thrombotic blockages which lead to the reduction or loss of oxygen delivery to the cardiac tissues by the cardiac arterial and capillary blood supply. Such cardiac ischemia or hypoxia may cause pain and necrosis of the affected cardiac muscle, and ultimately may lead to cardiac failure.

Ischemia or hypoxia in skeletal muscle or smooth muscle may arise from similar causes. For example, ischemia or hypoxia in intestinal smooth muscle or skeletal muscle of the limbs may also be caused by atherosclerotic or thrombotic blockages.

Reperfusion is the restoration of blood flow to any organ or tissue in which the flow of blood is decreased or blocked. For example, blood flow can be restored to any organ or tissue affected by ischemia or hypoxia. The restoration of blood flow (reperfusion) can occur by any method known to those in the art. For instance, reperfusion of ischemic cardiac tissues may arise from angioplasty, coronary artery bypass graft, or the use of thrombolytic drugs.

In some embodiments, neutral-cationic peptoids (or tautomers, regioisomers, stereoisomers, derivatives, analogues, or pharmaceutically acceptable salts thereof) may also be administered to a mammal taking a drug to treat a condition or disease.

The present disclosure provides a method for the treatment or prevention of cardiac ischemia-reperfusion injury. Also provided is a method of treating a myocardial infarction in a subject to prevent injury to the heart upon reperfusion. In one aspect, the present technology relates to a method of coronary revascularization comprising administering to a mammalian subject a therapeutically effective amount of a neutral-cationic peptoid (or tautomers, regioisomers, stereoisomers, derivatives, analogues, or pharmaceutically acceptable salts thereof), and subsequently performing a coronary artery bypass graft (CABG) procedure on the subject, where the method treats or prevents cardiac ischemia-reperfusion injury.

In some embodiments, neutral-cationic peptoids (or tautomers, regioisomers, stereoisomers, derivatives, analogues, or pharmaceutically acceptable salts thereof) are useful for both prophylactic and therapeutic methods of treating a subject at risk of (or susceptible to) cardiac ischemia-reperfusion injury. Accordingly, the present methods provide for the prevention and/or treatment of cardiac ischemia-reperfusion injury in a subject by administering an effective amount of neutral-cationic peptoids (or tautomers, regioisomers, stereoisomers, derivatives, analogues, or pharmaceutically acceptable salts thereof) to a subject in need thereof or a subject having a coronary artery bypass graft (CABG) procedure.

In therapeutic applications, compositions or medicaments comprising neutral-cationic peptoids (or tautomers, regioisomers, stereoisomers, derivatives, analogues, or pharmaceutically acceptable salts thereof) are administered to a subject suspected of, or already suffering from such a disease in an amount sufficient to cure, or partially arrest, the symptoms of the disease, including its complications and intermediate pathological phenotypes in development of the disease. As such, the technology provides methods of treating an individual afflicted with cardiac ischemia-reperfusion injury by administering an effective amount of neutral-cationic peptoids (or tautomers, regioisomers, stereoisomers, derivatives, analogues, or pharmaceutically acceptable salts thereof), and performing a CABG procedure.

In some embodiments, neutral-cationic peptoids (or tautomers, regioisomers, stereoisomers, derivatives, analogues, or pharmaceutically acceptable salts thereof) are useful for both prophylactic and therapeutic methods of treating a subject having or at risk of (susceptible to) heart failure. Accordingly, the present methods provide for the prevention and/or treatment of heart failure in a subject by administering an effective amount of neutral-cationic peptoids (or tautomers, regioisomers, stereoisomers, derivatives, analogues, or pharmaceutically acceptable salts thereof) to a subject in need thereof.

One aspect of the technology includes methods of treating heart failure in a subject for therapeutic purposes. In therapeutic applications, compositions or medicaments comprising neutral-cationic peptoids (or tautomers, regioisomers, stereoisomers, derivatives, analogues, or pharmaceutically acceptable salts thereof), are administered to a subject suspected of, or already suffering from such a disease in an amount sufficient to cure, or partially arrest, the symptoms of the disease, including its complications and intermediate pathological phenotypes in development of the disease. As such, the present technology provides methods of treating an individual afflicted with heart failure.

Subjects suffering from heart failure can be identified by any or a combination of diagnostic or prognostic assays known in the art. For example, typical symptoms of heart failure include shortness of breath (dyspnea), fatigue, weakness, difficulty breathing when lying flat, and swelling of the legs, ankles, or abdomen (edema). The subject may also be suffering from other disorders including coronary artery disease, systemic hypertension, cardiomyopathy or myocarditis, congenital heart disease, abnormal heart valves or valvular heart disease, severe lung disease, diabetes, severe anemia hyperthyroidism, arrhythmia or dysrhythmia and myocardial infarction. The primary signs of congestive heart failure are: cardiomegaly (enlarged heart), tachypnea (rapid breathing; occurs in the case of left side failure) and hepatomegaly (enlarged liver; occurs in the case of right side failure). Acute myocardial infarction (“AMI”) due to obstruction of a coronary artery is a common initiating event that can lead ultimately to heart failure. However, a subject that has AMI does not necessarily develop heart failure. Likewise, subjects that suffer from heart failure do not necessarily suffer from an AMI.

In one aspect, the present technology provides a method for preventing heart failure in a subject by administering to the subject neutral-cationic peptoids (or tautomers, regioisomers, stereoisomers, derivatives, analogues, or pharmaceutically acceptable salts thereof), that prevent the initiation or progression of the infarction. Subjects at risk for heart failure can be identified by, e.g., any or a combination of diagnostic or prognostic assays as described herein. In prophylactic applications, pharmaceutical compositions or medicaments of neutral-cationic peptoids (or tautomers, regioisomers, stereoisomers, derivatives, analogues, or pharmaceutically acceptable salts thereof), are administered to a subject susceptible to, or otherwise at risk of a disease or condition in an amount sufficient to eliminate or reduce the risk, or delay the onset of the disease, including biochemical, histologic and/or behavioral symptoms of the disease, its complications and intermediate pathological phenotypes presenting during development of the disease. Administration of prophylactic neutral-cationic peptoids (or tautomers, regioisomers, stereoisomers, derivatives, analogues, or pharmaceutically acceptable salts thereof), may occur prior to the manifestation of symptoms characteristic of the aberrancy, such that a disease or disorder is prevented or, alternatively, delayed in its progression.

### Determination of the Biological Effect of a Neutral-Cationic Peptoid of the Present Technology

In various embodiments, suitable in vitro or in vivo assays are performed to determine the effect of a specific composition of the present technology and whether its administration is indicated for treatment. In various embodiments, in vitro assays can be performed with representative animal models, to determine if a given neutral-cationic peptoid (or tautomers, regioisomers, stereoisomers, derivatives, analogues, or pharmaceutically acceptable salts thereof) exerts the desired effect in treating a disease or condition. Compounds for use in therapy can be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

### IV. Synthesis of Compositions of the Present Technology

The compounds useful in the methods of the present disclosure (e.g., neutral-cationic peptoid, or tautomers, regioisomers, stereoisomers, derivatives, analogues, or pharmaceutically acceptable salts thereof) may be synthesized by any method known in the art.

The neutral-cationic peptoids disclosed herein (such as η2′,6′-Dmt-ηArg-ηPhe-ηLys-NH₂, ηPhe-ηArg-ηPhe-ηLys-NH₂, or ηArg-η2′,6′-Dmt-ηLys-ηPhe-NH₂) may be synthesized by any method known in the art, such as chemical synthesis methods employed for proteins. Exemplary, non-limiting methods for chemically synthesizing proteins include those described by Stuart and Young in “Solid Phase Peptide Synthesis,” Second Edition, Pierce Chemical Company (1984), and in “Solid Phase Peptide Synthesis,” Methods Enzymol. 289, Academic Press, Inc, New York (1997). Exemplary, non-limiting methods for chemically synthesizing peptoids include those described by Seo, J., Lee, B.-C., and Zuckerman in Peptoids: Synthesis, Characterization, and Nanostructures in “Comprehensive Biomaterials,” Volume 2, Ducheyne, K. E., et al. (Eds.), pp. 53-76, Elsevier (2011) and references cited therein as well as by Tran, H., Gael, S. L., Connolly, M. D., and Zuckerman, R. N. in “Solid-phase Submonomer Synthesis of Peptoid Polymers and their Self-Assembly into Highly-Ordered Nanosheets,” J. Vis. Exp., 2011, Vol. 57, e3373 and references cited therein.

### V. Modes of Administration

Any method known to those in the art for contacting a cell, organ or tissue with compositions such as a neutral-cationic peptoid such as η2′,6′-Dmt-ηArg-ηPhe-ηLys-NH₂, ηPhe-ηArg-ηPhe-ηLys-NH₂, or ηArg-η2′,6′-Dmt-ηLys-ηPhe-NH₂, or pharmaceutically acceptable salt thereof, may be employed. Suitable methods include in vitro, ex vivo, or in vivo methods.

In vitro methods typically include cultured samples. For example, a cell may be placed in a reservoir (e.g., tissue culture plate), and incubated with a compound under appropriate conditions suitable for obtaining the desired result. Suitable incubation conditions may be readily determined by those skilled in the art.

Ex vivo methods typically include cells, organs or tissues removed from a mammal, such as a human. The cells, organs or tissues may, for example, be incubated with the compound under appropriate conditions. The contacted cells, organs or tissues are typically returned to the donor, placed in a recipient, or stored for future use. Thus, the compound is generally in a pharmaceutically acceptable carrier.

In vivo methods typically include the administration of a neutral-cationic peptoid to a mammal such as a human. When used in vivo for therapy, a neutral-cationic peptoid of the present technology is administered to a mammal in an amount effective in obtaining the desired result or treating the mammal. The effective amount is determined during pre-clinical trials and clinical trials by methods familiar to physicians and clinicians. The dose and dosage regimen will depend upon the degree of the disease or condition in the subject, the characteristics of the particular neutral-cationic peptoid of the present technology used, e.g., its therapeutic index, the subject, and the subject's history.

An effective amount of a neutral-cationic peptoid of the present technology useful in the present methods, such as in a pharmaceutical composition or medicament, may be administered to a mammal in need thereof by any of a number of well-known methods for administering pharmaceutical compositions or medicaments. The neutral-cationic peptoid of the present technology may be administered systemically or locally.

The neutral-cationic peptoid of the present technology may be formulated as a pharmaceutically acceptable salt. The term “pharmaceutically acceptable salt” means a salt prepared from a base or an acid which is acceptable for administration to a patient, such as a mammal (e.g., salts having acceptable mammalian safety for a given dosage regimen). However, it is understood that the salts are not required to be pharmaceutically acceptable salts, such as salts of intermediate compounds that are not intended for administration to a patient. Pharmaceutically acceptable salts may be derived from pharmaceutically acceptable inorganic or organic bases and from pharmaceutically acceptable inorganic or organic acids. In addition, when a neutral-cationic peptoid of the present technology contains both a basic moiety, such as an amine, pyridine or imidazole, and an acidic moiety such as a carboxylic acid or tetrazole, zwitterions may be formed and are included within the term “salt” as used herein. Salts derived from pharmaceutically acceptable inorganic bases include ammonium, calcium, copper, ferric, ferrous, lithium, magnesium, manganic, manganous, potassium, sodium, and zinc salts, and the like. Salts derived from pharmaceutically acceptable organic bases include salts of primary, secondary and tertiary amines, including substituted amines, cyclic amines, naturally-occurring amines and the like, such as arginine, betaine, caffeine, choline, N,N′dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine, trimethylamine, tripropylamine, tromethamine, and the like. Salts derived from pharmaceutically acceptable inorganic acids include salts of boric, carbonic, hydrohalic (hydrobromic, hydrochloric, hydrofluoric or hydroiodic), nitric, phosphoric, sulfamic, and sulfuric acids. Salts derived from pharmaceutically acceptable organic acids include salts of aliphatic hydroxyl acids (e.g., citric, gluconic, glycolic, lactic, lactobionic, malic, and tartaric acids), aliphatic monocarboxylic acids (e.g., acetic, butyric, formic, propionic, and trifluoroacetic acids), amino acids (e.g., aspartic and glutamic acids), aromatic carboxylic acids (e.g., benzoic, p-chlorobenzoic, diphenylacetic, gentisic, hippuric, and triphenylacetic acids), aromatic hydroxyl acids (e.g., o-hydroxybenzoic, p-hydroxybenzoic, 1-hydroxynaphthalene-2-carboxylic and 3-hydroxynaphthalene-2-carboxylic acids), ascorbic, dicarboxylic acids (e.g., fumaric, maleic, oxalic and succinic acids), glucoronic, mandelic, mucic, nicotinic, orotic, pamoic, pantothenic, sulfonic acids (e.g., benzenesulfonic, camphosulfonic, edisylic, ethanesulfonic, isethionic, methanesulfonic, naphthalenesulfonic, naphthalene-1,5-disulfonic, naphthalene-2,6-disulfonic and p-toluenesulfonic acids), xinafoic acid, acetate, tartrate, trifluoroacetate, and the like.

The neutral-cationic peptoid of the present technology described herein may be incorporated into pharmaceutical compositions for administration to a subject for the treatment or prevention of a disorder described herein. Such compositions typically include the neutral-cationic peptoid and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds may also be incorporated into the compositions.

Pharmaceutical compositions are typically formulated to be compatible with the intended route of administration. Routes of administration include, for example, parenteral (e.g., intravenous, intradermal, intraperitoneal or subcutaneous), oral, respiratory (e.g., inhalation), transdermal (topical), and transmucosal administration. Solutions or suspensions used for parenteral, intradermal, or subcutaneous application may include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity, such as sodium chloride or dextrose. The pH may be adjusted with acids or bases, such as hydrochloric acid or sodium hydroxide. The preparation may be enclosed in ampoules, disposable syringes or multiple-dose vials made of glass or plastic. For convenience of the patient or treating physician, the dosing formulation may be provided in a kit containing all necessary equipment (e.g., vials of drug, vials of diluent, syringes and needles) for a course of treatment (e.g., 7 days of treatment).

Pharmaceutical compositions suitable for injectable use may include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J., USA) or phosphate buffered saline (PBS). In all cases, a composition for parenteral administration must be sterile and should be formulated for ease of syringeability. The composition should be stable under the conditions of manufacture and storage, and must be shielded from contamination by microorganisms such as bacteria and fungi.

In one embodiment, the neutral-cationic peptoid of the present technology is administered intravenously. For example, a neutral-cationic peptoid of the present technology may be administered via rapid intravenous bolus injection. In some embodiments, the neutral-cationic peptoid of the present technology is administered as a constant-rate intravenous infusion.

The neutral-cationic peptoid of the present technology may also be administered orally, topically, intranasally, intramuscularly, subcutaneously, or transdermally. In one embodiment, transdermal administration is by iontophoresis, in which the charged composition is delivered across the skin by an electric current.

Other routes of administration include intracerebroventricularly or intrathecally. Intracerebroventricularly refers to administration into the ventricular system of the brain. Intrathecally refers to administration into the space under the arachnoid membrane of the spinal cord. Thus, in some embodiments, intracerebroventricular or intrathecal administration is used for those diseases and conditions which affect the organs or tissues of the central nervous system.

The neutral-cationic peptoid of the present technology may also be administered to mammals by sustained release, as is known in the art. Sustained release administration is a method of drug delivery to achieve a certain level of the drug over a particular period of time. The level is typically measured by serum or plasma concentration. A description of methods for delivering a compound by controlled release may be found in international PCT Application No. WO 02/083106, which is incorporated herein by reference in its entirety.

Any formulation known in the art of pharmacy is suitable for administration of the neutral-cationic peptoid of the present technology. For oral administration, liquid or solid formulations may be used. Examples of formulations include tablets, gelatin capsules, pills, troches, elixirs, suspensions, syrups, wafers, chewing gum and the like. The neutral-cationic peptoids of the present technology may be mixed with a suitable pharmaceutical carrier (vehicle) or excipient as understood by practitioners in the art. Examples of carriers and excipients include starch, milk, sugar, certain types of clay, gelatin, lactic acid, stearic acid or salts thereof, including magnesium or calcium stearate, talc, vegetable fats or oils, gums and glycols.

For systemic, intracerebroventricular, intrathecal, topical, intranasal, subcutaneous, or transdermal administration, formulations of the neutral-cationic peptoids of the present technology may utilize conventional diluents, carriers, or excipients etc., such as those known in the art to deliver the neutral-cationic peptoids of the present technology. For example, the formulations may comprise one or more of the following: a stabilizer, a surfactant, such as a nonionic surfactant, and optionally a salt and/or a buffering agent. The neutral-cationic peptoid of the present technology may be delivered in the form of an aqueous solution, or in a lyophilized form.

The stabilizer may comprise, for example, an amino acid, such as for instance, glycine; an oligosaccharide, such as, sucrose, tetralose, lactose; or a dextran. Alternatively, the stabilizer may comprise a sugar alcohol, such as, mannitol. In some embodiments, the stabilizer or combination of stabilizers constitutes from about 0.1% to about 10% weight for weight of the formulated composition.

In some embodiments, the surfactant is a nonionic surfactant, such as a polysorbate. Examples of suitable surfactants include Tween 20, Tween 80; a polyethylene glycol or a polyoxyethylene polyoxypropylene glycol, such as Pluronic F-68 at from about 0.001% (w/v) to about 10% (w/v).

The salt or buffering agent may be any salt or buffering agent, such as for example, sodium chloride, or sodium/potassium phosphate, respectively. In some embodiments, the buffering agent maintains the pH of the pharmaceutical composition in the range of about 5.5 to about 7.5. The salt and/or buffering agent is also useful to maintain the osmolality at a level suitable for administration to a human or an animal. In some embodiments, the salt or buffering agent is present at a roughly isotonic concentration of about 150 mM to about 300 mM.

Formulations of neutral-cationic peptoids of the present technology may additionally contain one or more conventional additives. Examples of such additives include a solubilizer such as, for example, glycerol; an antioxidant such as for example, benzalkonium chloride (a mixture of quaternary ammonium compounds, known as “quats”), benzyl alcohol, chloretone or chlorobutanol; an anesthetic agent such as for example a morphine derivative; and an isotonic agent etc., such as described herein. As a further precaution against oxidation or other spoilage, the pharmaceutical compositions may be stored under nitrogen gas in vials sealed with impermeable stoppers.

The mammal treated in accordance with the present technology may be any mammal, including, for example, farm animals, such as sheep, pigs, cows, and horses; pet animals, such as dogs and cats; and laboratory animals, such as rats, mice and rabbits. In one embodiment, the mammal is a human.

The neutral-cationic peptoid of the present technology may be administered systemically or locally. In one embodiment, the neutral-cationic peptoid of the present technology are administered intravenously. For example, neutral-cationic peptoid of the present technology may be administered via rapid intravenous bolus injection. In one embodiment, the neutral-cationic peptoid of the present technology is administered as a constant-rate intravenous infusion.

The neutral-cationic peptoid of the present technology may be injected directly into a coronary artery during, for example, angioplasty or coronary bypass surgery, or applied onto coronary stents.

The neutral-cationic peptoid of the present technology may include a carrier, which may be a solvent or dispersion medium containing, for example, water, ethanol, polyol (for example, glycerol, propylene glycol, and liquid polyethylene glycol, and the like), or suitable mixtures thereof. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thiomerasol, and the like. Glutathione and other antioxidants may be included in the composition to prevent oxidation. In many cases, it is desirable to include isotonic agents, for example, sugars, polyalcohols such as mannitol, sorbitol, or sodium chloride. Prolonged absorption of the injectable compositions may be brought about by including in the composition an agent which delays absorption, for example, aluminum monostearate or gelatin.

Sterile injectable solutions may be prepared by incorporating the neutral-cationic peptoid of the present technology in the required amount in an appropriate solvent with one or a combination of ingredients enumerated above, as required, followed by filtered sterilization. Generally, dispersions are prepared by incorporating the neutral-cationic peptoid of the present technology into a sterile vehicle, which contains a basic dispersion medium and the required other ingredients from those enumerated above. In the case of sterile powders for the preparation of sterile injectable solutions, typical methods of preparation include vacuum drying and freeze drying, which may yield a powder of the neutral-cationic peptoid of the present technology plus any additional desired ingredient from a previously sterile-filtered solution thereof.

Oral compositions generally include an inert diluent or an edible carrier. For the purpose of oral therapeutic administration, the neutral-cationic peptoid of the present technology may be incorporated with excipients and used in the form of tablets, troches, or capsules, e.g., gelatin capsules. Oral compositions may also be prepared using a fluid carrier for use as a mouthwash. Pharmaceutically compatible binding agents, and/or adjuvant materials may be included as part of the composition. The tablets, pills, capsules, troches and the like may contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the neutral-cationic peptoid of the present technology can be delivered in the form of an aerosol spray from a pressurized container or dispenser which contains a suitable propellant, e.g., a gas such as carbon dioxide, or a nebulizer. Such methods include those described in U.S. Pat. No. 6,468,798.

Systemic administration of a neutral-cationic peptoid of the present technology as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the neutral-cationic peptoids of the present technology are formulated into ointments, salves, gels, or creams as generally known in the art. In one embodiment, transdermal administration may be performed by iontophoresis.

A neutral-cationic peptoid of the present technology can be formulated in a carrier system. The carrier can be a colloidal system. The colloidal system can be a liposome, a phospholipid bilayer vehicle. In one embodiment, the therapeutic neutral-cationic peptoid of the present technology is encapsulated in a liposome while maintaining peptoid integrity. As one skilled in the art will appreciate, there are a variety of methods to prepare liposomes. (See Lichtenberg, et al., Methods Biochem. Anal. 33:337-462 (1988); Anselem, et al., Liposome Technology, CRC Press (1993)). Liposomal formulations can delay clearance and increase cellular uptake (See Reddy, Ann. Pharmacother. 34 (78):915-923 (2000)). A neutral-cationic peptoid can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic neutral-cationic peptoid of the present technology can be embedded in the polymer matrix, while maintaining peptoid integrity. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother. 34:915-923 (2000). A polymer formulation for human growth hormone (hGH) has been used in clinical trials. (See Kozarich and Rich, Chemical Biology 2:548-552 (1998).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the neutral-cationic peptoids of the present technology are prepared with carriers that will protect the neutral-cationic peptoids of the present technology against potential rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylacetic acid. Such formulations can be prepared using known techniques. The materials can also be obtained commercially, e.g., from Alza Corporation (Mountain View, Calif., USA) and Nova Pharmaceuticals, Inc. (Sydney, AU). Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The neutral-cationic peptoid of the present technology can also be formulated to enhance intracellular delivery. For example, liposomal delivery systems are known in the art. See, e.g., Chonn and Cullis, Curr. Opin. Biotech. 6:698-708 (1995); Weiner, Immunometh. 4(3):201-9 (1994); Gregoriadis, Trends Biotechnol. 13(12):527-37 (1995). Mizguchi, et al., Cancer Lett. 100:63-69 (1996), describes the use of fusogenic liposomes to deliver a protein to cells both in vivo and in vitro.

Dosage, toxicity and therapeutic efficacy of the neutral-cationic peptoid of the present technology can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. In some embodiments, the neutral-cationic peptoids of the present technology exhibit high therapeutic indices. While neutral-cationic peptoids that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any neutral-cationic peptoid of the present technology used in the methods described herein, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Typically, an effective amount of the neutral-cationic peptoid of the present technology, sufficient for achieving a therapeutic or prophylactic effect, range from about 0.000001 mg per kilogram body weight per day to about 10,000 mg per kilogram body weight per day. In some embodiments, the dosage ranges will be from about 0.0001 mg per kilogram body weight per day to about 100 mg per kilogram body weight per day. For example dosages can be 1 mg/kg body weight or 10 mg/kg body weight every day, every two days or every three days or within the range of 1-10 mg/kg every week, every two weeks or every three weeks. In one embodiment, a single dosage of neutral-cationic peptoid of the present technology ranges from 0.1-10,000 micrograms per kg body weight. In one embodiment, neutral-cationic peptoid concentration in a carrier ranges from 0.2 to 2000 micrograms per delivered milliliter. An exemplary treatment regimen entails administration once per day or once a week. Intervals can also be irregular as indicated by measuring blood levels of glucose or insulin in the subject and adjusting dosage or administration accordingly. In some methods, dosage is adjusted to achieve a desired fasting glucose or fasting insulin concentration. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regimen.

In some embodiments, a therapeutically effective amount of neutral-cationic peptoid of the present technology is defined as a concentration of the neutral-cationic peptoid of the present technology at the target tissue of 10⁻¹¹ to 10⁻⁶ molar, e.g., approximately 10⁻⁷ molar. This concentration may be delivered by systemic doses of 0.01 to 100 mg/kg or equivalent dose by body surface area. The schedule of doses is optimized to maintain the therapeutic concentration at the target tissue, such as by single daily or weekly administration, but also including continuous administration (e.g., parenteral infusion or transdermal application).

The skilled artisan will appreciate that certain factors may influence the dosage and timing required to effectively treat a subject, including but not limited to, the severity of the disease or disorder, previous treatments, the general health and/or age of the subject, and the presence of other diseases. Moreover, treatment of a subject with a therapeutically effective amount of the therapeutic compositions described herein can include a single treatment or a series of treatments.

### VI. Formulations

In some aspects, the present disclosure provide pharmaceutical formulations for the delivery of neutral-cationic peptoids of the present technology.

In one aspect, the present technology relates to a finished pharmaceutical product adapted for oral delivery of neutral-cationic peptoid compositions, the product comprising: (a) a therapeutically effective amount of the neutral-cationic peptoid; (b) at least one pharmaceutically acceptable pH-lowering agent; and (c) at least one absorption enhancer effective to promote bioavailability of the neutral-cationic peptoid, wherein the pH-lowering agent is present in the finished pharmaceutical product in a quantity which, if the product were added to 10 milliliters of 0.1M aqueous sodium bicarbonate solution, would be sufficient to lower the pH of the solution to no higher than 5.5, and wherein an outer surface of the product is substantially free of an acid-resistant protective vehicle.

In some embodiments, the pH-lowering agent is present in a quantity which, if the product were added to 10 milliliters of 0.1M sodium bicarbonate solution, would be sufficient to lower the pH of the solution to no higher than 3.5. In some embodiments, the absorption enhancer is an absorbable or biodegradable surface active agent. In some embodiments, the surface active agent is selected from the group consisting of acylcarnitines, phospholipids, bile acids and sucrose esters. In some embodiments, the absorption enhancer is a surface active agent selected from the group consisting of: (a) an anionic agent that is a cholesterol derivative, (b) a mixture of a negative charge neutralizer and an anionic surface active agent, (c) non-ionic surface active agents, and (d) cationic surface active agents.

In some embodiments, the finished pharmaceutical product further comprises an amount of an additional peptide that is not a physiologically active peptide effective to enhance bioavailability of the neutral-cationic peptoids of the present technology. In some embodiments, the finished pharmaceutical product comprises at least one pH-lowering agent with a solubility in water of at least 30 grams per 100 milliliters of water at room temperature. In some embodiments, the finished pharmaceutical product comprises granules containing a pharmaceutical binder and, uniformly dispersed in the binder, the pH-lowering agent, the absorption enhancer and the neutral-cationic peptoids of the present technology.

In some embodiments, the finished pharmaceutical product comprises a lamination having a first layer comprising at least one pharmaceutically acceptable pH-lowering agent and a second layer comprising the therapeutically effective amount of the neutral-cationic peptoid; the product further comprising the at least one absorption enhancer effective to promote bioavailability of the neutral-cationic peptoid, wherein the first and second layers are united with each other, but the at least one pH-lowering agent and the neutral-cationic peptoid are substantially separated within the lamination such that less than about 0.1% of the neutral-cationic peptoid contacts the pH-lowering agent to prevent substantial mixing between the first layer material and the second layer material and thus to avoid interaction in the lamination between the pH-lowering agent and the neutral-cationic peptoid.

In some embodiments, the finished pharmaceutical product comprises a pH-lowering agent selected from the group consisting of citric acid, tartaric acid and an acid salt of an amino acid. In some embodiments, the pH-lowering agent is selected from the group consisting of dicarboxylic acids and tricarboxylic acids. In some embodiments, the pH-lowering agent is present in an amount not less than 300 milligrams.

### VII. Examples

The present technology is further illustrated by the following examples, which should not be construed as limiting in any way. For each of the examples below, any neutral-cationic peptoid described herein could be used. By way of example, but not by limitation, the neutral-cationic peptoid used in the examples below could be η2′6′-Dmt-ηArg-ηPhe-ηLys-NH₂, ηPhe-ηArg-ηPhe-ηLys-NH₂, or ηArg-η2′6′-Dmt-ηLys-ηPhe-NH₂ or any one or more of the peptoids shown in Section II.

### Example 1: Effects of Neutral-Cationic Peptoids in Protecting Against Cardiac Ischemia-Reperfusion Injury in a Guinea Pig Model

The effects of neutral-cationic peptoids in protecting against cardiac ischemia-reperfusion injury in a guinea pig model and the myocardial protective effect of neutral-cationic peptoids may further be evidenced by this Example.

Experimental Methods

Procedures for the use of guinea pigs are in accordance with the guidelines established by the American Physiological Society. Adult male guinea pigs (200-300 g) will be anesthetized with a ketamine/xylazine cocktail (85/15 mg mL, respectively; ip delivery). Upon the absence of reflexes to ensure a deep plane of anesthesia, hearts will be excised via midline thoracotomy and immersed in ice-cold saline. Hearts will be cannulated by the aorta and perfused with a modified Krebs-Henseleit buffer containing (in mM): 118 NaCl, 24 NaHCO₃, 4.75 KCl, 1.2 KH₂PO₄, 1.2 MgSO₄, 2.0 CaCl₂, and 10 glucose (gassed with 95/5% O2/CO₂). Hearts will be placed in a buffer-filled perfusion chamber and maintained at 37° C. for the duration of the experiments.

Following the initiation of perfusion, hearts will be instrumented for the simultaneous observation of mechanical and electrical function. A buffer-filled latex balloon will be inserted into the left ventricle (via the mitral valve) for the measurement of left ventricular developed pressure, with balloon volume adjusted to establish an end-diastolic pressure of 5-8 mmHg. Three electrodes will be placed into the buffer-filled perfusion chamber for the measurement of volume-conducted ECG. A pre-established protocol of electrode placement will be utilized to obtain a signal analogous to Lead II of a typical 12-lead ECG. All physiological parameters will be continuously monitored and stored on a personal computer using commercially available software (Chart, AD Instruments).

After a 10-minute equilibration period, hearts will be divided into the following different treatment groups: 1. Control followed by I/R; 2. Administration of 1 nM ηArg-η2′6′-Dmt-ηLys-ηPhe-NH₂ in the perfusate both before and after index ischemia; 3. Post-ischemic administration of 1 nM ηArg-η2′6′-Dmt-ηLys-ηPhe-NH₂ using both a bolus dose (also 1 nM, administered immediately prior to reperfusion) and ηArg-η2′6′-Dmt-ηLys-ηPhe-NH₂ in the reperfusion solution; 4. Positive control using ischemic preconditioning; 3 cycles of 5 min I/R before index ischemia).

Methods: Ischemia/Reperfusion

Hearts will be exposed to global no-flow ischemia by stopping perfusion for 20 minutes. At the end of the index ischemia, static buffer from the perfusion lines will be washed out (via an accessory port proximal to the aortic cannula) and reperfusion ensued for 120 minutes. Administration of all compounds in the perfusate will be accomplished via dissolving the compound(s) in solution prior to administration. The reperfusion bolus dose will be delivered to the heart via syringe through a drug-delivery port just above the aortic cannula. At the end of the 2 h reperfusion protocol, the LV will be dissected, sliced into 5 mm-thick slices, incubated in triphenyltetrazolium chloride (TTC) for 10 minutes (37° C.), and digitally photographed for subsequent infarct size analysis. Infarct sizes are expressed as the infarcted area as a percentage of the LV (in the global ischemia model, the entire LV constitutes the zone-at-risk).

Results

Infarct Size.

Hearts will be exposed to 20 minutes of global ischemia. Hearts that are treated with either 1 nM or 0.1 nM or 0.001 nM or 0.01 nM/0.1 nM(30 min R) or ηArg-η2′6′-Dmt-ηLys-ηPhe-NH₂ are expected to exhibit lower incidence of infarction (infarction size) when compared to control group. For these ηArg-η2′6′-Dmt-ηLys-ηPhe-NH₂ treatment groups, the time of drug administration (i.e., pre-versus post-ischemic) is not expected to influence the magnitude of efficacy. Cyclosporin (0.2 μM) is expected to significantly attenuate I/R injury, but only when administered prior to ischemia. There is expected to be a strong trend for cyclosporin to reduce infarct size when administered at reperfusion.

Incidence of Arrhythmia.

The effect of ηArg-η2′6′-Dmt-ηLys-ηPhe-NH₂ against protection from arrhythmia will be studied. The isolated guinea pig heart exposed to global ischemia is expected to exhibit reproducible ventricular arrhythmia at the onset of reperfusion. Almost all hearts in the study are expected to exhibit some degree of ventricular tachycardia and/or fibrillation (VT/VF) during the protocol. Hearts that receive 0.01 nM/0.1 nM(30 min R) ηArg-η2′6′-Dmt-ηLys-ηPhe-NH₂ are expected to show protection against the incidence of VT/VF. A higher concentration of ηArg-η2′6′-Dmt-ηLys-ηPhe-NH₂ (1 nM) is not expected to be effective in reducing the amount of time that hearts spent in VT/VF. Cyclosporin (0.2 μM) showed modest efficacy in reducing the time hearts spent in ventricular arrhythmia, but this slight decrease did not reach statistical significance.

Coronary Flow Rates.

Coronary Flow rates will be monitored continuously and expressed as mL/min*g of whole heart wet weight. Treatment groups will be control, 1 nM ηArg-η2′6′-Dmt-ηLys-ηPhe-NH₂ whole time; 1 nM ηArg-η2′6′-Dmt-ηLys-ηPhe-NH₂ at reperfusion; 0.1 nM ηArg-η2′6′-Dmt-ηLys-ηPhe-NH₂ at reperfusion; 0.01 nM ηArg-η2′6′-Dmt-ηLys-ηPhe-NH₂ to 0.1 nM whole time; 0.01 nM ηArg-η2′6′-Dmt-ηLys-ηPhe-NH₂ to 0.1 nM at reperfusion; 0.2 uM cyclosporin-A whole time; and 0.2 uM cyclosporin-A at reperfusion. There will be no differences in coronary flow rates. In groups receiving 0.01 nM ηArg-η2′6′-Dmt-ηLys-ηPhe-NH₂, baseline flow rates are expected to be slightly lower than other groups.

Left Ventricular Developed Pressure.

The effects of ηArg-η2′6′-Dmt-ηLys-ηPhe-NH₂ on baseline myocardial mechanical function will be examined. It is expected that there will be no discernable effects of ηArg-η2′6′-Dmt-ηLys-ηPhe-NH₂ (at any concentration (0.01 nM, 0.1 nM) or cyclosporin-A (0.2 μM) on baseline left ventricular developed pressure, heart rate, or rates of contraction/relaxation.

The results are expected to show that ηArg-η2′6′-Dmt-ηLys-ηPhe-NH₂ is useful to prevent or treat ischemia/reperfusion injury of the heart in a subject in need thereof.

### Example 2. Effect of Neutral-Cationic Peptoids on Myocardial Function and Response to Reperfusion Injury in the Modified Langendorff Model

In this example, the ability of neutral-cationic peptoids to prevent ischemia/reperfusion (IR) injury will be assessed.

Modified Langendorff Model

The Langendorff rodent heart model is widely employed in studies of myocardial function and responses to injury (e.g., ischaemia). For whole-heart studies, male Sprague-Dawley rats (7-9 weeks old) will be injected with pentobarbital (35 m/kg, ip injection) and hearts excised with midline thoracotomy. The aortas will be secured around a cannula of a modified Langendorff apparatus and retrogradely perfused (perfusion pressure of 75 mm Hg) with a modified Krebs-Henseleit buffer containing (in mM): 118 NaCl, 24 NaHCO₃, 4.75 KCl, 1.2 KH₂PO₄, 1.2 MgSO₄, 2.0 CaCl₂, and 10 glucose (gassed with 95/5% O₂/CO₂). Hearts will be bathed in a buffer-filled perfusion chamber maintained at 37° C. for the duration of the experiments. Following the initiation of perfusion, hearts will be instrumented for the simultaneous observation of mechanical and electrical function. A buffer-filled latex balloon (size 5, Harvard Apparatus, Holliston, Mass., USA), calibrated at the beginning of each day using a digital manometer, will be inserted into the left ventricle (via the mitral valve) for the measurement of left ventricular developed pressure (LVDP), with balloon volume adjusted to establish a diastolic pressure of 5-8 mm Hg. Three electrodes will be placed into the buffer filled perfusion chamber for the measurement of the volume-conducted electrocardiogram (ECG). Coronary flow rates will be monitored constantly with a flow probe (Transconic Systems, Ithaca, N.Y., USA) connected in series with the perfusion line, and normalized to heart wet weight (in grams) at the end of each experiment. All physiological parameters will be continuously monitored and stored on a personal computer using commercially available software (e.g., Chart, AD Instruments, Colorado Springs, Colo., USA). Heart rate will be calculated using the LVDP trace, and maximal rates of contraction and relaxation (±dP/dt) will be calculated using the derivative of the LVDP trace.

Ischemia/Reperfusion Protocol and Peptoid Treatments

Following a 10 minute baseline period, ischemia/reperfusion will be initiated. Hearts will be exposed to global no-flow ischemia by stopping perfusion for 20 min. At the end of the index ischemia, static buffer from the perfusion lines will be washed out (via an accessory port proximal to the aortic cannula), and reperfusion will be ensued for 2 h either with Krebs buffer alone (control) or Krebs buffer containing a predetermined concentration of the neutral-cationic peptoid. At the end of reperfusion, the left ventricle will be dissected, sliced into 5 mm-thick slices, incubated in 1% triphenyltetrazolium chloride (TTC) for 10 min (37° C.) and digitally photographed for subsequent infarct size analysis. Infarct size will be expressed as a percentage of the left ventricle (% area at risk (AAR))(calculated using ImageJ software, NIH, Bethesda, Md., USA).

Results

The results are expected to show that treatment with a neutral-cationic peptoid of the present technology significantly decrease infarct size and LVDP, and/or increases the maximal rates of contraction and relaxation (±dP/dt). Thus, the results are expected to show that neutral-cationic peptoids of the present technology are useful to prevent or treat ischemia/reperfusion injury of the heart in a subject in need thereof.

### REFERENCES

1. Herlitz J, Bengtson A, Hjalmarson A, Karlson B W. Morbidity during five years after myocardial infarction and its relation to infarct size. Clin Cardiol., 1988 October, 11(10):672-7.
2. Herlitz J, Hjalmarson A, Waldenstrom J. Relationship between enzymatically estimated infarct size and short- and long-term survival after acute myocardial infarction. Acta Med Scand., 1984; 216(3):261-7.
3. Miller T D, Christian T F, Hopfenspirger M R, Hodge D O, Gersh B J, Gibbons R J. Infarct size after acute myocardial infarction measured by quantitative tomographic 99mTc sestamibi imaging predicts subsequent mortality. Circulation, 1995 Aug. 1, 92(3):334-41.
4. Bolli R. Preconditioning: a paradigm shift in the biology of myocardial ischemia. American Journal of Physiology, 2007 January, 292(1):H19-27.
5. Bolli R, Jeroudi M O, Patel B S, DuBose C M, Lai E K, Roberts R, et al. Direct evidence that oxygen-derived free radicals contribute to postischemic myocardial dysfunction in the intact dog. Proc Natl Acad Sci USA, 1989 June, 86(12):4695-9.
6. Woodward B, Zakaria M N. Effect of some free radical scavengers on reperfusion induced arrhythmias in the isolated rat heart. Journal of Molecular and Cellular Cardiology, 1985 May, 17(5):485-93.
7. Yellon D M, Dana A. The preconditioning phenomenon: A tool for the scientist or a clinical reality? Circulation Research, 2000 September, 29; 87(7):543-50.
8. Yellon D M, Downey J M. Preconditioning the myocardium: from cellular physiology to clinical cardiology. Physiological Reviews, 2003 October, 83(4):1113-51.
9. Konya L, Kekesi V, Juhasz-Nagy S, Feher J. The effect of superoxide dismutase in the myocardium during reperfusion in the dog. Free Radical Biology & Medicine, 1992 November, 13(5):527-32.
10. Chi L G, Tamura Y, Hoff P T, Macha M, Gallagher K P, Schork M A, et al. Effect of superoxide dismutase on myocardial infarct size in the canine heart after 6 hours of regional ischemia and reperfusion: a demonstration of myocardial salvage. Circulation Research, 1989 April, 64(4):665-75.
11. Kilgore K S, Friedrichs G S, Johnson C R, Schasteen C S, Riley D P, Weiss R H, et al. Protective effects of the SOD-mimetic SC-52608 against ischemia/reperfusion damage in the rabbit isolated heart. Journal of Molecular and Cellular Cardiology, 1994 August, 26(8):995-1006.
12. Bognar Z, Kalai T, Palfi A, Hanto K, Bognar B, Mark L, et al. A novel SODmimetic permeability transition inhibitor agent protects ischemic heart by inhibiting both apoptotic and necrotic cell death. Free Radical Biology & Medicine, 2006 Sep. 1, 41(5):835-48.
13. Jones S P, Hoffineyer M R, Sharp B R, Ho Y S, Lefer D J. Role of intracellular antioxidant enzymes after in vivo myocardial ischemia and reperfusion. American Journal of Physiology, 2003 January, 284(1):H277-82.
14. Jones D P. Radical-free biology of oxidative stress. Am J Physic Cell Physiol, 2008 October, 295(4):C849-68.
15. Flaherty J T, Pitt B, Gruber J W, Heuser R R, Rothbaum D A, Burwell L R, et al. Recombinant human superoxide dismutase (h-SOD) fails to improve recovery of ventricular function in patients undergoing coronary angioplasty for acute myocardial infarction. Circulation, 1994 May, 89(5):1982-91.
16. Tsujita K, Shimomura H, Kawano H, Hokamaki J, Fukuda M, Yamashita T, et al. Effects of edaravone on reperfusion injury in patients with acute myocardial infarction. The American Journal of Cardiology. 2004 Aug. 15, 94(4):481-4.
17. Szeto H H. Mitochondria-targeted cytoprotective peptides for ischemia reperfusion injury. Antioxidants & Redox Signaling, 2008 March, 10(3):601-19.
18. Cho J, Won K, Wu D, Soong Y, Liu S, Szeto H H, et al. Potent mitochondria-targeted peptides reduce myocardial infarction in rats. Coronary Artery Disease, 2007 May, 18(3):215-20.
19. Curtis M J, Walker M J. Quantification of arrhythmias using scoring systems: an examination of seven scores in an in vivo model of regional myocardial ischaemia. Cardiovascular Research, 1988 September, 22(9):656-65.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

What is claimed is:

1. A composition comprising a neutral-cationic peptoid as described in Section II.

2. The composition of claim 1, wherein the neutral-cationic peptoid is of Formulas I, II, or III.

3. A method for treating or preventing a disease or condition, comprising administering a therapeutically effective amount of a composition comprising a neutral-cationic peptoid as described in Section II.

4. The method of claim 3, wherein the disease or condition comprises ischemia, reperfusion, ischemic heart disease, vessel occlusion injury, and/or myocardial infarction.

5. The method of claim 3, wherein a subject is suffering from ischemia or has an anatomic zone of no-reflow in one or more of cardiovascular tissue, skeletal muscle tissue, cerebral tissue and renal tissue.

6. The method of claim 3, wherein the subject is diagnosed as having, suspected of having, or at risk of having, ischemia such as cerebral ischemia and myocardial ischemia or ischemia-reperfusion.

7. A method for treating or preventing no reflow following ischemia-reperfusion injury in a subject in need thereof, comprising administering to the subject an effective amount of a composition comprising a neutral-cationic peptoid as described in Section II.

8. The composition of claim 1, further comprising one or more of at least one pharmaceutically acceptable pH-lowering agent; and at least one absorption enhancer effective to promote bioavailability of the neutral-cationic peptoid, and one or more lamination layers.

9. The composition of claim 8, wherein the pH-lowering agent is selected from the group consisting of citric acid, tartaric acid and an acid salt of an amino acid.
